(19) United States
(12) Patent Application Publication
(10) Pub. No.: US 2026/0151484 A1
(43) Pub. Date: Jun. 4, 2026

(54) COMPOSITIONS AND METHODS FOR GENERATING NEO-ANTIGEN REACTIVE TUMOR INFILTRATING LYMPHOCYTES

(71) Applicant: Turnstone Biologics Corp., La Jolla, CA (US)

(72) Inventors: James Kenneth NIKOTA, Brantford (CA); Larissa A. PIKOR, Ottawa (CA); Kwame TWUMASI-BOATENG, Ottawa (CA); David F. STOJDL, Ottawa (CA); Timothy J. LANGER, Topanga, CA (US)

(73) Assignee: Turnstone Biologics Corp., La Jolla, CA (US)

(21) Appl. No.: 19/126,587

(22) PCT Filed: Nov. 3, 2023
(86) PCT No.: PCT/US2023/078751

§ 371 Date: May 1, 2025

Related U.S. Application Data
(60) Provisional application No. 63/422,914, filed on Nov. 4, 2022.
(60) Provisional application No. 63/490,000, filed on Mar. 14, 2023.
(60) Provisional application No. 63/594,405, filed on Oct. 30, 2023.

(51) Int. Cl. A61K 40/42; A61K 40/11; A61K 40/32; A61P 35/00; C12N 5/0783

(52) U.S. Cl. CPC A61K 40/428; A61K 40/11; A61K 40/32; A61P 35/00; C12N 5/0636; C12N 2502/30

(57) ABSTRACT

Various embodiments of the invention provide compositions of tumor infiltrating lymphocytes (TILs) enriched in tumor reactive cells, methods for manufacturing TILs enriched in tumor reactive cells and methods and uses of the provided enriched tumor reactive TILs for treating cancer in a human or other subject. Among the provided embodiments of TIL compositions may include those that exhibit substantial tumor reactivity activity, including degranulation and the ability to express one or more of IFN-gamma and TNF-alpha, in response to antigen presenting cells presenting neo antigenic peptides.

### CROSS-REFERENCE TO RELATED APPLICATIONS

This application claims priority from U.S. provisional application No. 63/422,914, filed Nov. 4, 2022, entitled “COMPOSITIONS AND METHODS FOR GENERATING NEO-ANTIGEN REACTIVE TUMOR INFILTRATING LYMPHOCYTES”, U.S. provisional application No. 63/490,000, filed Mar. 14, 2023, entitled “COMPOSITIONS AND METHODS FOR GENERATING NEO-ANTIGEN REACTIVE TUMOR INFILTRATING LYMPHOCYTES”, and U.S. provisional application No. 63/594,405, filed Oct. 30, 2023, entitled “COMPOSITIONS AND METHODS FOR GENERATING NEO-ANTIGEN REACTIVE TUMOR INFILTRATING LYMPHOCYTES”, the contents of which are incorporated by reference in their entirety.

### FIELD

Embodiments of the invention relate to compositions of tumor infiltrating lymphocytes (TILs) enriched in tumor reactive cells. Embodiments of the invention also relate to methods for manufacturing TILs enriched in tumor reactive cells and uses of the provided enriched tumor reactive TILs for treating cancer in a subject.

### BACKGROUND

The process for producing autologous tumor infiltrating lymphocyte (TIL) therapies involves a low number of reactive cells resulting in compositions that are not entirely suitable for therapeutic use including for the treatment of cancer. Therefore, it would be desirable to develop improvements to the TIL compositions to overcome these limitations. Provided herein are embodiments that meet such needs.

### SUMMARY OF THE INVENTION

Various embodiments of the invention provide pharmaceutical T lymphocyte infiltrating (TIL) compositions enriched in tumor reactive T cells, methods of producing TIL compositions enriched in tumor reactive T-cells and methods of using such compositions in various therapies including for the treatment of cancer and other disease.

According to an embodiment, a pharmaceutical composition comprises a multiclonal population of T cells comprising CD4+ and CD8+ T cells from a tumor, wherein the population is enriched in at least 10 different T cell receptor (TCR) clonotypes each with a frequency in the population of at least 1.0%; and wherein at least 90% of the cells in the composition are CD3+ T cells. According to an embodiment, a pharmaceutical composition comprises an oligoclonal population of T cells comprising CD4+ and CD8+ T cells from a tumor, wherein the population is enriched in at least 10 different T cell receptor (TCR) clonotypes each with a frequency in the population of at least 1.0%; and wherein at least 90% of the cells in the composition are CD3+ T cells. According to an embodiment, a pharmaceutical composition comprises a multiclonal population of T cells comprising CD4+ and CD8+ T cells from a tumor, wherein the population is enriched in at least 10 different T cell receptor (TCR) clonotypes each with a frequency in the population of at least 2.0%; and wherein at least 90% of the cells in the composition are CD3+ T cells. According to an embodiment, a pharmaceutical composition comprises an oligoclonal population of T cells comprising CD4+ and CD8+ T cells from a tumor, wherein the population is enriched in at least 10 different T cell receptor (TCR) clonotypes each with a frequency in the population of at least 2.0%; and wherein at least 90% of the cells in the composition are CD3+ T cells. Various embodiments of the invention are particularly useful for treating cancer including difficult to treat cancers such as those with mutations unique to a particular patient including instances where such mutation have rendered the cancer resistant or refractory to standard cancer treatment (e.g., chemotherapy).

According to one or more embodiments, the multiclonal population of T cells have a minimum number of different TCR clonotypes which make up a selected percentage in the population. According to one or more embodiments, the oligoclonal population of T cells have a minimum number of different TCR clonotypes which make up a selected percentage in the population. For example, in one or more embodiments, at least 11 different TCR clonotypes have a frequency in the population of at least 2.0%, or at least 12 different TCR clonotypes have a frequency in the population of at least about 2.0%. Further, in one or more embodiments, at least 11 different TCR clonotypes have a frequency in the population of at least 1.0%, or at least 12 different TCR clonotypes have a frequency in the population of at least about 1.0%. According to some embodiments, 8 to 15 different T cell receptor (TCR) clonotypes make up at least about 50% of the TCR frequency in the population.

According to some embodiments, a pharmaceutical composition enriched in tumor reactive T cells comprises a multiclonal population of tumor infiltrating lymphocytes comprising CD4+ and CD8+ T cells from a tumor, where 8 to 15 different T cell receptor (TCR) clonotypes make up at least 50% of the TCR frequency in the population with greater and lesser number of clonotypes and population percentages contemplated According to some embodiments, a pharmaceutical composition enriched in tumor reactive T cells comprises an oligoclonal population of tumor infiltrating lymphocytes comprising CD4+ and CD8+ T cells from a tumor, where 8 to 15 different T cell receptor (TCR) clonotypes make up at least 50% of the TCR frequency in the population with greater and lesser number of clonotypes and population percentages contemplated According to some embodiments, 9 to 12 different TCR clonotypes make up at least 50% of the TCR frequency in the population.

In various embodiments, the TCR clonotypes exhibit reactivity to at least one CD4 antigen and at least one CD8 antigen. In some of any of the provided embodiments, the TCR clonotypes exhibit reactivity for 2 to 100 different peptide antigens. In some of any of the provided embodiments, the TCR clonotypes exhibit reactivity for 10 to 40 different peptide antigens. In some of any of the provided embodiments, the TCR clonotypes exhibit reactivity for 2 to 6 different peptide antigens. In some of any of the provided embodiments, the TCR clonotypes exhibit reactivity for 2 to 4 peptide antigens. In some of any of the provided embodiments, the TCR clonotypes exhibit reactivity for 2 peptide antigens. In some of any of the provided embodiments, the TCR clonotypes exhibit reactivity for one CD8 antigen and one CD4 antigen.

Provided herein is a pharmaceutical composition enriched in tumor reactive T cells, the pharmaceutical composition comprising a multiclonal population of tumor infiltrating lymphocytes comprising CD4+ and CD8+ T cells from a tumor, wherein 10 to 100 different T cell receptor (TCR) clonotypes are present in the population. In some of any of the provided embodiments, the TCR clonotypes exhibit reactivity for 10 to 40 different peptide antigens.

Provided herein is a pharmaceutical composition enriched in tumor reactive T cells, the pharmaceutical composition comprising a multiclonal population of tumor infiltrating lymphocytes comprising CD4+ and CD8+ T cells from a tumor, wherein 20 to 100 different T cell receptor (TCR) clonotypes are present in the population. In some of any of the provided embodiments, 20 to 60 different TCR clonotypes are present in the population.

Provided herein is a pharmaceutical composition enriched in tumor reactive T cells, the pharmaceutical composition comprising a multiclonal population of tumor infiltrating lymphocytes comprising CD4+ and CD8+ T cells from a tumor, wherein the top 40 TCR clonotypes make up at least 75% of the TCR frequency in the population. In some of any of the provided embodiments, at least 90% of the cells in the population are CD3+ T cells. In some of any of the provided embodiments, the TCR clonotypes exhibit reactivity for at least one CD8 antigen and at least one CD4 antigen.

In some of any of the provided embodiments, at least 20% of the CD8+ T cells and/or at least 20% of the CD4+ T cells in the composition exhibit neoantigen reactivity.

Various embodiments provide a pharmaceutical T lymphocyte infiltrating (TIL) composition enriched in tumor reactive T cells, comprising tumor infiltrating lymphocytes comprising CD4+ and CD8+ T cells from a tumor, wherein at least 90% of cells in the composition are CD3+ T cells and wherein at least 20% of the CD8+ T cells and/or at least 20% of the CD4+ T cells in the composition exhibit neoantigen reactivity. In some of any of the provided embodiments, at least 40% of the CD8+ T cells and/or at least 30% of the CD4+ T cells in the composition exhibit neoantigen reactivity.

In one or more embodiments, at least 25% of the CD8+ T cells and/or at least 20% of the CD4+ T cells in the composition exhibit neoantigen reactivity. In some of any of the provided embodiments, at least 30% of the CD8+ T cells and/or at least 20% of the CD4+ T cells in the composition exhibit neoantigen reactivity. In some of any of the provided embodiments, neoantigen reactivity is determined following co-culture with peptide loaded antigen presenting cells and characterized by one or more of IFN-γ production or degranulation. In some embodiments, degranulation is determined based on CD107 expression.

In some of any of the provided embodiments, neoantigen reactivity is determined in a co-culture assay with autologous APCs and neoantigenic peptides (e.g., as described in Example 2) by one or more of upregulation of CD134 and CD137, IFN-γ production, TNF-alpha production, granzyme B production or degranulation. In some embodiments, degranulation is determined based on CD107 expression. In some of any of the provided embodiments, the TIL composition is characterized by at least a 1.5-fold increased percentage of cells positive for CD134 and CD137 compared to a bulk TIL population in a co-culture assay with autologous APCs and neoantigenic peptides. In some embodiments, the TIL composition is characterized by at least a 2-fold, at least a 3-fold or at least a 4-fold increase in CD134 and CD137 positive cells compared to a bulk TIL population. In some of any of the provided embodiments, greater than 30% of the cells in the TIL composition are positive for CD134 and CD137 in a co-culture assay with peptide loaded autologous APCs, optionally greater than about 35%, greater than about 40%, or greater than about 45% of cells are positive for CD134 and CD137.

In some of any of the provided embodiments, greater than 48% of the cells in the TIL composition are positive for CD134 and CD137 in a co-culture assay with autologous APCs and neoantigenic peptides. In some embodiments, greater than about 50%, greater than about 60%, or greater than about 70% of cells are positive for CD134 and CD137 in a co-culture assay with autologous APCs and neoantigenic peptides.

In some of any of the provided embodiments, the TIL composition is characterized by at least one of the following criteria in an in vitro co-culture assay with peptide loaded antologous APC, i) IFN-γ production that is greater than 1,000 pg/mL; (ii) TNF-alpha production that is greater than 100 pg/mL; (iii) greater than 10% CD107a+ cells; and iv) granzyme B production that is greater than 10,000 pg/mL.

In some of any of the provided embodiments, the TIL composition is characterized by at least one of the following in an in vitro co-culture assay with autologous APC and neoantigenic peptides: i) IFN-γ production that is greater than 100,000 pg/mL; (ii) TNF-alpha production that is greater than 250 pg/mL; (iii) greater than 10% CD107a+ cells; and iv) granzyme B production that is greater than 50,000 pg/mL.

Provided herein is a pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following criteria in an in vitro co-culture assay with peptide loaded autologous APC: i) IFN-γ production that is greater than 1,000 pg/mL; (ii) TNF-alpha production that is greater than 100 pg/mL; (iii) greater than 10% CD107a+ cells; and (iv) granzyme B production that is greater than 10,000 pg/mL.

Provided herein is a pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following in an in vitro co-culture assay: i) IFN-γ production that is greater than 100,000 pg/mL; (ii) TNF-alpha production that is greater than 250 pg/mL; (iii) greater than 10% CD107a+ cells; and (iv) granzyme B production that is greater than 50,000 pg/mL.

In some of any of the provided embodiments, the TIL composition is characterized by at least two of (i)-(iv). In some of any of the provided embodiments, the TIL composition is characterized by at least three of (i)-(iv). In some of any of the provided embodiments, the TIL composition is characterized by (i)-(iv).

In some of any of the provided embodiments, the TIL composition is characterized by IFN-γ production that is greater than 2,500 pg/mL, greater than 5,000 pg/mL, greater than 10,000 pg/mL, greater than 25,000 pg/mL., greater than 50,000 pg/mL, greater than 100,000 pg/mL, greater than 200,000 pg/mL, greater than 250,000 pg/mL, greater than 500,000 pg/mL, or greater than 1,000,000 pg/mL. In some of any of the provided embodiments, the TIL composition is characterized by IFN-γ production that is greater than 250,000 pg/mL, greater than 500,000 pg/mL, or greater than 1,000,000 pg/mL. In some of any of the provided embodiments, the TIL composition is characterized by IFN-γ production that is greater than 250,000 pg/mL, greater than 500,000 pg/mL, or greater than 1,000,000 pg/mL. In some of any of the provided embodiments, wherein the TIL composition is characterized by TNF-alpha production that is greater than 200 pg/mL, greater than 500 pg/mL., greater than 1000 pg/mL, or greater than 2000 pg/mL. In some of any of the provided embodiments, the TIL composition is characterized by TNF-alpha production that is greater 500 pg/mL, greater than 1000 pg/mL, or greater than 2000 pg/mL.

In some of any of the provided embodiments, the TIL composition is characterized by greater than 15% CD107a+ cells, greater than 20% CD107a cells, or greater than 25% CD107a+ cells. In some of any of the provided embodiments, the TIL composition is characterized by granzyme B production that is greater than 15,000 pg/mL, greater than 25,000 pg/mL, greater than 50,000 pg/mL., greater than 100,000 pg/mL, greater than 200,000 pg/mL, greater than 300,000 pg/mL, greater than 400,000 pg/mL or greater than 500,000 pg/mL. In some of any of the provided embodiments, the TIL composition is characterized by granzyme B production that is greater than 200,000 pg/mL, greater than 300,000 pg/mL, greater than 400,000 pg/mL or greater than 500,000 pg/mL.

In some of any of the provided embodiments, following co-culture with peptide loaded antigen presenting cells, the TIL composition is characterized by at least one of the following: i) IFN-γ secretion in the supernatant that is 50-fold or higher than a bulk TIL composition that is not enriched for tumor reactive T cells; ii) TNF-α secretion in the supernatant that is 300-fold or higher than a bulk TIL composition that is not enriched for tumor reactive T cells; or iii) granzyme B secretion in the supernatant that is 15-fold or higher than a bulk TIL composition that is not enriched for tumor reactive T cells.

In some of any of the provided embodiments, the TIL composition is characterized by at least one of the following criteria in and/or determined by an in vitro co-culture assay: i) IFN-γ that is 50-fold or higher than a bulk TIL composition that is not enriched for tumor reactive T cells; ii) TNF-α that is 300-fold or higher than a bulk TIL composition that is not enriched for tumor reactive T cells; or iii) granzyme B that is 15-fold or higher than a bulk TIL composition that is not enriched for tumor reactive T cells.

In many embodiments, a pharmaceutical T lymphocyte infiltrating (TIL) composition enriched in tumor reactive T cells, comprises a CD4+ and CD8+ T cells from a tumor, wherein at least 90% of cells in the composition are CD3+ T cells and wherein, following co-culture with peptide loaded antigen presenting cells, the TIL composition is characterized by at least one of the following criteria: i) IFN-γ secretion in the supernatant that is 50-fold or higher than a bulk TIL composition that is not enriched for tumor reactive T cells; ii) TNF-α secretion in the supernatant that is 300-fold or higher than a bulk TIL composition that is not enriched for tumor reactive T cells; or iii) granzyme B secretion in the supernatant that is 15-fold or higher from a bulk TIL composition that is not enriched for tumor reactive T cells.

In many embodiments, a pharmaceutical T lymphocyte infiltrating (TIL) composition enriched in tumor reactive T cells, comprises a CD4+ and CD8+ T cells from a tumor, wherein at least 90% of cells in the composition are CD3+ T cells and wherein the TIL composition is characterized by at least one of the following criteria in and/or determined by an in vitro co-culture assay: i) IFN-γ that is 50-fold or higher than a bulk TIL composition that is not enriched for tumor reactive T cells; ii) TNF-α that is 300-fold or higher than a bulk TIL composition that is not enriched for tumor reactive T cells; or iii) granzyme B that is 15-fold or higher from a bulk TIL composition that is not enriched for tumor reactive T cells.

In some of any of the provided embodiments, the TIL composition is characterized by criteria (i) and (ii). In some of any of the provided embodiments, the TIL composition is characterized by criteria (i) and (iii). In some of any of the provided embodiments, the TIL composition is characterized by criteria (ii) and (iii). In some of any of the provided embodiments, the TIL composition is characterized by criteria (i), (ii) and (iii).

In some of any of the provided embodiments, the composition is characterized by a greater number of CD4+ T cells than CD8+ T cells. In some of any of the provided embodiments, a ratio of CD4+ T cells to CD8+ T cells in the composition is between 5:1 to 1:5. In some of any of the provided embodiments, a ratio of CD4+ T cells to CD8+ T cells in the composition is between 5:1 to 50:1, between 5:1 to 25:1, between 5:1 to 20:1, between 5:1 to 15:1, between 5:1 to 10:1, between 10:1 to 50:1, between 10:1 to 25:1, between 10:1 to 20:1, between 10:1 to 15:1, between 15:1 to 50:1, between 15:1 to 25:1, between 15:1 to 20:1, between 20:1 to 50:1, between 20:1 to 25:1 or between 25:1 to 50:1. In some of any of the provided embodiments, a ratio of CD4+ T cells to CD8+ T cells in the composition is at or about 10:1 to 25:1. In some embodiments, the ration of CD4+ T cells to CD8+ T cells is at or about 20:1.

In some of any of the provided embodiments, greater than 50% of the CD3+ T cells, optionally greater than 50% of the CD4 and CD8+ T cells, express markers of an effector memory phenotype. According to various embodiments, greater than 75% of the CD3+ T cells, optionally greater than 75% of the CD4 and CD8+ T cells, express markers of an effector memory phenotype. In some of any of the provided embodiments, greater than 80% of the CD3+ T cells, optionally greater than 80% of the CD4 and CD8+ T cells, express markers of an effector memory phenotype. In some of any of the provided embodiments, greater than 85% of the CD3+ T cells, optionally greater than 85% of the CD4 and CD8+ T cells, express markers of an effector memory phenotype. In some of any of the provided embodiments, greater than 90% of the CD3+ T cells, optionally greater than 90% of the CD4 and CD8+ T cells, express markers of an effector memory phenotype. In some of any of the provided embodiments, the effector memory phenotype is characterized by surface marker expression of one or more of CD45RO+, CD45RA−, CD62L−, CCR7−, CD28− and CD27−. In some of any of the provided embodiments, the effector memory phenotype is characterized by surface marker expression CD45RO+, CD45RA−, CD62L−, and CCR7−. In some of any of the provided embodiments, the effector memory phenotype is characterized by surface marker expression CD45RO+, CD45RA−, CD62L−, CCR7−, CD28− and CD27−. In some of any of the provided embodiments, the effector memory phenotype is characterized by surface marker expression CD45RA⁻ and CCR7⁻. In some of any of the provided embodiments, greater than 95% of the CD4+ and CD8+ T cells in the composition are PD-1−. In some of any of the provided embodiments, greater than 80% of the CD 4+ and CD8+ T cells in the composition LAG3−.

In some of any of the provided embodiments, the number of cells in the composition, or of viable cells thereof, is at least 2×10⁷ cells. In some of any of the provided embodiments, the number of cells in the composition, or of viable cells thereof, is between at or about 2×10⁷ cells and 20×10⁹ cells, 2×10⁷ cells and 10×10⁹ cells, 2×10⁷ cells and 2×10⁹ cells, 2×10⁷ cells and 2×10⁸ cells, 2×10⁸ cells and 20×10⁹ cells, 2×10⁸ cells and 10×10⁹ cells, 2×10⁸ cells and 2×10⁹ cells, 2×10⁹ cells and 20×10⁹ cells, 2×10⁹ cells and 10×10⁹ cells, or 10×10⁹ cells and 20×10⁹ cells, each inclusive.

In some of any of the provided embodiments, the pharmaceutical composition is for treatment of a patient's tumor. In some of any of the provided embodiments, the tumor is a colorectal cancer (CRC) tumor. In some of any of the provided embodiments, the tumor is a colorectal cancer (CRC) tumor, a melanoma tumor, a non-small cell lung cancer (NSCLC) tumor, or an ovarian cancer tumor. In some of any of the provided embodiments, the tumor is from a human subject. In some of any of the provided embodiments, the pharmaceutical composition is for autologous adoptive therapy to the human subject. In some of any of the provided compositions, comprising a pharmaceutically acceptable excipient. Provided herein is any of the provided compositions, comprising a cryoprotectant.

In some of any of the provided embodiments, the composition is a liquid composition. In some of any of the provided embodiments, the composition had been frozen and thawed. In some of any of the provided embodiments, the volume of the composition is between 1 mL and 500 mL. In some of any of the provided embodiments, the composition is frozen.

Many embodiments provide a pharmaceutical T lymphocyte infiltrating (TIL) composition enriched in tumor reactive T cells for treatment of a patient's tumor, the pharmaceutical composition comprising a multiclonal population of T cells comprising CD4+ and CD8+ T cells from the patient's tumor, wherein the population is enriched in at least 10 different T cell receptor (TCR) clonotypes each with a frequency in the population of at least 1.0%; and wherein at least 90% of the cells in the composition are CD3+ T cells. Further embodiments provide a pharmaceutical T lymphocyte infiltrating (TIL) composition enriched in tumor reactive T cells for treatment of a patient's tumor, the pharmaceutical composition comprising a multiclonal population of T cells comprising CD4+ and CD8+ T cells from the patient's tumor, wherein the population is enriched in at least 10 different T cell receptor (TCR) clonotypes each with a frequency in the population of at least 2.0%; and wherein at least 90% of the cells in the composition are CD3+ T cells. Other embodiments provide a pharmaceutical T lymphocyte infiltrating (TIL) composition enriched in tumor reactive T cells for treatment of a patient's tumor, the pharmaceutical composition comprising an oligoclonal population of T cells comprising CD4+ and CD8+ T cells from the patient's tumor, wherein the population is enriched in at least 10 different T cell receptor (TCR) clonotypes each with a frequency in the population of at least 2.0%; and wherein at least 90% of the cells in the composition are CD3+ T cells. Also provided herein is a pharmaceutical T lymphocyte infiltrating (TIL) composition enriched in tumor reactive T cells for treatment of a patient's tumor, the pharmaceutical composition comprising an oligoclonal population of T cells comprising CD4+ and CD8+ T cells from the patient's tumor, wherein the population comprises at least 10 different T cell receptor (TCR) clonotypes each with a frequency in the population of at least 1.0%; and wherein at least 90% of the cells in the composition are CD3+ T cells.

In some of any of the provided embodiments, the TIL composition is produced by an ex vivo method comprising expansion of tumor-reactive T cells from a donor subject that have been co-cultured with autologous antigen presenting cells and peptide neoantigens.

In some of any of the provided embodiments, the TIL composition is produced by a method comprising: a. providing dissociated tumor cells from a tumor obtained from a donor subject, wherein the dissociated tumor cells are a first population of T cells that comprise CD4+ and CD8+ T cells; b. culturing the first population of T cells with recombinant IL-2 added at a concentration between 3000 IU/mL and 6000 IU/mL, inclusive, for 14 to 28 days to produce a second population of T cells; c. co-culturing the second population of T cells for 12 to 48 hours with autologous antigen presenting cells (APCs) with recombinant IL-2 added at a concentration of 100IU/mL to 1000 IU/mL to produce a third population of T cells, wherein the APCs are loaded with a pool of peptide neoantigens (i.e., neoantigenic peptides) from the tumor, wherein each peptide is 13-40 amino acids in length and is loaded at a concentration of 100 ng/ml per peptide, and wherein the ratio of the second population of T cells to APCs is 2:1 to 10:1; d. selecting cells from the third population of T cells that are surface positive for CD134 and/or CD137 to produce a fourth population of T cells; and e. expanding tumor infiltrating lymphocytes (TILs) by incubating the fourth population of T cells with irradiated human peripheral blood mononuclear cells (iPBMCs) at a ratio of 100 to 500 IPBMC to cells of the fourth population of T cells with recombinant IL-2 added at a concentration between 3000 IU/mL and 6000 IU/mL, inclusive, and 10 to 50 ng/ml anti-CD3 antibody (OKT3) for 12 to 16 days to produce a therapeutic composition of TILs enriched in tumor reactive cells.

Provided herein is a frozen composition comprising the pharmaceutical composition of any of the provided compositions and a cryoprotectant

Provided herein is a method of producing a T lymphocyte infiltrating (TIL) composition enriched in tumor reactive T cells, the method comprising: a. providing dissociated tumor cells from a tumor obtained from a donor subject, wherein the dissociated tumor cells are a first population of T cells that comprise CD4+ and CD8+ T cells; b. culturing the first population of T cells with recombinant IL-2 added at a concentration between about 3000 IU/mL and 6000 IU/mL, inclusive, for about 14 to 28 days to produce a second population of T cells; c. co-culturing the second population of T cells for about 12 to 48 hours with autologous antigen presenting cells (APCs) with recombinant IL-2 added at a concentration of 100 IU/mL to 1000 IU/mL to produce a third population of T cells, wherein the APCs are loaded with a pool of peptide neoantigens (i.e., neoantigenic peptides) from the tumor, wherein each peptide is 13-40 amino acids in length and is loaded at a concentration of 100 ng/ml per peptide, and wherein the ratio of the second population of T cells to APCs is about 2:1 to 10:1; d. selecting cells from the third population of T cells that are surface positive for CD134 and/or CD137 to produce a fourth population of T cells; and e. expanding tumor infiltrating lymphocytes (TILs) by incubating the fourth population of T cells with irradiated human peripheral blood mononuclear cells (iPBMCs) at a ratio of about 100 to 500 iPBMC to cells of the fourth population of T cells with recombinant IL-2 added at a concentration between about 3000 IU/mL and 6000 IU/mL, inclusive, and 10 to 50 ng/ml anti-CD3 antibody (OKT3) for 12 to 16 days to produce a therapeutic composition of TILs enriched in tumor reactive cells.

Provided herein is a method of treating a subject having a cancer, the method comprising administering to a subject having a tumor a therapeutic dose of any of the provided compositions. In some of any of the provided embodiments, the therapeutically effective dose is between about 1×10⁹ and 10×10⁹ T cells. In some of any of the provided embodiments, the therapeutically effective dose is from more than 1 million to less than 100 million T cells per kilogram of body weight. In some of any of the provided embodiments, the therapeutically effective dose is from more than 1 million to less than 10 million T cells per kilogram of body weight. In some of any of the provided embodiments, the therapeutically effective dose is from at or about 10 million to at or about 50 million T cells per kilogram of body weight. In some of any of the provided embodiments, the cells of the therapeutic composition are autologous to the subject.

### BRIEF DESCRIPTION OF THE DRAWINGS

FIG. 1 depicts an embodiment of a process for preparing enriched tumor reactive TILs involving unbiased mutation calling with peptides generated against large breadth (up to 200) relevant tumor neoantigens, co-culture of TILs prepared from dissociated tumor cells of a CRC patient with autologous antigen presenting cells to optimize peptide-based antigen presentation and capture greatest TCR diversity within TIL population, and selection sorting of cells with validated markers such as CD134 and/or CD137 to enrich for both CD4+ and CD8+ TILs with the highest tumor reactivity.

FIG. 2A depicts tumor mutational burden (TMB) across multiple indications. The number of tumor specific neoantigens (i.e., neoantigenic peptides) identified by whole exome and transcriptome (RNAseq) of a patient's tumor and non-tumor (peripheral blood leukocytes) tissues (n=15) are shown for each indication. Horizontal lines depict the mean TMB within each tumor type.

FIG. 2B depicts preREP yield across multiple indications as demonstrated by total live cells of TIL from fresh tumor fragments or single cell suspensions (n=31). Each dot represents an individual patient, vertical lines represent the standard deviation.

FIG. 3 shows flow cytometry plots of TIL stained for CD134 and CD137 after coculture with APCs unpulsed or pulsed with a pool of 190 peptides containing predicted mutations. Sorting gates are outlined in solid line or with hash marks. TIL expressing CD134 and/or CD137 following coculture with peptide loaded APC were sorted and expanded as the selected TIL fraction (3.93%, right panel). Sorting gates were set on viable lymphocytes expressing CD4 and/or CD8 prior to defining the CD134 and CD137 gates. The percentage of CD4+ or CD8+ cells within the sorting gates are shown in the top right corner of each flow plot in blue.

FIG. 4A depicts the fold expansion of sorted T populations CD134 and/or CD137 selected TIL) at days 10 and 14 of REP. The fold change was calculated by dividing the total number of live cells Days 1, 10 and 14 by the number of live cells seeded at Day 1.

FIG. 4B depicts expansion of selected TILS after a 14-day REP (n=8). Each dot represents an individual patient, vertical lines represent the standard deviation.

FIG. 5 depicts the phenotype of selected TIL. FIG. 5 (left panel) depicts the % of CD3+ T cell subsets defined by CD4 and CD8 expression at end of REP in bulk and select TIL from CRC (n=3), melanoma (n=1) and ovarian (n=1), FIG. 5 (right panel) depicts T cell memory populations defined based on expression of CD45RA and CCR7 within the CD4 and CD8 subsets. Each circle represents an individual patient.

FIG. 6A depicts a stacked box plot showing the frequency of TCR clonotypes in selected TILs, bystander cells (negative fraction, unselected) and bulk TILs as determined by single cell TCR sequencing. The top 18 most frequent clonotypes are shown by solid boxes while all other TCR clonotypes are identified by scRNA-seq for each sample are shown in the single hashed bar.

FIG. 6B depicts reduced TCR diversity in selected TILs relative to bulk TILs from an ovarian tumor at the end of REP. FIG. 6B (left panel) depicts diversity of TCR clonotypes as assessed by single cell RNA sequencing on bulk, selected and negative selected TIL. FIG. 6B (right panel) depicts abundance of TCR clonotypes as assessed by single cell RNA sequencing on bulk, selected and negative selected TIL. Each color block represents a unique TCR and colored lines connecting samples indicate shared TCR clonotypes. The frequency of the Top40 most abundant clonotypes across all samples are displayed.

FIG. 7A depicts CD134+CD137+ expression in bulk and selected TIL from CRC (n=3) and melanoma (n=1) as assessed by co-culture of selected TILs and bulk TILs at the end of REP with peptide pulsed or un-pulsed APCs at a ratio of 5:1.

FIG. 7B depicts intracellular IFN-γ production in CD4 T cells or CD8 T cells of bulk TILs (far left for each condition), selected TIL (middle bar for each condition) and negative fraction bystander cells (far right for each condition) after co-culture at an effector: target ratio of 5:1 with unloaded (DMSO) or peptide loaded APC (B cells). Error bars represent standard error of the mean of triplicate wells. ****P<0.0001, Two-way ANOVA.

FIG. 7C depicts enrichment for neoantigen (i.e., neoantigenic peptide) reactivity of selected TILs compared to bulk TIL after REP as assessed by T cell activation (CD134+CD137+) and intracellular cytokine production of IFN-γ, FIG. 7C (left panel) depicts selected TILs are enriched for neoantigen reactivity as shown by fold change increase in CD134+CD137+ in selected TIL relative to bulk TILs. FIG. 7C (right panel) depicts selected TILs are enriched for neoantigen reactivity as shown by fold change in intracellular IFN-γ in selected TIL relative to bulk TILs.

FIG. 8A depicts cytokine secretion of bulk TIL (far left for each condition), selected TIL (middle bar for each condition) and negative fraction bystander cells (far right for each condition) after co-culture for 24 hours at an effector:target (E:T) of 5:1 with unloaded (DMSO) or peptide loaded APC (B cells). Error bars depict standard error of the mean of triplicate wells. *****P<0.0001, Two-way ANOVA.

FIG. 8B depicts increased cytokine secretion from selected TILs relative to bulk TILs in response to neoantigen (i.e., neoantigenic peptide) specific stimulation as quantified by multiplex cytometric bead array for IFN-γ and TNFα levels in supernatants harvested 24 hours after co-culture with peptide pulsed or un-pulsed APCs. Dots represent technical replicates; error bars display standard deviation of triplicates.

FIG. 9A depicts CD107a expression of bulk TIL (far left for each condition), selected TIL (middle bar for each condition) and negative fraction bystander cells (far right for each condition) after co-culture at an effector: target ratio of 5:1 with unloaded (DMSO) or peptide loaded APC (B cells). Error bars depict standard error of the mean of triplicate wells. ****P<0.0001, Two-way ANOVA.

FIG. 9B depicts increased CD107a expression in CD4+ and CD8+ subsets for selected TILs relative to bulk TILs in response to neoantigen (i.e., neoantigenic peptide) specific stimulation after co-culture with peptide pulsed or un-pulsed APCs. Error bars display standard deviation of triplicates.

FIG. 9C depicts Granzyme B expression in CD8 T cells of bulk TIL (far left for each condition), selected TIL (middle bar for each condition) and negative fraction bystander cells (far right for each condition) after co-culture at an effector: target ratio of 5:1 with unloaded (DMSO) or peptide loaded APC (B cells). Error bars represent standard error of the mean of triplicate wells. *** P<0.0005, Two-way ANOVA.

FIG. 9D depicts increased Granzyme B expression in CD8+ subsets for selected TILs relative to bulk TILs in response to neoantigen (i.e., neoantigenic peptide) specific stimulation after co-culture with peptide pulsed or un-pulsed APCs. Dots represent technical replicates and error bars display standard deviation of triplicates.

FIGS. 10A-10B show that selected TIL are functional in response to non-specific polyclonal stimulation. Bulk and selected TIL from CRC (n=3) and melanoma (n=1) were stimulated overnight with soluble CD3/CD28 activator (CD134+CD137+), or for 5 hours with PMA/Ionomycin (IFN-γ, TNFα and CD107a) and cells assessed for CD134+CD137+, IFN-γ, TNFα and CD107a expression on (FIG. 10A) CD4+ and (FIG. 10B) CD8+ cells by flow cytometry. Each circle represents an individual sample with bulk TIL annotated by open white circles and selected TIL by black circles. Horizontal lines depict the mean percent expression of each population.

FIGS. 11A-11B depicts results of deconvolution analysis of peptide reactivity as determined by CD4+ T cells (FIG. 8A) and CD8+ T cells (FIG. 8B) in the selected TILs that were IFN-γ+ following coculture of TILs with unloaded APCs or APCs loaded with the 190 peptides or a smart peptide pool of 13-14 peptides.

FIG. 12 depicts an exemplary process for generating TIL with enriched reactivity to mutations expressed in neoantigens from patient material. TIL expansion from a digested colorectal cancer (CRC) and gastric tumor sample are shown in FIG. 13.

FIG. 14A shows upregulation of CD134 (OX40)/CD137 (4-1BB), as well as IFNγ, TNFα, and Granzyme B expression in cellular supernatants. An exemplary gating strategy for sorting of neoantigen peptide reactive (+/+) and non-reactive (−/−) TIL is shown in FIG. 14B. FIG. 14C shows fold increase in sorted samples following a Rapid Expansion Protocol (REP).

A representative sample of neoantigen reactive (+/+) and non-reactive (−/−) TIL following REP is shown in FIG. 15A. Reactivity is shown in FIG. 15B and was measured by CD137 (4-1BB) and CD134 (OX40) upregulation following co-culture. FIG. 15C depicts reactivity as measured by upregulation of Granzyme B, IFNγ, and TNFα secretion in supernatant in an ELLA assay. Peptides were further screened for individual reactivity by upregulation of IFNγ secretion as shown in FIG. 15D.

### DETAILED DESCRIPTION

Various embodiments of the invention provide compositions of tumor infiltrating lymphocytes (TILs) enriched in tumor reactive cells in which the cells are characterized by diversity in TCR clonality and exhibit robust neoantigen reactivity to neoantigen peptides, particularly compared to TIL compositions with less TCR clonal diversity. Related embodiments provide methods for manufacturing tumor infiltrating lymphocytes (TILs) enriched in such tumor reactive cells.

The provided compositions and methods relate to producing a T cell therapy reactive to tumor-associated antigens, such as neoantigens (i.e., neoantigenic peptides). Cancer cells accumulate lots of different DNA mutations as part of the tumorigenic process. These mutations can cause amino acid changes in protein coding regions. For a mutation to be recognized by the immune system the protein needs to be processed intracellularly and presented on the surface with the Major Histocompatibility Complex (MHC). Peptide neoantigens (also referred to herein as neoantigenic peptides, neoepitopes or peptide neoepitopes) are the mutant peptides presented by the MHC complex that can be recognized by a T-cell via TCR binding. In order for the immune system to recognize the mutation, it must be expressed on the surface of the cancer cell via the MHC complex and the T cell must have a TCR that recognizes the mutated peptide. These neoantigens may be presented by MHC class I and MHC class II, and are recognized by CD8+ and CD4+ T cells respectively.

In particular embodiments of the provided compositions and methods, the tumor reactive cells express a T cell receptor (TCR) able to recognize the neoantigens (i.e., neoantigenic peptides).

In some embodiments, a “T cell receptor” or “TCR” is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a T cells (or T lymphocytes) where it is generally responsible for recognizing antigens bound to major histocompatibility complex (MHC) molecules.

In some aspects, the reactive T cells are tumor-reactive T cells that recognize a cancer neoantigen (i.e., neoantigenic peptide). The majority of neoantigens arise from passenger mutations, meaning they do not infer any growth advantage to the cancer cell. A smaller number of mutations actively promote tumor growth, these are known as driver mutations. Passenger mutations are likely to give rise to neoantigens that are unique to each patient and may be present in a subset of all cancer cells. Driver mutations give rise to neoantigens that are likely to be present in all the tumor cells of an individual and potentially shared. In some embodiments of the provided method, the population of T cells contain tumor-reactive T cells that can recognize neoantigens containing passenger and/or driver mutations.

In some aspects, neoantigens (i.e., neoantigenic peptides) are ideal targets for immunotherapies because they represent disease-specific targets. For example, such antigens generally are not present in the body before the cancer developed and are truly cancer specific, not expressed on normal cells and are not subjected to off target immune toxicity. Thus, the unique repertoire of neoantigens specific to the patient can elicit a strong immune response specific to the cancer cells, avoiding normal cells. This is an advantage over other cell therapy targets that may not be disease-specific targets, since even low levels of target antigen on normal cells can lead to severe fatal autoimmune toxicity in the context of engineered therapies that target common antigens. For example, an anti-MAGE-A3-TCR program in melanoma patients was halted due to study related deaths attributed to cross reactivity with a similar target MAGE-A12, which is expressed at a low level in the brain. A significant challenge in cancer immunotherapy has been the identification of cancer targets.

Recent clinical studies have demonstrated that T cells isolated from surgically resected tumors possess TCRs that recognize neoantigens (i.e., neoantigenic peptides), and expanding these neoantigen reactive TIL populations and re-infusing them into the patient can in some cases result in a dramatic clinical benefit. This personalized therapy has generated remarkable clinical responses in certain patients with common epithelial tumors.

TIL therapy has proved to be most effective in melanoma. Additionally, enhancing tumor reactivity by selective expansion of individual TIL subpopulations, screened for neoantigen reactivity, has demonstrated some success in breast cancer (Zacharakis et al., 2022, J. Clin. Oncol. JCO2102170). However, while other solid tumors such as colorectal cancer (CRC) have been shown to contain neoantigen reactive TIL (Parkhurst et al., 2019 Cancer Discov. 9:1022-1035), the ability to selectively enrich these cells has been challenging. Here we demonstrate the generation of TIL compositions with improved tumor reactivity, including for solid tumors such as CRC. In some aspects, provided embodiments demonstrate enrichment of tumor reactive cells by an ex vivo process that utilizes tumor-specific mutation containing peptides to select neoantigen reactive TIL by fluorescence-activated single cell sorting (FACS). Such a composition when produced from a CRC patient tumor can result in a neoantigen targeted selected TIL product in CRC.

Existing methods for obtaining and generating tumor-reactive T cells are not entirely satisfactory. For example, direct isolation of tumor-reactive T cells from a subject without expansion is not feasible because therapeutically effective amounts of such cells cannot be obtained. As an alternative, attempts have been made to identify TCRs specific to a desired neoantigen (i.e., neoantigenic peptides) for recombinant engineering of the TCR into T cells for use in adoptive cell therapy methods. Such approaches, however, produce only a single TCR against a specific neoantigen and thereby lack diversity to recognize a broader repertoire of multiple tumor-specific mutations. Other methods involve bulk expansion of T cells from a tumor source, which has the risk of expanding T cells that are not reactive to a tumor antigen and/or that may include a number of bystander cells that could exhibit inhibitory activity. For example, tumor regulatory T cells (Tregs) are a subpopulation of CD4+ T cells, which specialize in suppressing immune responses and could limit reactivity of a T cell product. These further approaches that have sought to expand tumor-reactive T cells ex vivo are not selective such that non-reactive T cells in the culture may preferentially expand over reactive T cells resulting in a final product that lacks satisfactory reactivity and/or in which the number of tumor-reactive T cells remains insufficient. Methods to produce tumor-reactive T cells for therapy are needed.

Various embodiments of methods of producing tumor infiltrating lymphocytes (TILs) enriched in such tumor reactive cells involve the ex vivo expansion and production of a T cell therapeutic composition, particularly for use in connection with treating cancer such as melanoma colorectal cancer (CRC) breast, and liver cancer. In some embodiments, the method of manufacturing involves the growth and manipulation of patient cells outside of the body. In particular embodiments, the methods relate to methods for expanding T cells containing an endogenous TCR specific to a tumor-associated antigen (hereinafter “tumor reactive T cells”). For purposes of this disclosure, reference to tumor reactive T cells includes T cells that exhibit reactivity to a tumor antigen, as evidenced by the ability of the TIL to produce or secrete IFNy or TNFα or express a factor involved or related to cytotoxic killing activity (e.g., Granzyme B or a degranulation factor such as CD107) following exposure to APCs presenting neoantigenic peptides. In some aspects, the frequency of these cells can be low and in order to expand these cells to a therapeutic dose ex vivo methods for enrichment and expansion are necessary, as provided by the present embodiments.

Thus, among embodiments of TIL compositions provided are TIL compositions that exhibit substantial tumor reactivity activity, including degranulation and ability to express more IFN-gamma and TNF-alpha, in response to APCs (e.g., DCs or B cells) presenting neoantigenic peptides. This functional activity is highly preserved even after cryopreservation and thawing of the TIL compositions. The marked increases in cytolytic enzymes, as well as more robust activation phenotypes, underpin the enhanced capacity of the expanded TIL compositions to induce apoptosis of tumor targets. The marked tumor reactivity also supports the utility of the provided TIL composition as a therapy.

The provided methods results in a product containing tumor reactive T cells that can target many mutations and/or that are enriched in a multiclonal TCR TIL population that are reactive to different tumor antigens. In some embodiments, the multiclonal TCR TIL population is an oligoclonal TCR TIL population. Therefore, the provided methods results in a product containing tumor reactive T cells that can target many mutations and/or that are enriched in an oligoclonal TCR TIL population that are reactive to different tumor antigens. Thus, such tumor reactive T cells offer advantages compared to existing methods in which cells are transduced to express a single neoepitope reactive TCR, or in which TILs are expanded in bulk or exhibit limited multiclonality and/or oligoclonality.

In some embodiments of the provided methods a source of potential tumor peptides is used to identify TCRs that are reactive to neoantigens in a process that includes expansion of the T cells reactive to the tumor neoantigenic peptides. Provided methods include ex vivo co-culture methods in which a population of T cells that have been expanded from T cells present in or from a tumor sample is incubated in the presence of antigen-presenting cells that have been contacted with, or made to present, the neoantigenic peptides. In particular aspects, the T cells and antigen-presenting cells are autologous to the tumor-bearing subject from which the peptides were identified. The provided methods further include steps to separate, enrich for and/or select for tumor-reactive T cells from the co-culture prior to or in connection with their further ex vivo expansion.

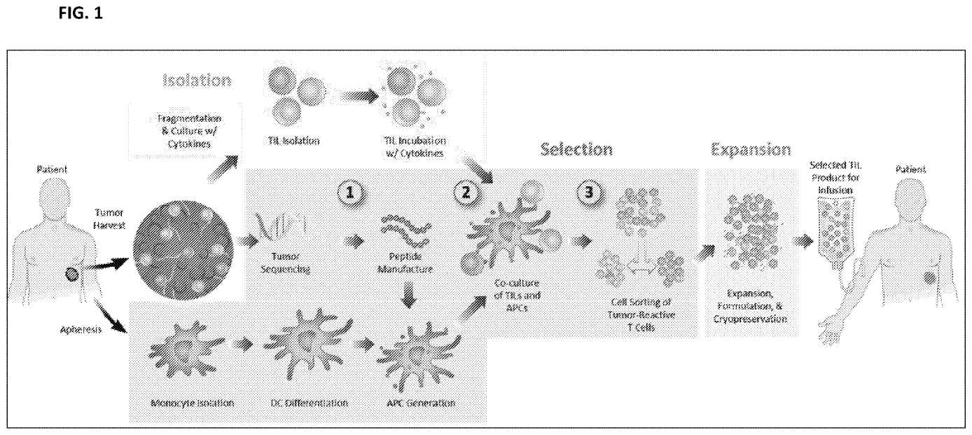

FIG. 1 depicts a schematic of an exemplary process for manufacturing a T cell therapeutic composition in accord with the provided methods. In the exemplary process a tumor sample is obtained from a patient for identification and generation of peptides for use in co-culturing methods with antigen presenting cells (APCs) presenting the peptides and autologous antigen T cells obtained from the same subject. In some cases, a suspension of dissociated tumor cells containing T cells is obtained or provided from the patient, and subjected to an initial pre-expansion with recombinant IL-2 to expand T cells from the tumor, prior to co-culture with antigen presenting cells that have been loaded with a pool of 20mer-40mer peptides identified from the neoantigens and manufactured for presentation on MHC class I and/or MHC class II molecules. Following co-culture, tumor-reactive T cells are selected for cells surface positive for CD134 and/or CD137 by sorting using fluorescence activated cell sorting (FACs), thereby removing potential bystander cells. The selected and sorted cells are then subjected to a rapid expansion protocol with irradiated peripheral blood mononuclear cells (iPBMCs), agonist anti-CD3 antibody (e.g., OKT3), and recombinant IL-2 until a threshold number of cells is obtained, typically 12 to 16 days, such as at or about 14 days. Upon reaching the threshold number of cells, the cells can be harvested and formulated, in some cases concentrated or cryopreserved, and used for administration to a subject such as by infusion. In some aspects, it is contemplated that this enriched TIL product will have superior reactivity as compared to a similar TIL product prepared by a bulk method.

The process can be carried out in the presence of serum-free media containing nutrients. One or more or all of the steps can be carried out in a closed system, such as without exposure of cells to the outside environment.

By virtue of the provided process, the initial small population of tumor reactive T cells expanded from the tumor are enriched for cells that are tumor reactive cells before a subsequent second expansion step, thereby promoting preservation and expansion of cells of interest and limiting expansion of bystander T cells that are not reactive to a tumor antigen and/or that may include cells that exhibit inhibitory activity. The provided methods maximize the number of tumor reactive cells that may be collected by co-culturing all of the cells propagated after the first expansion with peptide-presenting APCs, and then by selecting from among all of the bulk cells after the co-culturing for cells positive for CD134 and/or CD137 before the subsequent second expansion. As demonstrated herein, the provided methods can result in 1000-fold or more expansion of the selected tumor reactive cells with REP, and such cells exhibit robust neoantigen reactivity as demonstrated by cytokine (e.g., IFNγ) production and secretion and production of factors necessary for cytotoxic killing (e.g. CD107a and Granzyme B).

This is in contrast to existing methods that involve passive expansion of bulk T cells in which all T cells from a tumor are subjected to expansion. In such other methods, while total viable cells (TVC) can be greatly expanded by these alternative processes, there is no step of actively ensuring that tumor reactive T cells are predominantly propagated.

In particular aspects, the provided methods can be used for the ex vivo production of a T cell therapy, including for the ex vivo expansion of autologous tumor-reactive T cells. In some embodiments, the methods produce or expand T cells for use in autologous cell therapy for treating cancer. In particular embodiments, the tumor from which the cells are derived is from a colorectal cancer (CRC) and the methods are used to treat CRC in the patient.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. ENRICHED TUMOR REACTIVE TIL COMPOSITIONS

Provided herein are TIL compositions that are enriched for tumor reactive T cells. In some embodiments, the TIL compositions contain primary T cells from a tumor from a subject that have been enriched for tumor reactive T cells by co-culture with autologous APCs presenting neoantigenic peptides from the subject's tumor and expanded ex vivo. In some embodiments, provided TIL compositions can be produced by the provided ex vivo methods for producing TIL compositions.

In some embodiments, the provided TIL composition is a multiclonal population that exhibits TCR diversity and enrichment of T cell receptors (TCRs) reactive to neoantigens (i.e., neoantigenic peptides). In some embodiments, the multiclonal population is an oligoclonal population that exhibits TCR diversity and enrichment of different TCR clonotypes reactive to neoantigens. In some embodiments, the TIL composition contains at least 10 different TCR clonotypes that exhibit neoantigen reactivity. In some embodiments, the TIL composition contains at least 11, 12, 12, 14, 15, 16, 17, 18, 19, 20 or more different TCR clonotypes that exhibit neoantigen reactivity. In some embodiments, the TIL composition contains more than 20 different TCR clonotypes, such as 20 to 100 different TCR clonotypes, such as 20, 30, 40, 50, 60, 70, 80, 90 or 100 different TCR clonotypes or any value between any of the foregoing. In some embodiments, the TIL composition contains 30 to 80 different TCR clonotypes. In some embodiments, the TIL composition contains 40 to 60 different TCR clonotypes.

In some embodiments, the population is enriched in tumor reactive T cells and comprises at least 10 different TCR clonotypes each with a frequency in the population of at least 2.0%. In some embodiments, the population is enriched in tumor reactive T cells and comprises at least 11 different TCR clonotypes each with a frequency in the population of at least 2.0%. In some embodiments, the population is enriched in tumor reactive T cells and comprises at least 12 different TCR clonotypes each with a frequency in the population of at least 2.0%. In some embodiments, 8 to 15 different T cell receptor (TCR) clonotypes make up at least 50% of the TCR frequency in the population. In some embodiments, 9 to 12 different TCR clonotypes make up at least 50% of the TCR frequency in the population.

In some embodiments, the population is enriched in tumor reactive T cells and comprises at least 10 different TCR clonotypes each with a frequency in the population of at least 1.0%. In some embodiments, the population is enriched in tumor reactive T cells and comprises at least 11 different TCR clonotypes each with a frequency in the population of at least 1.0%. In some embodiments, the population is enriched in tumor reactive T cells and comprises at least 12 different TCR clonotypes each with a frequency in the population of at least 1.0%. In some embodiments, 8 to 15 different T cell receptor (TCR) clonotypes make up at least 50% of the TCR frequency in the population. In some embodiments, 9 to 12 different TCR clonotypes make up at least 50% of the TCR frequency in the population.

In some embodiments, the population is enriched in tumor reactive T cells and comprises at least 20 different TCR clonotypes each with a frequency in the population of at least 2.0%. In some embodiments, the population is enriched in tumor reactive T cells and comprises at least 25 different TCR clonotypes each with a frequency in the population of at least 2.0%. In some embodiments, the population is enriched in tumor reactive T cells and comprises at least 30 different TCR clonotypes each with a frequency in the population of at least 2.0%. In some embodiments, the population is enriched in tumor reactive T cells and comprises at least 40 different TCR clonotypes each with a frequency in the population of at least 2.0%. In some embodiments, 8 to 15 different T cell receptor (TCR) clonotypes make up at least 50% of the TCR frequency in the population. In some embodiments, 9 to 12 different TCR clonotypes make up at least 50% of the TCR frequency in the population.

In some embodiments, the top40 TCR clonotypes make up at least 75% of the TCR frequency in the population. In some embodiments, the top40 TCR clonotypes make up at least 80% of the TCR frequency in the population. In some embodiments, the top40 TCR clonotypes make up at least 85% of the TCR frequency in the population. In some embodiments, the top40 TCR clonotypes make up at least 90% of the TCR frequency in the population.

In some embodiments, the neoantigen reactivity of the TCR clonotypes is reactivity to at least one CD4 antigen and at least one CD8 antigen. In some embodiments, the neoantigen reactivity of the TCR clonotypes is for at least 2 peptide antigens, in which at least one peptide antigen is a CD4 antigen and at least one peptide antigen is a CD8 antigen.

In some embodiments, the neoantigen reactivity of the TCR clonotypes is for 2 to 6 different peptide antigens. In some embodiments, the neoantigen reactivity of the TCR clonotypes is for 2 to 4 peptide antigens. In some embodiments, the neoantigen reactivity of the TCR clonotypes is for 2 peptide antigens. In some embodiments, the neoantigen reactivity of the TCR clonotypes is for 2 peptide antigens in which one is a CD8 antigen and one is a CD4 antigen.

In some embodiments, the neoantigen reactivity of the TCR clonotypes is for 2 to 100 different peptide antigens. In some embodiments, the neoantigen reactivity of the TCR clonotypes is for 2 to 80 different peptide antigens. In some embodiments, the neoantigen reactivity of the TCR clonotypes is for 2 to 60 different peptide antigens. In some embodiments, the neoantigen reactivity of the TCR clonotypes is for 2 to 40 different peptide antigens. In some embodiments, the neoantigen reactivity of the TCR clonotypes is for 2 to 20 different peptide antigens. In some embodiments, the neoantigen reactivity of the TCR clonotypes is for 2 to 10 different peptide antigens. In some embodiments, the neoantigen reactivity of the TCR clonotypes is for 2 to 6 different peptide antigens. In some embodiments, the neoantigen reactivity of the TCR clonotypes is for 2 to 4 different peptide antigens.

In some embodiments, the neoantigen reactivity of the TCR clonotypes is for 4 to 100 different peptide antigens. In some embodiments, the neoantigen reactivity of the TCR clonotypes is for 4 to 80 different peptide antigens. In some embodiments, the neoantigen reactivity of the TCR clonotypes is for 4 to 60 different peptide antigens. In some embodiments, the neoantigen reactivity of the TCR clonotypes is for 4 to 40 different peptide antigens. In some embodiments, the neoantigen reactivity of the TCR clonotypes is for 4 to 20 different peptide antigens. In some embodiments, the neoantigen reactivity of the TCR clonotypes is for 4 to 10 different peptide antigens. In some embodiments, the neoantigen reactivity of the TCR clonotypes is for 4 to 6 different peptide antigens.

In some embodiments, the neoantigen reactivity of the TCR clonotypes is for 6 to 100 different peptide antigens. In some embodiments, the neoantigen reactivity of the TCR clonotypes is for 6 to 80 different peptide antigens. In some embodiments, the neoantigen reactivity of the TCR clonotypes is for 6 to 60 different peptide antigens. In some embodiments, the neoantigen reactivity of the TCR clonotypes is for 6 to 40 different peptide antigens. In some embodiments, the neoantigen reactivity of the TCR clonotypes is for 6 to 20 different peptide antigens. In some embodiments, the neoantigen reactivity of the TCR clonotypes is for 6 to 10 different peptide antigens.

In some embodiments, the neoantigen reactivity of the TCR clonotypes is for 2, 4, 6, 8, 10, 20, 30, 40, 50, 60, 70, 80, 90, or 100 different peptide antigens. In some embodiments, the neoantigen reactivity of the TCR clonotypes is for 2 different peptide antigens. In some embodiments, the neoantigen reactivity of the TCR clonotypes is for 4 different peptide antigens. In some embodiments, the neoantigen reactivity of the TCR clonotypes is for 6 different peptide antigens. In some embodiments, the neoantigen reactivity of the TCR clonotypes is for 8 different peptide antigens. In some embodiments, the neoantigen reactivity of the TCR clonotypes is for 10 different peptide antigens. In some embodiments, the neoantigen reactivity of the TCR clonotypes is for 20 different peptide antigens. In some embodiments, the neoantigen reactivity of the TCR clonotypes is for 40 different peptide antigens. In some embodiments, the neoantigen reactivity of the TCR clonotypes is for 60 different peptide antigens. In some embodiments, the neoantigen reactivity of the TCR clonotypes is for 80 different peptide antigens. In some embodiments, the neoantigen reactivity of the TCR clonotypes is for 100 different peptide antigens.

Various methods for assessing the TCR repertoire for clonotype identification and TCR repertoire analysis are known (see e.g., Rosati et al. (2017) BMC Biotechnology, 17:61; Friedensohn et al. (2016) Trends in Biotechnology, 35:203-214). In some aspects, the methods involve high-throughput or next-generation sequencing methods. In some embodiments, the frequency and variety of different clones present in the population or composition can be determined. In some embodiments, the compositions can be assessed the clonality, clonal diversity or clonal heterogeneity of the cells in the population of the composition of cells, for example, based on the determined frequency and/or variety of clonotypes present in the population or composition. In some embodiments, single-cell sequencing methods are carried out to identify a clonotype on a particular cell. In certain aspects, paired αβ TCR sequencing methods are used (see e.g., WO2017053902A1). In some embodiments, sequencing methods are carried out on DNA, such as genomic DNA or complementary DNA. In some embodiments, sequencing methods are carried out on RNA. In some embodiments, high-throughput or next-generation sequencing of TCR sequences or by sequencing the whole genome or transcriptome (e.g., RNAseq). In some aspects, the methods used are RNAseq-based methods.

In some embodiments, T cell clonotype assessment and clonality and diversity in various T cell populations or compositions or samples containing T cells, are determined using high-throughput sequencing of all or a portion of the TCR genes or based on sequences obtained from high-throughput whole genome or transcriptome analysis, on the population or composition of cells, and/or in a single cell. In some embodiments, the provided methods can include various features of the methods as described in WO2016/044227, WO2016/176322, WO2012/048340, WO2012/048341, WO2014/144495, WO2017/053902, WO2017/053903 or WO2017/053905, each incorporated by reference in their entirety.

The clonotypes of a cell or the clonotypes present in a population or composition of cells, in some examples, may be determined by TCR sequencing. In some embodiments, sequencing methods that can be employed include high-throughput or next-generation sequencing as is known in the art. In some aspects, next-generation sequencing methods can be employed, using genomic DNA or cDNA from T cells, to assess the TCR repertoire, including sequences encoding the complementarity-determining region 3 (CDR3 ). In some embodiments, whole transcriptome sequencing by RNAseq can be employed. In some aspects, the TCR repertoire information, e.g., TCR sequences and relative frequency, can be constructed or extracted from whole transcriptome sequencing (e.g., by RNAseq). For example, in some aspects, computational methods such as MIXCR (Such as those described in Bolotin et al. Nature Methods 12(2015 ) 380-381, Bolotin et al., Nature Biotechnology 35 (2017) 908-911) or IMREP (Mangul et al., bioRxiv (2017) 089235) can be utilized to determine the repertoire TCR sequences or a portion thereof (e.g., CDR3) from whole transcriptome RNAseq results. In some embodiments, single-cell sequencing methods can be used. In some embodiments, clonotypes can be assessed or determined by spectratype analysis (a measure of the TCR Vβ, Vα, Vγ, or Vδ chain hypervariable region repertoire). Clonotypes can also be determined by generation and characterization of antigen-specific clones to an antigen of interest.

In some embodiments, T cell clonotype assessment are determined using high-throughput sequencing of all or a portion of the TCR genes or based on sequences obtained from high-throughput whole genome or transcriptome analysis, on the population or composition of cells, and/or in a single cell. In some embodiments, bulk sequencing of targeted sequences (e.g., TCR chains or portion thereof) or bulk whole genome or transcriptome sequencing (e.g., by RNAseq) can be used to determine the clonotypes present in the cells in the population or composition. In some aspects, T cell clonotype assessment can involve sequencing of a portion of the variable region of one or more native TCR chains, such as the complementarity-determining region 3 (CDR3 ). In some aspects, single cell sequencing can be employed. In some embodiments, the provided methods can include various features of the methods as described in WO2016/044227, WO2016/176322, WO2012/048340, WO2012/048341, WO2014/144495, WO2017/053902, WO2017/053903 or WO2017/053905, each incorporated by reference in their entirety. In some embodiments, for target TCR molecules, the genes encoding chains of a TCR can be obtained from genomic DNA or mRNA of immune cells or T cells.

In some embodiments, the composition exhibits clonal diversity, i.e., is multiclonal, such as oligoclonal. In some cases, the clonal diversity is determined based on the relative frequency of the one or more clonotypes and/or one or more TCR sequences. For instance, oligoclonality or grammatical variations thereof refer to clonotypes derived from a few clones. The relative number of specificities to determine if a multiclonal population is oligoclonal is not necessarily a defined number but is generally 10 or less antigen specificities, but can be higher or lower. In some embodiments, an oligoclonal composition has 2-10 different specificities. In some embodiments, the determining the clonal diversity is represented as clonality, Shannon-adjusted clonality or top 25 clonality of each of the plurality of samples. In some embodiments, the determining the clonal diversity is represented as Shannon-adjusted clonality in a composition.

In some embodiments, the cells of the provided TIL compositions exhibit one or more phenotypic or functional markers. In some cases, such cells include cells positive or negative for one or more phenotypic marker or functional feature or attribute.

As used herein, a statement that a cell or population of cells is “positive” for a particular marker, function or attribute refers to the detectable presence on or in the cell of a particular marker, such as a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker, function or attribute refers to the absence of substantial detectable presence on or in the cell of a particular marker, such as a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker. Exemplary markers, functions and attributes of provided TIL compositions are described below. In some embodiments, the TIL composition is characterized by any one or more of such features, such as 2, 3, 4, 5 or more of such features. For instance, a provided TIL composition may be characterized by presence or absence of one or more T cell markers, effector memory phenotype markers, exhaustion markers, the ability to produce or secrete cytokines and/or the ability to produce or secrete a cytotoxic factor, such as described below. Any 2, 3, 4, 5 or more of any of such features may be present in a TIL composition as described.

In some embodiments, a provided TIL composition comprises CD3+ T cells as a percentage of total cells in the population that is greater than or greater than about 85%, such as greater than or greater than about 90%, greater than or greater than about 95%, greater than or greater than about 97% or greater than or greater than about 98%. In some embodiments, a provided TIL composition comprises CD3+ T cells as a percentage of total cells in the population that is greater than or greater than about 90%. In some embodiments, a provided TIL composition comprises CD3+ T cells as a percentage of total cells in the population that is greater than or greater than about 95%. In some embodiments, a provided TIL composition comprises CD3+ T cells as a percentage of total cells in the population that is greater than or greater than about 98%. In some embodiments, the composition contains CD4+ T cells and CD8+ T cells as a percentage of total cells in the population that is greater than or greater than about 85%, greater than or greater than about 90%, greater than or greater than about 95%, greater than or greater than about 97% or greater than or greater than about 98%.

In some embodiments, the composition contains a ratio of CD4+ T cells to CD8+ T cells that is between at or about 5:1 to 50:1, between 5:1 to 25:1, between 5:1 to 20:1, between 5:1 to 15:1, between 5:1 to 10:1, between 10:1 to 50:1, between 10:1 to 25:1, between 10:1 to 20:1, between 10:1 to 15:1, between 15:1 to 50:1, between 15:1 to 25:1, between 15:1 to 20:1, between 20:1 to 50:1, between 20:1 to 25:1 or between 25:1 to 50:1. In some embodiments, the composition contains a ratio of CD4+ T cells to CD8+ T cells that is at or about 10:1 to 25:1. In some embodiments, the composition contains a ration of CD4+ T cells to CD8+ T cells that is about 20:1.

In some embodiments, among CD3+ T cells in the TIL composition, or CD4+ and/or CD8+ T cell subsets thereof, greater than 50% express a marker of an effector memory phenotype. In some embodiments, among CD3+ T cells in the TIL composition, or CD4+ and/or CD8+ T cell subsets thereof, greater than about 50% express an effector memory phenotype marker, greater than about 60% express an effector memory phenotype marker, greater than about 70% express an effector memory phenotype marker, greater than about 80% express an effector memory phenotype marker, or greater than 90% express an effector memory phenotype marker. In some embodiments, the effector memory phenotype is characterized by surface marker expression of one or more of CD45RA−, CD45RO+, CD62L⁻, CCR7−, CD28−and CD27−. In some embodiments, the effector memory phenotype is characterized by surface marker expression CD45RA−and CCR7−. In some embodiments, the effector memory phenotype is characterized by surface marker expression CD45RA−CD45RO+, CD62L⁻, and CCR7⁻. In some embodiments, the effector memory phenotype is characterized by surface marker expression CD45RA−, CD45RO+, CD62L⁻, CCR7⁻, CD28⁻ and CD27.

In some embodiments, among CD3+ T cells in the TIL composition, or CD4+ and/or CD8+ T cell subsets thereof, greater than about 50% are CD45RA− and CCR7, greater than about 60% are CD45RA− and CCR7−, greater than about 70% are CD45RA− and CCR7−, greater than about 80% are CD45RA− and CCR7−, or greater than 90% are CD45RA− and CCR7−.

In some embodiments, among CD3+ T cells in the TIL composition, or CD4+ and/or CD8+ T cell subsets thereof, greater than about 10% express a central memory T cell marker, greater than about 15% express a central memory T cell marker, greater than about 20% express a central memory T cell marker, or greater than 25% express a central memory T cell marker. In some embodiments, the central memory T cell marker is CD45RA−CCR7+. In some embodiments, among CD3+ T cells in the TIL composition, or CD4+ and/or CD8+ T cell subsets thereof, greater than about 10% are CD45RA−CCR7+, greater than about 15% are CD45RA−CCR7+, greater than about 20% are CD45RA−CCR7+, or greater than 25% are CD45RA−CCR7+.

In some embodiments, among CD3+ T cells in the TIL composition, or CD4+ and/or CD8+ T cell subsets thereof, greater than about 10% express a central memory T cell marker (e.g., CD45RA−CCR7+) and greater than about 60% express an effector memory phenotype marker (e.g.

CD45RA−CCR7−). In some embodiments, among CD3+ T cells in the TIL composition, or CD4+ and/or CD8+ T cell subsets thereof, greater than about 15% express a central memory T cell marker (e.g., CD45RA−CCR7+) and greater than about 60% express an effector memory phenotype marker (e.g. CD45RA−CCR7−). In some embodiments, among CD3+ T cells in the TIL composition, or CD4+ and/or CD8+ T cell subsets thereof, greater than about 20% express a central memory T cell marker (e.g., CD45RA−CCR7+) and greater than about 60% express an effector memory phenotype marker (e.g. CD45RA−CCR7−). In some embodiments, among CD3+ T cells in the TIL composition, or CD4+ and/or CD8+ T cell subsets thereof, greater than about 25% express a central memory T cell marker (e.g., CD45RA−CCR7+) and greater than about 60% express an effector memory phenotype marker (e.g. CD45RA−CCR7−).

In some embodiments, among CD3+ T cells in the TIL composition, or CD4+ and/or CD8+ T cell subsets thereof, greater than about 10% express a central memory T cell marker (e.g., CD45RA−CCR7+) and greater than about 70% express an effector memory phenotype marker (e.g.

CD45RA−CCR7−). In some embodiments, among CD3+ T cells in the TIL composition, or CD4+ and/or CD8+ T cell subsets thereof, greater than about 15% express a central memory T cell marker (e.g., CD45RA−CCR7+) and greater than about 70% express an effector memory phenotype marker (e.g. CD45RA−CCR7−). In some embodiments, among CD3+ T cells in the TIL composition, or CD4+ and/or CD8+ T cell subsets thereof, greater than about 20% express a central memory T cell marker (e.g., CD45RA−CCR7+) and greater than about 70% express an effector memory phenotype marker (e.g. CD45RA−CCR7−). In some embodiments, among CD3+ T cells in the TIL composition, or CD4+ and/or CD8+ T cell subsets thereof, greater than about 25% express a central memory T cell marker (e.g., CD45RA−CCR7+) and greater than about 70% express an effector memory phenotype marker (e.g. CD45RA−CCR7−).

In some embodiments, among CD3+ T cells in the TIL composition, or CD4+ and/or CD8+ T cell subsets thereof, greater than about 10% express a central memory T cell marker (e.g., CD45RA−CCR7+) and greater than about 80% express an effector memory phenotype marker (e.g.

CD45RA−CCR7−). In some embodiments, among CD3+ T cells in the TIL composition, or CD4+ and/or CD8+ T cell subsets thereof, greater than about 15% express a central memory T cell marker (e.g., CD45RA−CCR7+) and greater than about 80% express an effector memory phenotype marker (e.g. CD45RA−CCR7−). In some embodiments, among CD3+ T cells in the TIL composition, or CD4+ and/or CD8+ T cell subsets thereof, about 20% express a central memory T cell marker (e.g., CD45RA−CCR7+) and about 80% express an effector memory phenotype marker (e.g. CD45RA−CCR7−).

In some embodiments, among CD3+ T cells in the TIL composition, or CD4+ and/or CD8+ T cell subsets thereof, about 10% express a central memory T cell marker (e.g., CD45RA−CCR7+) and about 90% express an effector memory phenotype marker (e.g. CD45RA−CCR7−).

In some embodiments, among CD3+ T cells in the TIL composition, or CD4+ and/or CD8+ T cell subsets thereof, less than 30% of the cells express an exhaustion phenotype. In some embodiments, among CD3+ T cells in the TIL composition, or CD4+ and/or CD8+ T cell subsets thereof, less than about 25% express an exhaustion phenotype, less than about 20% express an exhaustion phenotype, less than about 15% express an exhaustion phenotype, or less than about 10% express an exhaustion phenotype. In some cases, exhaustion can be assessed by monitoring loss of T cell function, such as reduced or decreased neoantigen-specific reactivity, such as a reduced or decreased ability to produce cytokines or to drive cytolytic activity against target antigen. In some cases, exhaustion also can be assessed by monitoring expression of surface markers on T cells (e.g., CD3+ T cells, or a CD4 and/or CD4 T cell subset thereof) that are associated with an exhaustion phenotype. Among exhaustion markers are inhibitory receptors such as PD-1, CTLA-4, LAG-3 and TIM-3. In some embodiments, the exhaustion phenotype is positive expression for 1, 2, 3 or 4 of such exhaustion markers.

In some embodiments, among CD3+ T cells in the TIL composition, or CD4+ and/or CD8+ T cell subsets thereof, greater than 70% are PD-1⁻. In some embodiments, among CD3+ T cells in the TIL composition, or CD4+ and/or CD8+ T cell subsets thereof, greater than 75% are PD-1⁻, greater than 80% are PD-1⁻, greater than 85% are PD-1⁻, greater than 90% are PD-1⁻, or greater than 95% are PD-1⁻.

In some embodiments, among CD3+ T cells in the TIL composition, or CD4+ and/or CD8+ T cell subsets thereof, greater than 70% are LAG3. In some embodiments, among CD3+ T cells in the TIL composition, or CD4+ and/or CD8+ T cell subsets thereof, greater than 75% are LAG3⁻, greater than 80% are LAG3⁻, greater than 85% are LAG3⁻, greater than 90% are LAG3, or greater than 95% are LAG3⁻.

In some embodiments, among CD3+ T cells in the TIL composition, or CD4+ and/or CD8+ T cell subsets thereof, greater than 70% are PD-1⁻ and LAG3⁻. In some embodiments, among CD3+ T cells in the TIL composition, or CD4+ and/or CD8+ T cell subsets thereof, greater than 75% are PD-1⁻ and LAG3⁻, greater than 80% are PD-1⁻ and LAG3⁻, greater than 85% are PD-1⁻ and LAG3⁻, greater than 90% are PD-1⁻ and LAG3⁻, or greater than 95% are PD-1⁻ and LAG3⁻.

In some embodiments, a provided TIL composition includes about 10-60% tumor-reactive T cells. In some embodiments, a provided TIL composition includes greater than about 15% tumor reactive T cells, greater than about 20% tumor-reactive T cells, greater than about 25% tumor-reactive T cells, greater than about 30% tumor-reactive T cells, greater than about 40% tumor-reactive T cells or greater than about 50% tumor-reactive T cells, or any value between any of the foregoing.

The provided TIL compositions enriched in tumor reactive cells exhibit a number of functional or phenotypic activities that evidence their reactivity to neoantigens (i.e., neoantigenic peptides). In some embodiments, cells can be assessed for any of a number of functional or phenotypic activities, including but not limited to cytotoxic activity, degranulation, ability to produce or secrete cytokines, and expression of one or more intracellular or surface phenotypic markers. Methods to assess such activities are known and are exemplified herein and in working examples.

In some embodiments, upon recognition of neoantigens (i.e., neoantigenic peptides) presented by APC, TILs can become activated. Upon activation, the TILs produce cytokines, chemokines and other factors abundantly and at the same time exhibit potent cytolytic activity. In some embodiments, activation triggers the release of cytoplasmic granules containing granzymes, leading to target cell death. Assays to measure cytokines, chemokines and other soluble factors are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample.

In some embodiments, TILs can be evaluated for phenotype or general functional activity, such as based on IFN-γ and/or granzyme B secretion or other cytokine secretion, in response to a polyclonal stimulation. In some embodiments, the polyclonal stimulation is stimulation of CD3 (e.g., with OKT3). In some embodiments, the in vitro CD3 assay includes OKT3 stimulation. In some embodiments, the in vitro CD3 assay includes washing and seeding TIL into culture plates precoated with OKT3 diluted in phosphate-buffered saline. In some embodiments, the polyclonal stimulation is stimulation of CD3 (e.g., with OKT3) and CD28 to provide a costimulatory signal. In some embodiments, the in vitro assay includes stimulation with an anti-CD3 and anti-CD28 antibody, such as by incubation of cells with Dynabeads. After overnight incubation, the supernatants are harvested and protein in the supernatant is measured by ELISA for cytokines of interest.

In some embodiments, the provided TIL compositions are assessed for tumor or neoantigen reactivity, for example by an in vitro assay. In some embodiments, the assay can be an in vitro autologous tumor assay. In some embodiments, the assay is an in vitro co-culture assay.

In these and related embodiments, the results from such assays (e.g., an in vitro autologous tumor assay or in vitro co-culture assay or like assay) can be used as criteria to characterize the TIL compositions and/or cell populations making up the composition. Such criteria can include without limitation the presence of and/or amounts or levels of one more of the following: cytotoxic activity (e.g., tumor cell killing), cell activation and/or reactivity (e.g., against tumor cells) production and/or secretion of one more of cytokines (e.g., IFN-γ and/or granzyme B secretion) or production or secretion of other compound related to one or more of cytotoxic activity, cell activation, cell reactivity, cell viability or cell exhaustion.

In some embodiments, TILs can be evaluated for cytokine secretion, e.g., IFN-γ and/or granzyme B secretion, in response to co-culture with autologous tumor digest in an in vitro autologous tumor assay. In some embodiments, reference to an in vitro autologous tumor assay is understood to be an assay in which TIL are incubated with non-hematopoietic cells from an autologous primary tumor. In some embodiments, the in vitro autologous tumor assay includes seeding TILs into a culture plate with autologous non-bematopoietic tumor cells (e.g., 1:1 ratio). In some embodiments, the autologous tumor cells are single cell suspensions of CD45 negative (CD45−) cells obtained from a primary tumor. After a period of incubation ranging from 12-24 hours, supernatants are harvested and factor release can be quantified, for example by ELISA.

In some embodiments, TILs can be evaluated for cytokine secretion, e.g., IFN-γ and/or granzyme B secretion, in response to co-culture with APCs loaded with neoantigen (i.e., neoantigenic peptides) in an in vitro co-culture assay. In some embodiments, reference to an in vitro co-culture assay is understood to be an assay in which TIL are incubated with autologous APCs loaded with autologous neoantigenic peptides (hereinafter also referred to as peptide loaded autologous APCs). Io some embodiments, the in vitro co-culture assay includes seeding TILs into a culture plate with autologous irradiated APCs presenting neoantigenic peptide. In some embodiments, the APCs are irradiated. In some embodiments, the APCs are blood-derived APCs, such as B cells or dendritic cells. In some embodiments, the in vitro co-culture assay referenced herein is an assay in which B cells are isolated and expanded from autologous blood or apheresis, such as by culture with CD40L and IL-4 for 14 days before loading with neoantigenic peptide, and then co-culture with TIL at a ratio ranging from 1:1 to 1:5 TIL: APC. After a period of incubation ranging from 12-24 hours, supernatants are harvested and factor release can be quantified, for example by ELISA. In some aspects, an in vitro co-culture assay results in strong T cell activation. Without wishing to be bound by theory, it is considered that the APCs present in the in vitro co-culture assay express robust levels of HLA and costimulatory molecules required to optimally activate T cells, in addition to being pulsed with neoantigenic peptide cognate to the TIL TCR.

In some embodiments, the provided TIL composition includes an increased or greater percentage of cells that exhibit neoantigen reactivity compared to a bulk TIL composition. In particular embodiments, reference to a bulk TIL composition refers to a TIL population expanded from the same input source of tumor cells as the provided TIL composition but that is not enriched for tumor-reactive cells by co-culture with APCs presenting neoantigenic peptides and selection of cells positive for CD134 and CD137. For instance, in some embodiments, a bulk TIL composition refers to a TIL population that is processed the same or substantially the same to the provided TIL composition generated as described in Sections IL. A-E except that is not subject to selection of cells positive for CD134 and CD137 so that the entire bulk population of T cells from the tumor sample are subject to ex vivo expansion. In some embodiments, the neoantigen reactivity is increased by greater than 2-fold, greater than 3-fold, greater than 4-fold, greater than 5-fold, greater than 6-fold, greater than 7-fold, greater than 8-fold, greater than 9-fold, greater than 10-fold, greater than 15-fold, greater than 20-fold, greater than 30-fold, greater than 40-fold, greater than 50-fold or more.

In some embodiments, the provided TIL compositions display higher neoantigen reactivity than a bulk TIL composition in a neoantigen reactivity assay, such as an in vitro co-culture assay or an in vitro autologous tumor assay. In some embodiments, among CD8+ T cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, among CD4+ T cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, among total T cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, among total cells in a provided TIL composition, greater than at or about 15%, greater than at or about 20%, greater than at or about 30%, greater than at or about 40% or greater than at or about 50% exhibit neoantigen reactivity in an in vitro autologous tumor assay. In some embodiments, the TIL composition exhibits greater than about 2-fold more neoantigen reactivity, 3-fold more neoantigen reactivity, 4-fold more neoantigen reactivity, or 5-fold more neoantigen reactivity in an in vitro autologous tumor assay, compared to a bulk TIL composition.

In some embodiments, among CD8+ T cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, among CD4+ T cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, among total T cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, among total cells in a provided TIL composition, greater than at or about 15%, greater than at or about 20%, greater than at or about 30%, greater than at or about 40% or greater than at or about 50% exhibit neoantigen reactivity in an in vitro co-culture assay, e.g., following culture with autologous APCs (e.g. DCs or B cells) presenting neoantigen peptides. In some embodiments, the TIL composition exhibits greater than about 2-fold more neoantigen reactivity, 3-fold more neoantigen reactivity, 4-fold more neoantigen reactivity, or 5-fold more neoantigen reactivity in an in vitro co-culture assay, e.g., following culture with autologous APCs (e.g., DCs or B cells) presenting neoantigen peptides, compared to a bulk TIL composition.

In some embodiments, TIL composition can include an increased or greater percentages of CD3+ T cells positive for CD134 and CD137 compared to the percentage of such CD3+ T cells positive for CD134 and CD137 present in a bulk TIL population, in a co-culture assay, e.g. following culture with autologous APCs (e.g. DCs or B cells) presenting neoantigenic peptides. In some embodiments, the percentage is increased at least or at least about 1.5-fold, 2-fold, 3-fold, 4-fold, 5-fold, 10-fold, 20-fold or more.

In some embodiments, among cells in the provided TIL composition, greater than at or about 48% are positive for CD134 and CD137, such as greater than at or about 50%, 55%, 60%, 65%, 70%, 75%, 80%, 85% or more are positive for CD134 and CD137. In some embodiments, among cells in the provided TIL composition, greater than at or about 50% are positive for CD134 and CD137. In some embodiments, among cells in the provided TIL composition, greater than at or about 60% are positive for CD134 and CD137. In some embodiments, among cells in the provided TIL composition, greater than at or about 70% are positive for CD134 and CD137. In some embodiments, among cells in the provided TIL composition, greater than at or about 80% are positive for CD134 and CD137.

In some embodiments, the provided TIL compositions display higher effector cytokine responses in a neoantigen reactivity assay, such as an in vitro co-culture assay or an in vitro autologous tumor assay, than a bulk TIL composition. In some embodiments, among CD8+ T cells in a provided TIL composition, greater than at or about 15%, greater than at or about 20%, greater than at or about 30%, greater than at or about 40% or greater than at or about 50% produce IFN-γ in an in vitro co-culture assay, e.g., following culture with autologous APCs (e.g. DCs or B cells) presenting neoantigen peptides. In some embodiments, among CD4+ T cells in a provided TIL composition, greater than at or about 15%, greater than at or about 20%, greater than at or about 30%, greater than at or about 40% or greater than at or about 50% produce IFN-γ in an in vitro co-culture assay, e.g., following culture with autologous APCs (e.g. DCs or B cells) presenting neoantigen peptides. In some embodiments, among total T cells in a provided TIL composition, greater than at or about 15%, greater than at or about 20%, greater than at or about 30%, greater than at or about 40% or greater than at or about 50% produce IFN-γ in an in vitro co-culture assay, e.g., following culture with autologous APCs (e.g. DCs or B cells) presenting neoantigen peptides. In some embodiments, among total cells in a provided TIL composition, greater than at or about 15%, greater than at or about 20%, greater than at or about 30%, greater than at or about 40% or greater than at or about 50% produce IFN-γ in an in vitro co-culture assay, e.g., following culture with autologous APCs (e.g. DCs or B cells) presenting neoantigen peptides.

In some embodiments, among CD8+ T cells in a provided TIL composition, greater than at or about 15%, greater than at or about 20%, greater than at or about 30%, greater than at or about 40% or greater than at or about 50% produce IFN-γ in an in vitro autologous tumor assay. In some embodiments, among CD4+ T cells in a provided TIL composition, greater than at or about 15%, greater than at or about 20%, greater than at or about 30%, greater than at or about 40% or greater than at or about 50% produce IFN-γ in an in vitro autologous tumor assay. In some embodiments, among total T cells in a provided TIL composition, greater than at or about 15%, greater than at or about 20%, greater than at or about 30%, greater than at or about 40% or greater than at or about 50% produce IFN-γ in an in vitro autologous tumor assay. In some embodiments, among total cells in a provided TIL composition, greater than at or about 15%, greater than at or about 20%, greater than at or about 30%, greater than at or about 40% or greater than at or about 50% produce IFN-γ in an in vitro autologous tumor assay.

In some embodiments, the TIL composition produces greater than about 2-fold more IFN-γ following an in vitro co-culture assay, e.g., culture with autologous APCs (e.g., DCs or B cells) presenting neoantigen peptides, compared to a bulk TIL composition. In some embodiments, the TIL composition produces greater than about 3-fold more IFN-γ, 4-fold more IFN-γ, 5-fold more IFN-γ, 6-fold more IFN-γ, 7-fold more IFN-γ, 8-fold more IFN-γ, 9-fold more IFN-γ, 10-fold more IFN-γ or 15-fold more IFN-γ in an in vitro co-culture assay, e.g., following culture with autologous APCs (e.g. DCs or B cells) presenting neoantigen peptides, compared to a bulk TIL composition. In some embodiments, the TIL composition produces greater than about 20-fold more IFN-γ following an in vitro co-culture assay, e.g., culture with autologous APCs (e.g., DCs or B cells) presenting neoantigen peptides, compared to a bulk TIL composition. In some embodiments, the TIL composition produces greater than about 30-fold more IFN-γ, 40-fold more IFN-γ, 50-fold more IFN-γ, 60-fold more IFN-7, 70-fold more IFN-γ, 80-fold more IFN-γ, 90-fold more IFN-γ or 100-fold more IFN-γ in an in vitro co-culture assay, e.g., following culture with autologous APCs (e.g. DCs or B cells) presenting neoantigen peptides, compared to a bulk TIL composition. In some embodiments, the TIL composition produces greater than about 300-fold more IFN-γ, 400-fold more IFN-γ, 500-fold more IFN-γ, 600-fold more IFN-γ, 700-fold more IFN-γ, 800-fold more IFN-γ, 900-fold more IFN-γ or 1000-fold more IFN-γ in an in vitro co-culture assay, e.g., following culture with autologous APCs (e.g. DCs or B cells) presenting neoantigen peptides, compared to a bulk TIL composition.

In some embodiments, the TIL composition produces greater than about 2-fold more IFN-γ following an in vitro autologous tumor assay compared to a bulk TIL composition. In some embodiments, the TIL composition produces greater than about 3-fold more IFN-γ, 4-fold more IFN-γ, 5-fold more IFN-γ, 6-fold more IFN-γ, 7-fold more IFN-γ, 8-fold more IFN-γ, 9-fold more IFN-γ, 10-fold more IFN-γ or 15-fold more IFN-γ in an in vitro co-culture assay, e.g., following culture with autologous APCs (e.g. DCs or B cells) presenting neoantigen peptides, compared to a bulk TIL composition. In some embodiments, the TIL composition produces greater than about 20-fold more IFN-γ following an in vitro autologous tumor assay compared to a bulk TIL composition. In some embodiments, the TIL composition produces greater than about 30-fold more IFN-γ, 40-fold more IFN-γ, 50-fold more IFN-γ, 60-fold more IFNγ, 70-fold more IFN-γ, 80-fold more IFN-γ, 90-fold more IFN-γ or 100-fold more IFN-γ in an in vitro autologous tumor assay compared to a bulk TIL composition. In some embodiments, the TIL composition produces greater than about 300-fold more IFN-γ, 400-fold more IFN-γ, 500-fold more IFN-γ, 600-fold more IFN-γ, 700-fold more IFN-γ, 800-fold more IFN-γ, 900-fold more IFN-γ or 1000-fold more IFN-γ in an in vitro autologous tumor assay compared to a bulk TIL composition.

In some embodiments, the TIL composition produces IFN-γ following an in vitro co-culture assay, e.g., culture with autologous APCs (e.g., DCs or B cells) presenting neoantigen peptides. In some embodiments, the TIL composition produces between 100-500 pg/mL, between 500-1000 pg/mL, between 1,000-2,000 pg/mL, or between 2,000-2,500 pg/mL IFN-γ. In some embodiments, the TIL composition produces between 2,000-4,000 pg/mL, between 2,000-6,000 pg/mL, between 2,000-20,000 pg/mL, between 2,000-30,000 pg/mL, or between 2,000-40,000 pg/mL IFN-γ. In some embodiments, the TIL composition produces between 2,000-2,500 pg/mL, between 2,000-3,000 pg/mL, between 2,000-3,500 pg/mL, between 2,000-4,000 pg/mL, or between 2,000-4,500 pg/mL IFN-γ. In some embodiments, the TIL composition produces between 2,000-1,500,000 pg/mL, between 2,000-1,000,000 pg/mL, between 2,000-500,000 pg/mL, or between 2,000-250,000 pg/mL IFN-γ. In some embodiments, the TIL composition produces between 5,000-1,500,000 pg/mL, between 5,000-1,000,000 pg/mL, between 5,000-500,000 pg/mL, or between 5,000-250,000 pg/mL IFN-γ. In some embodiments, the TIL composition produces between 5,000-1,500,000 pg/mL, between 100,000-1,000,000 pg/mL, between 100,000-500,000 pg/mL, or between 100,000-250,000 pg/mL IFN-γ. In some embodiments, the TIL composition produces 500 pg/mL, 1,000 pg/mL, 1,500 pg/mL, 2,000 pg/mL, 2,500 pg/mL, or 3,000 pg/mL IFN-γ. In some embodiments, the TIL composition produces 2,000 pg/mL, 4,000 pg/mL, 6,000 pg/mL, 8,000 pg/mL, 10,000 pg/mL, or 20,000 pg/mL IFN-γ. In some embodiments, the TIL composition produces 5,000 pg/mL, 10,000 pg/mL, 20,000 pg/mL, 30,000 pg/mL, 40,000 pg/mL, or 50,000 pg/mL IFN-γ. In some embodiments, the TIL composition produces 50,000 pg/mL, 100,000 pg/mL, 200,000 pg/mL, 300,000 pg/mL, 400,000 pg/mL, 500,000 pg/mL, 600,000 pg/mL, 700,000 pg/mL, 800,000 pg/mL, 900,000 pg/mL, 1,000,000 pg/mL, or 1,500,000 pg/mL IFN-γ. In some embodiments, the TIL composition produces 100 pg/mL IFN-γ. In some embodiments, the TIL composition produces 500 pg/mL. IFN-γ. In some embodiments, the TIL composition produces 1,000 pg/mL IFN-γ. In some embodiments, the TIL composition produces 2,000 pg/mL IFN-γ. In some embodiments, the TIL composition produces 4,000 pg/mL. IFN-γ. In some embodiments, the TIL composition produces 5,000 pg/mL IFN-γ. In some embodiments, the TIL composition produces 6,000 pg/mL IFN-γ. In some embodiments, the TIL composition produces 20,000 pg/mL IFN-γ. In some embodiments, the TIL composition produces 30,000 pg/mL IFN-γ. In some embodiments, the TIL composition produces 40,000 pg/mL IFN-γ. In some embodiments, the TIL composition produces 50,000 pg/mL IFN-γ. In some embodiments, the TIL composition produces 500,000 pg/mL IFN-γ. In some embodiments, the TIL composition produces 600,000 pg/mL IFN-γ. In some embodiments, the TIL composition produces 700,000 pg/mL IFN-γ. In some embodiments, the TIL composition produces 800,000 pg/mL IFN-γ. In some embodiments, the TIL composition produces 900,000 pg/mL IFN-γ. In some embodiments, the TIL composition produces 1,000,000 pg/mL IFN-γ. In some embodiments, the TIL composition produces 1,500,000 pg/mL IFN-γ.

In some embodiments, the TIL composition produces IFN-γ following in an in vitro autologous tumor assay. In some embodiments, the TIL composition produces between 100-500 pg/mL, between 500-,1000 pg/mL, between 1,000-2,000 pg/mL, or between 2,000-2,500 pg/mL IFN-γ. In some embodiments, the TIL composition produces between 2,000-4,000 pg/mL, between 2,000-6,000 pg/mL, between 2,000-20,000 pg/mL, between 2,000-30,000 pg/mL, or between 2,000-40,000 pg/mL IFN-γ. In some embodiments, the TIL composition produces between 2,000-2,500 pg/mL, between 2,000-3,000 pg/L, between 2,000-3,500 pg/mL, between 2,000-4,000 pg/mL, or between 2,000-4,500 pg/mL IFN-γ. In some embodiments, the TIL composition produces between 2,000-15,000 pg/mL, between 2,000-10,000 pg/mL, or between 2,000-5000 pg/mL IFN-γ. In some embodiments, the TIL composition produces between 3,000-15,000 pg/mL, between 3,000-10,000 pg/mL, or between 3,000-5000 pg/mL IFN-γ. In some embodiments, the TIL composition produces between 5,000 -15,000 pg/mL, between 5,000-10,000 pg/mL, or between 5,000-5000 pg/mL IFN-γ. In some embodiments, the TIL composition produces 2,000 pg/mL, 5,000 pg/mL, 10,000 pg/mL, or 15,000 pg/mL. IFN-γ. In some embodiments, the TIL composition produces 100 pg/mL. IFN-γ. In some embodiments, the TIL composition produces 500 pg/mL IFN-γ. In some embodiments, the TIL composition produces 1,000 pg/mL IFN-γ. In some embodiments, the TIL composition produces 2,000 pg/mL IFN-γ. In some embodiments, the TIL composition produces 4,000 pg/mL IFN-γ. In some embodiments, the TIL composition produces 5,000 pg/mL IFN-γ. In some embodiments, the TIL composition produces 6,000 pg/mL IFN-γ. In some embodiments, the TIL composition produces 10,000 pg/mL IFN-γ. In some embodiments, the TIL composition produces 15,000 pg/mL IFN-γ. In some embodiments, the TIL composition produces 20,000 pg/mL IFN-γ. In some embodiments, the TIL composition produces 30,000 pg/mL IFN-γ. In some embodiments, the TIL composition produces 40,000 pg/mL IFN-γ.

In some embodiments, among CD8+ T cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides. In some embodiments, among CD4+ T cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides. In some embodiments, among total T cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides. In some embodiments, among total cells in a provided TIL composition, greater than at or about 15%, greater than at or about 20%, greater than at or about 30%, greater than at or about 40% or greater than at or about 50% produce TNF-α in an in vitro co-culture assay e.g., following culture with autologous APCs (e.g. DCs or B cells) presenting neoantigen peptides.

In some embodiments, among CD8+ T cells in a provided TIL composition, greater than at or about 15%, greater than at or about 20%, greater than at or about 30%, greater than at or about 40% or greater than at or about 50% produce TNF-α in an in vitro autologous tumor assay. In some embodiments, among CD4+ T cells in a provided TIL composition, greater than at or about 15%, greater than at or about 20%, greater than at or about 30%, greater than at or about 40% or greater than at or about 50% produce TNF-α in an in vitro autologous tumor assay. In some embodiments, among total T cells in a provided TIL composition, greater than at or about 15%, greater than at or about 20%, greater than at or about 30%, greater than at or about 40% or greater than at or about 50% produce TNF-□ in an in vitro autologous tumor assay. In some embodiments, among total cells in a provided TIL composition, greater than at or about 15%, greater than at or about 20%, greater than at or about 30%, greater than at or about 40% or greater than at or about 50% produce TNF-α in an in vitro autologous tumor assay.

In some embodiments, the TIL composition produces greater than about 50-fold more TNF-α following an in vitro co-culture assay, e.g., culture with autologous APCs (e.g. DCs or B cells) presenting neoantigen peptides, compared to a bulk TIL composition. In some embodiments, the TIL composition produces greater than about 2-fold more, about 5-fold more TNF-c, 10-fold more TNF-α, 15-fold more TNF-α, 20-fold more TNF-α, 25-fold more TNF-α, 30-fold more TNF-α, 35-fold more TNF-α or 40-fold more TNF-α in an in vitro co-culture assay e.g., following culture with autologous APCs (e.g. DCs or B cells) presenting neoantigen peptides, compared to a bulk TIL composition. In some embodiments, the TIL composition produces greater than about 75-fold more TNF-α, 100-fold more TNF-α, 150-fold more TNF-α, 200-fold more TNF-α, 250-fold more TNF-α, 300-fold more TNF-α, 350-fold more TNF-α or 400-fold more TNF-α in an in vitro co-culture assay e.g., following culture with autologous APCs (e.g. DCs or B cells) presenting neoantigen peptides, compared to a bulk TIL composition.

In some embodiments, the TIL composition produces greater than about 50-fold more TNF-α following an in vitro autologous tumor assay compared to a bulk TIL composition. In some embodiments, the TIL composition produces greater than about 2-fold more, about 5-fold more TNF. a, 10-fold more TNF-α, 15-fold more TNF-α, 20-fold more TNF-α, 25-fold more TNF-α, 30-fold more TNF-α, 35-fold more TNF-α or 40-fold more TNF-α in an in vitro autologous tumor assay compared to a bulk TIL composition. In some embodiments, the TIL composition produces greater than about 75-fold more TNF-α, 100-fold more TNF-α, 150-fold more TNF-α, 200-fold more TNF-α, 250-fold more TNF-α, 300-fold more TNF-α, 350-fold more TNF-α or 400-fold more TNF-α in an in vitro autologous tumor assay compared to a bulk TIL composition.

In some embodiments, the TIL composition produces TNF-α in an in vitro co-culture assay, e.g., culture with autologous APCs (e.g., DCs or B cells) presenting neoantigen peptides. In some embodiments, the TIL composition produces between 50-100 pg/mL, between 50 -150 pg/mL, between 50-200 pg/mL, between 50-400 pg/mL, between 50-500 pg/mL, between 50-600 pg/mL, between 50-700 pg/mL, between 50-800 pg/mL, between 50-900 pg/mL, or between 50-1000 pg/mL TNF-α. In some embodiments, the TIL composition produces between 250-2500 pg/mL, between 250-2000 pg/mL, between 250-1500 pg/mL, between 250-1000 pg/mL, between 250-500 pg/mL TNF-α. In some embodiments, the TIL composition produces 250 pg/mL, 500 pg/mL, 1000 pg/mL, 1500 pg/mL, 2000 pg/mL, or 2500 pg/mL, TNF-α. In some embodiments, the TIL composition produces 50 pg/mL TNF-α. In some embodiments, the TIL composition produces 100 pg/mL TNF-α. In some embodiments, the TIL composition produces 200 pg/mL TNF-α. In some embodiments, the TIL composition produces 250 pg/mL TNF-α. In some embodiments, the TIL composition produces 300 pg/mL TNF-α. In some embodiments, the TIL composition produces 350 pg/mL TNF-α. Io some embodiments, the TIL composition produces 400 pg/mL TNF-α. In some embodiments, the TIL composition produces 450 pg/mL TNF-α . In some embodiments, the TIL composition produces 500 pg/mL TNF-α. In some embodiments, the TIL composition produces 600 pg/mL TNF-α. In some embodiments, the TIL composition produces 700 pg/mL TNF-α. In some embodiments, the TIL composition produces 800 pg/mL TNF-α. In some embodiments, the TIL composition produces 900 pg/mL TNF-u . In some embodiments, the TIL composition produces 1000 pg/mL TNF-α. In some embodiments, the TIL composition produces 1500 pg/mL TNF-α. In some embodiments, the TIL composition produces 2000 pg/mL TNF-α. In some embodiments, the TIL composition produces 2500 pg/mL TNF-α.

In some embodiments, the TIL composition produces TNF-α following an in vitro autologous tumor assay. In some embodiments, the TIL composition produces between 50-100 pg/mL, between 50 -150 pg/mL, between 50-200 pg/mL, between 50-400 pg/mL, between 50-500 pg/mL, between 50-600 pg/mL, between 50-700 pg/mL, between 50-800 pg/mL, between 50-900 pg/mL, or between 50-1000 pg/mL TNF-α. In some embodiments, the TIL composition produces between 250-2500 pg/mL, between 250-2000 pg/mL, between 250-1500 pg/mL, between 250-1000 pg/mL, between 250-500 pg/mL TNF-α. In some embodiments, the TIL composition produces 250 pg/mL, 500 pg/mL, 1000 pg/mL, 1500 pg/mL, 2000 pg/mL, or 2500 pg/mL, TNF-α. In some embodiments, the TIL composition produces 50 pg/mL TNF-α. In some embodiments, the TIL composition produces 100 pg/mL TNF-α. In some embodiments, the TIL composition produces 200 pg/mL TNF-α. In some embodiments, the TIL composition produces 250 pg/mL TNF-α. In some embodiments, the TIL composition produces 300 pg/mL TNF-α. In some embodiments, the TIL composition produces 350 pg/mL TNF-α. In some embodiments, the TIL composition produces 400 pg/mL TNF-α. In some embodiments, the TIL composition produces 450 pg/mL TNF-α. In some embodiments, the TIL composition produces 500 pg/mL TNF-α. In some embodiments, the TIL composition produces 600 pg/mL TNF-α. In some embodiments, the TIL composition produces 700 pg/mL TNF-α. In some embodiments, the TIL composition produces 800 pg/mL TNF-α. In some embodiments, the TIL composition produces 900 pg/mL TNF-α . In some embodiments, the TIL composition produces 1000 pg/mL TNF-α. In some embodiments, the TIL composition produces 1500 pg/mL TNF-α. In some embodiments, the TIL composition produces 2000 pg/mL TNF-α. In some embodiments, the TIL composition produces 2500 pg/ml TNF-α.

In some embodiments, the cytotoxic activity can be determined based on the ability to produce or secrete granzyme B in a neoantigen reactivity assays, such as an in vitro co-culture assay or an in vitro autologous tumor assay. In some embodiments, the TIL composition produces greater than about 10-fold more granzyme B in an in vitro co-culture assay, e.g., following culture with autologous APCs (e.g., DCs or B cells) presenting neoantigen peptides, compared to bulk TIL composition. In some embodiments, the TIL composition produces greater than about 20-fold more granzyme B, 30-fold more granzyme B, 40-fold more granzyme B or 50-fold more granzyme B in an in vitro co-culture assay, e.g., following culture with autologous APCs (e.g., DCs or B cells) presenting neoantigen peptides, compared to a bulk TIL composition. In some embodiments, the TIL composition produces greater than about 100-fold more granzyme B in an in vitro co-culture assay, e.g., following culture with autologous APCs (e.g., DCs or B cells) presenting neoantigen peptides, compared to a bulk TIL composition. In some embodiments, the TIL composition produces greater than about 200-fold more granzyme B, 300-fold more granzyme B, 400-fold more granzyme B or 500-fold more granzyme B in an in vitro co-culture assay, e.g., following culture with autologous APCs (e.g., DCs or B cells) presenting neoantigen peptides, compared to a bulk TIL composition. In some embodiments, the TIL composition produces greater than about 1000-fold more granzyme B in an in vitro co-culture assay, e.g., following culture with autologous APCs (e.g., DCs or B cells) presenting neoantigen peptides, compared to a bulk TIL composition. In some embodiments, the TIL composition produces greater than about 2000-fold more granzyme B, 3000-fold more granzyme B, 4000-fold more granzyme B or 5000-fold more granzyme B in an in vitro co-culture assay, e.g., following culture with autologous APCs (e.g., DCs or B cells) presenting neoantigen peptides, compared to a bulk TIL composition. In some embodiments, the TIL composition produces greater than about 10,000-fold more granzyme B in an in vitro co-culture assay, e.g., following culture with autologous APCs (e.g. DCs or B cells) presenting neoantigen peptides, compared to a bulk TIL composition.

In some embodiments, the TIL composition produces greater than about 10-fold more granzyme B in an in vitro autologous tumor assay compared to a bulk TIL composition. In some embodiments, the TIL composition produces greater than about 20-fold more granzyme B, 30-fold more granzyme B, 40-fold more granzyme B or 50-fold more granzyme B in an in vitro autologous tumor assay compared to a bulk TIL composition. In some embodiments, the TIL composition produces greater than about 100-fold more granzyme B in an in vitro autologous tumor assay compared to a bulk TIL composition. In some embodiments, the TIL composition produces greater than about 200-fold more granzyme B, 300-fold more granzyme B, 400-fold more granzyme B or 500-fold more granzyme B in an in vitro autologous tumor assay compared to a bulk TIL composition. In some embodiments, the TIL composition produces greater than about 1000-fold more granzyme B in an in vitro autologous tumor assay compared to a bulk TIL composition. In some embodiments, the TIL composition produces greater than about 2000-fold more granzyme B, 3000-fold more granzyme B, 4000-fold more granzyme B or 5000-fold more granzyme B in an in vitro autologous tumor assay compared to a bulk TIL composition. In some embodiments, the TIL composition produces greater than about 10,000-fold more granzyme B in an in vitro autologous tumor assay compared to a bulk TIL composition.

In some embodiments, the TIL composition produces granzyme B in an in vitro co-culture assay, e.g., culture with autologous APCs (e.g. DCs or B cells) presenting neoantigen peptides. In some embodiments, the TIL composition produces between 5,000-10,000 pg/mL, between 5,000 -15,000 pg/mL, between 5,000-20,000 pg/mL, between 5,000 -25,000 pg/mL, between 5,000-30,000 pg/mL granzyme B, between 5,000 -75,000 pg/mL granzyme B. In some embodiments, the TIL composition produces between 50,000-600,000 pg/mL, between 50,000-500,000 pg/mL, between 50,000-400,000 pg/mL, between 50,000-300,00 pg/mL, between 200,00-500 pg/mL granzyme B, between 200,000-500 pg/mL granzyme B. In some embodiments, the TIL composition produces 5,000 pg/mL, 10,000 pg/mL, 20,000 pg/mL, 25,000 pg/mL, 50,000 pg/mL, 75,000 pg/mL or 100,000 pg/mL, granzyme B. In some embodiments, the TIL composition produces 50,000 pg/mL, 100,000 pg/mL, 200,000 pg/mL, 300,000 pg/mL, 400,000 pg/mL, 500,000 pg/mL or 600,000 pg/mL, granzyme B. In some embodiments, the TIL composition produces 5,000 pg/mL granzyme B. In some embodiments, the TIL composition produces 10,000 pg/mL granzyme B. In some embodiments, the TIL composition produces 20,000 pg/mL granzyme B. In some embodiments, the TIL composition produces 25,000 pg/mL granzyme B. In some embodiments, the TIL composition produces 50,000 pg/mL granzyme B. In some embodiments, the TIL composition produces 55,000 pg/mL granzyme B. In some embodiments, the TIL composition produces 75,000 pg/mL granzyme B. In some embodiments, the TIL composition produces 100,000 pg/mL granzyme B. In some embodiments, the TIL composition produces 200,000 pg/mL granzyme B. In some embodiments, the TIL composition produces 300,000 pg/mL granzyme B. In some embodiments, the TIL composition produces 400,000 pg/mL granzyme B. In some embodiments, the TIL composition produces 500,000 pg/mL granzyme B. In some embodiments, the TIL composition produces 600,000 pg/mL granzyme B.

In some embodiments, the TIL composition produces granzyme B in an autologous tumor assay. In some embodiments, the TIL composition produces between 200-3,000 pg/mL, between 200-1,000 pg/mL, or between 200-500 pg/mL granzyme B. In some embodiments, the TIL composition produces between 300-3,000 pg/mL, between 300-1,000 pg/mL, or between 300-500 pg/mL granzyme B. In some embodiments, the TIL composition produces between 5,000-10,000 pg/mL, between 5,000 -15,000 pg/mL, between 5,000-20,000 pg/mL, between 5,000 -25,000 pg/mL, between 5,000-30,000 pg/mL granzyme B, between 5,000 -75,000 pg/mL granzyme B. In some embodiments, the TIL composition produces 200 pg/mL, 500 pg/mL, 1,000 pg/mL, or 3,000 pg/mL granzyme B. In some embodiments, the TIL composition produces 1,000 pg/mL granzyme B. In some embodiments, the TIL composition produces 3,000 pg/mL granzyme B. In some embodiments, the TIL composition produces 5,000 pg/mL granzyme B. In some embodiments, the TIL composition produces 10,000 pg/mL granzyme B. In some embodiments, the TIL composition produces 20,000 pg/mL granzyme B. In some embodiments, the TIL composition produces 25,000 pg/mL granzyme B. In some embodiments, the TIL composition produces 50,000 pg/mL granzyme B. In some embodiments, the TIL composition produces 55,000 pg/mL granzyme B. In some embodiments, the TIL composition produces 75,000 pg/mL granzyme B. In some embodiments, the TIL composition produces 100,000 pg/mL granzyme B.

In some embodiments, the provided TIL compositions display higher degranulation responses in a neoantigen reactivity assay, such as in in vitro co-culture assay or an in vitro autologous tumor assay, than a bulk TIL composition. In some embodiments, among CD8+ T cells in a provided TIL composition, greater than at or about 15%, greater than at or about 20%, greater than at or about 30%, greater than at or about 40% or greater than at or about 50% exhibit degranulation in an in vitro co-culture assay e.g., following culture with autologous APCs (e.g. DCs or B cells) presenting neoantigen peptides. In some embodiments, degranulation activity can be measured by CD107a expression. In some embodiments, among CD8+ T cells in a provided TIL composition, greater than at or about 15%, greater than at or about 20%, greater than at or about 30%, greater than at or about 40% or greater than at or about 50% express CD107a in an in vitro co-culture assay, e.g., following culture with autologous APCs (e.g. DCs or B cells) presenting neoantigen peptides. In some embodiments, among CD8+ T cells in a provided TIL composition, greater than at or about 10% express CD107a in an in vitro co-culture assay, e.g., following culture with autologous APCs (e.g. DCs or B cells) presenting neoantigen peptides. In some embodiments, among CD8+ T cells in a provided TIL composition, greater than at or about 20% express CD107a in an in vitro co-culture assay, e.g., following culture with autologous APCs (e.g. DCs or B cells) presenting neoantigen peptides. In some embodiments, among CD8+ T cells in a provided TIL composition, greater than at or about 25% express CD107a in an in vitro co-culture assay, e.g., following culture with autologous APCs (e.g. DCs or B cells) presenting neoantigen peptides.

In some embodiments, among CD4+ T cells in a provided TIL composition, greater than at or about 15%, greater than at or about 20%, greater than at or about 30%, greater than at or about 40% or greater than at or about 50% exhibit degranulation in an in vitro co-culture assay, e.g., following culture with autologous APCs (e.g. DCs or B cells) presenting neoantigen peptides. In some embodiments, degranulation activity can be measured by CD107a expression. In some embodiments, among CD4+ T cells in a provided TIL composition, greater than at or about 15%, greater than at or about 20%, greater than at or about 30%, greater than at or about 40% or greater than at or about 50% express CD107a in an in vitro co-culture assay, e.g., following culture with autologous APCs (e.g. DCs or B cells) presenting neoantigen peptides. In some embodiments, among CD4+ T cells in a provided TIL composition, greater than at or about 5% express CD107a in an in vitro co-culture assay, e.g., following culture with autologous APCs (e.g. DCs or B cells) presenting neoantigen peptides. In some embodiments, among CD4+ T cells in a provided TIL composition, greater than at or about 10% express CD107a in an in vitro co-culture assay e.g., following culture with autologous APCs (e.g. DCs or B cells) presenting neoantigen peptides. In some embodiments, among CD8+ T cells in a provided TIL composition, greater than at or about 15% express CD107a in an in vitro co-culture assay, e.g., following culture with autologous APCs (e.g. DCs or B cells) presenting neoantigen peptides.

In some embodiments, among CD8+ T cells in a provided TIL composition, greater than at or about 15%, greater than at or about 20%, greater than at or about 30%, greater than at or about 40% or greater than at or about 50% exhibit degranulation in an in vitro autologous tumor assay. In some embodiments, degranulation activity can be measured by CD107a expression. In some embodiments, among CD8+ T cells in a provided TIL composition, greater than at or about 15%, greater than at or about 20%, greater than at or about 30%, greater than at or about 40% or greater than at or about 50% express CD107a in an in vitro autologous tumor assay. In some embodiments, among CD8+ T cells in a provided TIL composition, greater than at or about 10% express CD107a in an in vitro autologous tumor assay. In some embodiments, among CD8+ T cells in a provided TIL composition, greater than at or about 20% express CD107a in an in vitro autologous tumor assay. In some embodiments, among CD8+ T cells in a provided TIL composition, greater than at or about 25% express CD107a in an in vitro autologous tumor assay.

In some embodiments, among CD4+ T cells in a provided TIL composition, greater than at or about 15%, greater than at or about 20%, greater than at or about 30%, greater than at or about 40% or greater than at or about 50% exhibit degranulation in an in vitro autologous tumor assay. In some embodiments, degranulation activity can be measured by CD107a expression. In some embodiments, among CD4+ T cells in a provided TIL composition, greater than at or about 15%, greater than at or about 20%, greater than at or about 30%, greater than at or about 40% or greater than at or about 50% express CD107a in an in vitro antologous tumor assay. In some embodiments, among CD4+ T cells in a provided TIL composition, greater than at or about 5% express CD107a in an in vitro autologous tumor assay. In some embodiments, among CD4+ T cells in a provided TIL composition, greater than at or about 10% express CD107a in an in vitro autologous tumor assay. In some embodiments, among CD8+ T cells in a provided TIL composition, greater than at or about 15% express CD107a in an in vitro autologous tumor assay.

In some embodiments, the TIL composition is characterized by killing tumor cells in an in vitro autologous tumor assay. In some embodiments, the TIL composition kills at least 30% of tumor cells in an in vitro autologous tumor assay. In some embodiments, the TIL composition kills at least 40% of tumor cells in an in vitro autologous tumor assay. In some embodiments, the TIL composition kills at least 50% of tumor cells in an in vitro autologous tumor assay. In some embodiments, the TIL composition kills at least 60% of tumor cells in an in vitro autologous tumor assay. In some embodiments, the TIL composition kills at least 70% of tumor cells in an in vitro autologous tumor assay. In some embodiments, the TIL composition kills at least 80% of tumor cells in an in vitro autologous tumor assay.

In certain embodiments, the number of such cells in the composition is a therapeutically effective amount. An effective amount of cells can vary depending on the patient, as well as the type, severity and extent of disease. Thus, a physician can determine what an effective amount is after considering the health of the subject, the extent and severity of disease, and other variables. In some embodiments, the amount is an amount that reduces the severity, the duration and/or the symptoms associated with cancer in an animal. In some embodiments, a therapeutically effective amount is a dose of cells that results in a reduction of the growth or spread of cancer by at least 2.5%, at least 5%, at least 10%, at least 15%, at least 25%, at least 35%, at least 45%, at least 50%, at least 75%, at least 85%, by at least 90%, at least 95%, or at least 99% in a patient or an animal administered a composition described herein relative to the growth or spread of cancer in a patient (or an animal) or a group of patients (or animals) not administered the composition. In some embodiments, a therapeutically effective amount is an amount to result in cytotoxic activity resulting in activity to inhibit or reduce the growth of cancer cells.

In some embodiments, the TIL composition provided herein enriched in tumor reactive cells comprises an amount of cells that is from at or about 10⁵ and at or about 10¹² cells. In some embodiments, the TIL composition provided herein enriched in tumor reactive cells comprises an amount of cells from at or about 10⁵ to at or about 10⁸ cells. In some embodiments, the TIL composition provided herein enriched in tumor reactive cells comprises an amount of cells from at or about 10⁶ and at or about 10¹². In some embodiments, the TIL composition provided herein enriched in tumor reactive cells comprises an amount of cells from at or about 10⁸ and at or about 10¹¹ cells. In some embodiments, the TIL composition provided herein enriched in tumor reactive cells comprises an amount of cells from at or about 10⁹ and at or about 10¹⁰ cells. In some embodiments, the TIL composition provided herein enriched in tumor reactive cells comprises an amount of greater than or greater than at or about 10⁵ cells, greater than or greater than at or about 10⁶ cells, greater than or greater than at or about 10⁷ cells, greater than or greater than at or about 10⁸ cells, greater than or greater than at or about 10⁹ cells, greater than or greater than at or about 10¹⁶ cells, greater than or greater than at or about 10¹¹ cells, or greater than or greater than at or about 10¹² cells. In some embodiments, such an amount can be administered to a subject having a disease or condition, such as to a cancer patient.

In some embodiments, the volume of the composition is at least or at least about 10 mL, 50 mL, 100 mL, 200 mL, 300 mL, 400 mL or 500 mL, such as is from or from about 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 ml or 200 mL to 500 mL, each inclusive. In some embodiments, the composition has a cell density of at least or at least about 1×10⁵ cells/mL, 5×10⁵ cells/mL, 1×10⁶ cells/mL, 5×10⁶ cells/mL, 1×10⁷ cells/mL, 5×10⁷ cells/mL or 1×10⁸/cells/mL. In some embodiment, the cell density of the composition is between or between about 1×10⁵ cells/mL to 1×10⁸ cells/mL, 1×10⁵ cells/mL to 1×10⁷ cells/mL, 1×10⁵ cells/mL to 1×10⁶ cells/mL, 1×10⁶ cells/mL to 1×10⁷ cells/mL, 1×10⁶ cells/mL to 1×10⁸ cells/mL, 1×10⁶ cells/mL to 1×10⁷ cells/mL or 1×10⁷ cells/mL to 1×10⁸ cells/mL, each inclusive.

Among the compositions are pharmaceutical compositions and formulations for administration, such as for adoptive cell therapy. In some embodiments, the cells are formulated with a pharmaceutically acceptable carrier.

A pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringe″s solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for cells, such 's a saline solution, a dextrose solution or a solution comprising human serum albumin.

In some embodiments, the pharmaceutically acceptable carrier or vehicle for such compositions is any non-toxic aqueous solution in which the cells can be maintained, or remain viable, for a time sufficient to allow administration of live cells. For example, the pharmaceutically acceptable carrier or vehicle can be a saline solution or buffered saline solution. The pharmaceutically acceptable carrier or vehicle can also include various bio materials that may increase the efficiency of cells. Cell vehicles and carriers can, for example, include polysaccharides such as methylcellulose (M. C. Tate, D. A. Shear, S. W. Hoffman, D. G. Stein, M. C. LaPlaca, Biomaterials 22, 1113, 2001, which is incorporated herein by reference in its entirety), chitosan (Suh J K F, Matthew H W T. Biomaterials, 21, 2589, 2000; Lahiji A, Sohrabi A, Hungerford D S, et al., J Biomed Mater Res, 51, 586, 2000, each of which is incorporated herein by reference in its entirety), N-isopropylacrylamide copolymer P(NIPAM-co-AA) (Y. H. Bae, B. Vernon, C. K. Han, S. W. Kim, J. Control. Release 53, 249, 1998; H. Gappa, M. Baudys, J. J. Koh, S. W. Kim, Y. H. Bae, Tissue Eng. 7, 35, 2001, each of which is incorporated herein by reference in its entirety), as well as Poly(oxyethylene)/poly(D, L-lactic acid-co-glycolic acid) (B. Jeong, K. M. Lee, A. Gutowska, Y. H. An, Biomacromolecules 3, 865, 2002, which is incorporated herein by reference in its entirety), P(PF-co-EG) (Suggs L J, Mikos A G. Cell Trans, 8, 345, 1999, which is incorporated herein by reference in its entirety), PEO/PEG (Mann B K, Gobin A S, Tsai A T, Schmedlen R H, West J L., Biomaterials, 22, 3045, 2001; Bryant S J, Anseth K S. Biomaterials, 22, 619, 2001, each of which is incorporated herein by reference in its entirety), PVA (Chih-Ta Lee, Po-Han Kung and Yu-Der Lee, Carbohydrate Polymers, 61, 348, 2005, which is incorporated herein by reference in its entirety), collagen (Lee C R, Grodzinsky A J, Spector M., Biomaterials 22, 3145, 2001, which is incorporated herein by reference in its entirety), alginate (Bouhadir K. H, Lee K Y, Alsberg E, Damm K L, Anderson K W, Mooney D J. Biotech Prog 17, 945, 2001; Smidsrd O, Skjak-Braek G., Trends Biotech, 8, 71, 1990, each of which is incorporated herein by reference in its entirety).

In some embodiments, the composition, including pharmaceutical composition, is sterile. In some embodiments, isolation or enrichment of the cells is carried out in a closed or sterile environment, for example, to minimize error, user handling and/or contamination. In some embodiments, sterility may be readily accomplished, e.g., by filtration through sterile filtration membranes.

Also provided herein are compositions that are suitable for cryopreserving the provided T cells, including tumor-reactive T cells. In some embodiments, the composition comprises a cryoprotectant. In some embodiments, the cryoprotectant is or comprises DMSO and/or glycerol. In some embodiments, compositions formulated for cryopreservation can be stored at low temperatures, such as ultra low temperatures, for example, storage with temperature ranges from −40° C. to −150° C., such as or about 80° C.±6.0° C.

Also provided herein is a frozen composition containing any of the provided TIL compositions and a cryoprotectant.

In some embodiments, the cryopreserved cells are prepared for administration by thawing. In some cases, the cells can be administered to a subject immediately after thawing. In such an embodiment, the composition is ready-to-use without any further processing. In other cases, the cells are further processed after thawing, such as by resuspension with a pharmaceutically acceptable carrier, incubation with an activating or stimulating agent, or are activated washed and resuspended in a pharmaceutically acceptable buffer prior to administration to a subject.

### II. EX VIVO METHODS OF PRODUCING TIL COMPOSITIONS ENRICHED IN TUMOR-REACTIVE T CELLS

Various embodiments of the provided methods involve the ex vivo expansion and production of a T cell therapeutic composition, particularly for use in connection with treating cancer. In some embodiments, the method of manufacturing involves the growth and manipulation of patient cells outside of the body.

In some embodiments, the provided methods for producing a TIL composition, such as any described herein, include: providing dissociated tumor cells from a tumor obtained from a donor subject, wherein the dissociated tumor cells are a first population of T cells that comprise CD4+ and CD8+ T cells; culturing the first population of T cells with recombinant IL-2 added at a concentration between 3000 IU/mL and 6000 IU/mL, inclusive, for 14 to 28 days to produce a second population of T cells; co-culturing the second population of T cells for 12 to 48 hours with autologous antigen presenting cells (APCs) with recombinant IL-2 added at a concentration of 100 IU/mL to 1000 IU/mL to produce a third population of T cells, wherein the APCs are loaded with a pool of peptide neoantigens from the tumor, wherein each peptide is 13-40 amino acids in length and is loaded at a concentration of 100 ng/mL per peptide, and wherein the ratio of the second population of T cells to APCs is 2:1 to 10:1; selecting cells from the third population of T cells that are surface positive for CD134 and/or CD137 to produce a fourth population of T cells; and expanding tumor infiltrating lymphocytes (TILs) by incubating the fourth population of T cells with irradiated human peripheral blood mononuclear cells (iPBMCs) at a ratio of 100 to 500 iPBMC to cells of the fourth population of T cells with recombinant IL-2 added at a concentration between 3000 IU/mL and 6000 IU/mL, inclusive, and 10 to 50 ng/mL anti-CD3 antibody (OKT3) for 12 to 16 days to produce a therapeutic composition of TILs enriched in tumor reactive cells.

In some embodiments methods for producing a TIL composition may include ex vivo co-culture in which the second population of T cells are incubated with APCs, such as autologous APCs, that have been exposed to or contacted with one or more peptides, e.g. synthetic peptides, under conditions in which the APCs have been induced to present one or more peptides from a tumor-associated antigen. In some embodiments, the population of T cells are autologous T cells from a subject with a tumor and the source of synthetic peptides are tumor antigenic peptides from a tumor antigen of the same subject. In some embodiments, cells from the ex vivo co-culture are a population of cells (third population) that include tumor reactive T cells that recognize or are activated by a peptide presented on an MHC of an APC in the culture. In some embodiments, cells from the ex vivo co-culture represent a source of cells that are enriched for tumor reactive T cells.

In some cases, the tumor reactive T cells can be further enriched by separation or selection of cells that express one CD137, CD134 or CD137 and CD134 (also phrased “CD134 and/or CD137”).

In particular embodiments, a second expansion (also called “rapid expansion protocol” or REP) is performed on T cells enriched or isolated from the co-culture, such as after separation or selection of tumor reactive T cells for cells that express or are surface positive for CD134 and/or CD137. The second expansion involves incubation to further stimulate T cells for expansion with anti-CD3 antibody (e.g. OKT3), irradiated peripheral blood mononuclear cells (iPBMCs), and recombinant IL-2. The T cells are allowed to expand for a certain number of days as desired and/or until a therapeutic dose or harvest dose is met. The composition of expanded T cells can then be harvested and formulated for administration to a subject for treatment of a cancer in the subject.

In particular embodiments, the incubation or culture of T cells also is carried out with nutrient containing media so that the cells can survive outside of the body. In embodiments of the provided methods, one or more of the steps can be carried out in serum-containing media, such as media containing human AB serum. In embodiments of the provided methods, one or more of the steps can be carried out in serum-free media. In one embodiment, the serum free medium is OpTmizer CTS (LifeTech), Immunocult XF (Stemcell technologies), CellGro (CellGenix), TexMacs (Miltenyi), Stemline (Sigma), Xvivo15 (Lonza), PrimeXV (Irvine Scientific), or Stem×Vivo (RandD systems). The serum-free medium can be supplemented with a serum substitute such as ICSR (immune cell serum replacement) from LifeTech. The level of serum substitute (e.g., ICSR) can be, e.g., up to 5%, e.g., about 1%, 2%, 3%, 4%, or 5%. In some embodiments, the serum-free media contains 0.5 mM to 5 mM of a dipeptide form of L-glutamine, such L-alany 1-L-glutamine (Glutamax™). In some embodiments, the concentration of the dipeptide form of L-glutamine, such as L-alanyl-L-glutamine, is from or from about 0.5 mM to 5 mM, 0.5 mM to 4 mM, 0.5 mM to 3 mM, 0.5 mM to 2 mM, 0.5 mM to 1 mM, 1 mM to 5 mM, 1 mM to 4 mM, 1 mM to 3 mM, 1 mM to 2 mM, 2 mM to 5 mM, 2 mM to 4 mM, 2 mM to 3 mM, 3 mM to 5 mM, 3 mM to 4 mM or 4 mM to 5 mM, each inclusive. In some embodiments, the concentration of the dipeptide form of L-glutamine, such as L-alany 1-L-glutamine, is or is about 2 mM.

### A. Neoantigen Identification and Peptide Generation

The provided methods include a step of generating or identifying in silico a plurality of peptides (also referred to as “P” or “n-mers”) that contain at least one cancer-specific cancer neoantigen. In some embodiments, at least one synthetic peptide is prepared using sequence information from all or a subset of neoantigen sequences, and the synthetic peptide is then employed in methods to enrich for tumor-reactive T cells in accord with the provided methods.

In some embodiments, the cancer-specific cancer neoepitope is determined by identifying or isolating a tumor-associated antigen or peptide sequence thereof from a cancer cell from a subject. The cancer cell may be obtained from any bodily sample derived from a patient which contains or is expected to contain tumor or cancer cells. The bodily sample may be any tissue sample such as blood, a tissue sample obtained from the primary tumor or from tumor metastases, a lymph node sample or any other sample containing tumor or cancer cells.

In some embodiments, the tumor is a hematological tumor. Non-limiting examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstron's macroglobulinemia, heavy chain disease, myelodysplastic syndrome, hairy cell leukemia and myelodysplasia.

In some embodiments, the tumor is a solid tumor. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewin's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilm” tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the tumor is from a patient with a cancer, including, but not limited to, ovarian, vulva, endometrial, urothelial, breast, colorectal, lung, renal, and skin (including but not limited to melanoma). In some embodiments, the tumor is from a patient with an ovarian cancer. In some embodiments, the tumor is from a patient with cancer of the vulvar. In some embodiments, the tumor is from a patient with an endometrial cancer. In some embodiments, the tumor is from a patient with a urothelial cancer. In some embodiments, the tumor is from a patient with a breast cancer. In some embodiments, the tumor is from a patient with a colorectal cancer. In some embodiments, the tumor is from a patient with a lung cancer. In some embodiments, the tumor is from a patient with a renal cancer. In some embodiments, the tumor is from a patient with melanoma. In particular embodiments, the tumor is from a patient to be treated as described in Section III.

In some embodiments, the cancer is a gastrointestinal cancer involving a cancer of the gastrointestinal tract (GI tract), including cancers or the upper or lower digestive tract, or an accessory organ of digestion, such as esophagus, stomach, biliary system, pancreas, small intestine, large intestine, rectum or anus. In some embodiments, the cancer is an esophageal cancer, stomach (gastric) cancer, pancreatic cancer, liver cancer (hepatocellular carcinoma), gallbladder cancer, cancer of the mucosa-associated lymphoid tissue (MALT lymphoma), cancer of the biliary tree, colorectal cancer (including colon cancer, rectum cancer or both), anal cancer, or a gastrointestinal carcinoid tumor.

In particular embodiments, the cancer is a colorectal cancer.

In some embodiments, the tumor is from a breast cancer, such as a ductal carcinoma or a lobular carcinoma. In some embodiments, the tumor is from a prostate cancer. In some embodiments, tumor is from a skin cancer, such as a basal cell carcinoma, a squamous cell carcinoma, a Kaposi's sarcoma, or a melanoma. In some embodiments, the tumor is from a lung cancer, such as an adenocarcinoma, a bronchiolaveolar carcinoma, a large cell carcinoma, or a small cell carcinoma. In some embodiments, the tumor is from a brain cancer, such as a glioblastoma or a meningioma. In some embodiments, the tumor is from a gastrointestinal cancer, such as any described above. In some embodiments, the tumor is from a colon cancer. In some embodiments, the tumor is from a liver cancer, such as a hepatocellular carcinoma. In some embodiments, the tumor is from a pancreatic cancer. In some embodiments, the tumor is from a kidney cancer, such as a renal cell carcinoma. In some embodiments, the tumor is from a testicular cancer.

In some embodiments, the cancer is not a melanoma. Melanoma is a cancer that generally has a high mutational rate. High tumor mutation burden has been thought to be a particularly desired prognostic marker for success related to treatment with an immunotherapy targeting tumor neoantigens (Simpson et al., Journal of Clinical Oncology 2017, 35:15_suppl, 9567-9567; McGranahan et al. Science 2016, 351:1463-1469) In some embodiments, the provided methods can be used in cancers that have a lower tumor mutation burden, since the methods are carried out to actively (as opposed to passively) enrich for tumor reactive T cells.

In some embodiments, the subject has a tumor mutational burden of 5 to 6000 mutations. In some embodiments, the subject has a tumor mutational burden of 100 to 5500 mutations. In some embodiments, the subject has a tumor mutations burden of about 100 mutations, 200 mutations, 300 mutations, 400 mutations, 500 mutations, 600 mutations, 700 mutations, 800 mutations, 900 mutations, 1000 mutations, 1500 mutations, 2000 mutations, 2500 mutations, 3000 mutations, 3500 mutations, 4000 mutations, 4500 mutations, 5000 mutations, 5500 mutations or any value between any of the foregoing.

In some embodiments, the subject is a subject with a tumor mutational burden (TMB) of less than 8 mutations.

In some embodiments, TMB includes the number of non-synonymous mutations per tumor. In some embodiments, TMB can be calculated by counting the number of synonymous and non-synonymous mutations across a 0.8- to 1.2-megabase (Mb) region, and reporting the result as mutations/Mb. In some embodiments, TMB can be determined by next generation sequencing (NGS) on tumor tissue samples. In some cases, whole exome sequencing can be used or computational germline status filtering can be used (Chalmers et al. Genome Med 2017 9:34), In some embodiments, the subject has a TMB of less than at or about 60 mutations/Mb, such as less than at or about 55 mutations/Mb, less than at or about 50 mutations/Mb, less than at or about 45 mutations/Mb, less than at or about 40 mutations/Mb, less then at or about 30 mutations/Mb, less than at or about 25 mutations per Mb, or less than at or about 20 mutations/Mb, or any value between any of the foregoing. In some embodiments, the subject has a TMB of less than at or about 41 mutations/Mb, less than at or about 40 mutations/Mb, less than at or about 39 mutations/Mb, less than at or about 38 mutations/Mb, less than at or about 37 mutations/Mb or less.

In some embodiments, the peptide (P) is a tumor-associated antigen derived from premalignant conditions, such as variants of carcinoma in situ, or vulvar intraepithelial neoplasia, cervical intraepithelial neoplasia, or vaginal intraepithelial neoplasia.

In some aspects, nucleic acid from such cells of the tumor or cancer is obtained and sequenced. In embodiments, the protein-coding region of genes in a genome is obtained, such as by omics analysis, such as by analysis of whole genomic sequencing data, exome sequencing data, and/or transcriptome data. To identify tumor-specific sequences, sequencing data can be compared to a reference sequencing data, such as data obtained from a normal cell or noncancerous cell from the same subject. In some embodiments, next-generation sequencing (NGS) methods are used.

In some embodiments, the methods include a step of using matched normal omics data of a tumor. In such methods, the in silico analysis involves an omics analysis to identify mutations in the tumor relative to normal tissue of the same patient, such as non-diseased tissue of the same patient. It is generally contemplated that matched normal omics data are whole genomic sequencing data, exome sequencing data, and/or transcriptome data, and that the matched normal omics data are matched against normal before treatment of the patient. In a particular embodiment, whole exome sequencing is performed on healthy and diseased tissue to identify somatic mutations associated with the tumor.

In some embodiments, omics data are obtained from one or more patient biopsy samples following standard tissue processing protocol and sequencing protocols. In particular embodiments, the data are patient matched tumor data (e.g., tumor versus same patient normal). In some cases, non-matched or matched versus other reference (e.g., prior same patient normal or prior same patient tumor, or homo statisticus) are also deemed suitable for use herein. The omics data may be fresh omics data or omics data that were obtained from a prior procedure (or even different patient). For example, neoepitopes may be identified from a patient tumor in a first step by whole genome and/or exome analysis of a tumor biopsy (or lymph biopsy or biopsy of a metastatic site) and matched normal tissue (i.e., non-diseased tissue from the same patient such as peripheral blood). In some embodiments, genomic analysis can be processed via location-guided synchronous comparison of the so obtained omics information.

The genomic analysis can be performed by any number of analytic methods. In particular embodiments, the methods include WGS (whole genome sequencing) and exome sequencing of both tumor and matched normal sample using next generation sequencing such as massively parallel sequencing methods, ion torrent sequencing, pyrosequencing. Computational analysis of the sequence data may be performed in numerous manners. In some embodiments, the data format is in SAM, BAM, GAR, or VCF format. As an example, analysis can be performed in silico by location-guided synchronous alignment of tumor and normal samples as, for example, disclosed in US 2012/0059670A1 and US 2012/0066001 A1 using BAM files and BAM servers. Alternative file formats for sequence analysis (e.g., SAM, GAR, FASTA, etc.) are also contemplated.

In some of any embodiments, peptides (P) comprising neoantigens arising from a missense mutation encompass the amino acid change encoded by 1 or more nucleotide polymorphisms. Peptides (P) comprising neoantigens that arise from frameshift mutations, splice site variants, insertions, inversions and deletions should encompass the novel peptide sequences and junctions of novel peptide sequences. Peptides (P) comprising neoantigens with novel post-translational modifications should encompass the amino acids bearing the post-translational modification(s), such as a phosphate or glycan.

Once these mutations are identified, neoepitopes are then identified. Neoepitopes are mutant peptides that are recognized by a patient's T cells. These neoepitopes must be presented by a tumor or antigen presenting cell by the MHC complex and then be recognized by a TCR on the T cell. In some embodiments, the provided methods include a step of calculation of one or more neoepitopes to define neoepitopes that are specific to the tumor and patient. Consequently, it should be recognized that patient and cancer specific neoepitopes can be identified from omics information in an exclusively in silico environment that ultimately predicts potential epitopes that are unique to the patient and tumor type. In particular aspects, the so identified cancer neoepitopes are unique to the patient and the particular cancer in the patient (e.g., having a frequency of less than 0.1% of all neoepitopes, and more typically less than 0.01% in a population of cancer patients diagnosed with the same cancer), but that the so identified cancer neoepitopes have a high likelihood of being presented in a tumor.

In some of any embodiments, the length of the peptide (P) depends on the specific application and is typically between about 5 to about 50 amino acids. In particular embodiments, neoepitopes will be calculated to have a length of between 2-50 amino acids, such as 13-40 amino acids, for example 25 amino acids (25mer). In preferred embodiments, the peptide (P) is between about 13 to 40 amino acids, e.g., 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

In some embodiments, the methods are carried out with a pool of peptides. The pool of peptides can include tens to hundreds of individual peptides. The pool of peptides can include up to 200 different peptides containing predicted mutations. In some cases, the pool of peptides includes 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150, 160, 170, 180, 190 or more individual peptides, or any value between any of the foregoing.

The pool of peptides can represent one neo-antigen or can represent several neo-antigens. In some cases, a pool of peptides can include multiple overlapping peptides of the same neo-antigen. Thus, for a tumor-associated antigen, the antigen may be divided into 13 to 40 amino acid, e.g., 25 amino acid, peptides (P) wherein each peptide (P) contains a unique composition of amino acids; or, the peptides (P) can be overlapping peptide pools wherein an antigen is divided into a set number of 13 to 40 amino acid, e.g., 25 amino acid, peptides (P) that have overlapping sequences. In some cases, each of the peptides of the overlapping pool of an antigen can be offset by a set number of amino acid residues, such as 5, 6, 7, 8, 9, 10, 11, 12, 13, 15 or 15 amino acids. In some embodiments, each of the peptides of the overlapping pool of an antigen is offset by 10 amino acids. In some embodiments, each of the peptides of the overlapping pool of an antigen is offset by 12 amino acids. For example, an overlapping peptide pool comprising a 100 amino acid antigen may be divided into eight 25 amino acid peptides (P) that are each offset by 12 amino acids (i.e., each subsequent 25 amino acid peptide comprising a 100 amino acid peptide sequence starts at the 13^{th} amino acid position from the prior peptide). Those skilled in the art understand that many permutations exist for generating a peptide pool from an antigen.

Various algorithms have been developed and can be used to map T cell epitopes (both MHC Class I and Class II-restricted) within protein molecules of various origins. In some embodiments, many programs utilize availability of the large-scale peptide-MHC binding affinity matrix from experimental measurements to train machine learning (ML)-based classifiers to distinguish MHC-binders from non-binders (see e.g., Zhao et al. (2018) pLoS Comput Biol 14(11): e1006457). Exemplary predictor methods for MHC class I (e.g. 9-mer) include smm, smmpmbec, ann (NetMHC3.4), NetMHC4, PickPocket, consensus, NetMHCpan2.8, NetMHCpan3, NetMHCpan4, NetMHCcons, mheflurry, mheflurry_pan, or MixMHCpred. Exemplary predictor methods for MHC class II (e.g. 15-mer) include NetMHCIIpan, NetMHCII2.3, nn_align, smm_align, consensus, comblib, tepitope, or mbeflurry. Any of such methods can be used.

After the in silico identification of suitable neoepitope sequences, corresponding synthetic peptides are then prepared in vitro (e.g., using solid phase synthesis). In particular embodiments, a library of synthetic peptides is prepared representing a plurality of different neoepitopes from the subject.

Various methods can be used to prepare synthetic peptides. For example, peptides with cancer neoepitope sequences can be prepared on a solid phase (e.g., using Merrified synthesis), via liquid phase synthesis, or from smaller peptide fragments. Peptide epitopes can be obtained by chemical synthesis using a commercially available automated peptide synthesizer. In some embodiments, the peptides can be synthesized, for example, by using the Fmoc-polyamide mode of solid-phase peptide synthesis which is disclosed by Lu et al (1981). J. Org. Chem. 46,3433 and the references therein. In some aspects, peptides can be produced by expression of a recombinant nucleic acid in a suitable host and with a suitable expression system. In some aspects, recombinant methods can be used where multiple neoepitopes are on a single peptide chain, such as with spacers between neoepitopes or cleavage sites).

The peptides can be purified by any one, or a combination of techniques such as recrystallization, size exclusion chromatography, ion-exchange chromatography, hydrophobic interaction chromatography, and reverse-phase high performance liquid chromatography using e.g. acetonitrile/water gradient separation. In some embodiments, peptides can be precipitated and further purified, for example by high performance liquid chromatography (HPLC). Analysis of peptides can be carried out using thin layer chromatography, electrophoresis, in particular capillary electrophoresis, solid phase extraction (CSPE), reverse-phase high performance liquid chromatography, amino-acid analysis after acid hydrolysis and by fast atom bombardment (FAB) mass spectrometric analysis, as well as MALDI and ESI-Q-TOF mass spectrometric analysis.

### B. Tumor Sample for First Expansion

The provided methods include obtaining and enriching or selecting a population of T cells from a biological sample for use as a first population of T cells (also called input population). In some cases, the first population of T cells is one that is known or likely to contain T cells reactive to a tumor antigen or that are capable of being reactive to a tumor antigen, such as following an ex vivo co-culture with an autologous source of tumor antigen. For example, typically the first population of T cells is from a biological sample from a tumor or from a subject known or likely to have a tumor. In particular embodiments, the first population of T cells is further stimulated with one or more T cell stimulatory agent(s) (e.g. one or more recombinant cytokines, such as IL-2) to produce a second or stimulated population of T cells containing T cells that have expanded following the stimulation.

In some embodiments, the sample is a tumor sample and thereby provides a source of tumor-infiltrating lymphocytes (TILs). In some aspects, TILs are T cells that have left the bloodstream of a subject and migrated into or infiltrated a tumor. In particular aspects, TILs are reactive to a tumor antigen.

A patient tumor sample may be obtained by any of a variety of methods in which the method obtains a sample that contains a mixture of tumor and TIL cells. In some embodiments, the tumor sample is obtained by surgical resection. In some embodiments, the tumor sample is obtained by needle biopsy. In general, the tumor sample may be from any solid tumor, including primary tumors, invasive tumors or metastatic tumors. The tumor sample may also be a liquid tumor, such as a tumor obtained from a hematological malignancy. Typically the tumor sample is from the same patient as the tumor sample used for neoantigen identification as described above. In some embodiments, the tumor sample is the same tumor sample as used for neoantigen identification as described above.

In some embodiments, the tumor sample is from a solid tumor that may be of any cancer type, including, but not limited to, ovarian, vulva, endometrial, urothelial, breast, pancreatic, prostate, colorectal, lung, brain, renal, stomach (gastrointestinal), and skin (including but not limited to squamous cell carcinoma, basal cell carcinoma, and melanoma). In some embodiments, the tumor is from a patient with a cancer, including, but not limited to, ovarian, vulva, endometrial, urothelial, breast, colorectal, lung, renal, and skin (including but not limited to melanoma). In some embodiments, the tumor is from a patient with an ovarian cancer. In some embodiments, the tumor is from a patient with cancer of the vulvar. In some embodiments, the tumor is from a patient with an endometrial cancer. In some embodiments, the tumor is from a patient with a urothelial cancer. In some embodiments, the tumor is from a patient with a breast cancer. In some embodiments, the tumor is from a patient with a colorectal cancer. In some embodiments, the tumor is from a patient with a lung cancer. In some embodiments, the tumor is from a patient with a renal cancer. In some embodiments, the tumor is from a patient with melanoma. In particular embodiments, the tumor is from a patient to be treated as described in Section III.

In particular embodiments, a T cell population is one that includes both CD4+ and CD8+ T cells. Many cancers, including solid tumors, such as many common epithelial indications (e.g. GI), express class I and class II restricted mutations. In order for a T cell product to target such indications, e.g. common epithelial indications, it is contemplated that both CD8+ T cells to recognize class I MHC-restricted molecules and CD4+ T cells to recognize Class II MHC-restricted molecules are necessary.

The sample may be obtained from a variety of different subjects/patients/hosts. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

In some aspects, the subject is a human. Accordingly, the cells in some embodiments are primary cells, e.g., primary human cells. In some embodiments, the sample is autologous to a subject to be treated, such as a subject who is a patient in need of a particular therapeutic intervention, such as the adoptive cell therapy for which cells are being isolated, processed, and/or expanded in accord with the provided methods. In some embodiments, the sample is allogenic to a subject to be treated.

In provided embodiments, the obtained tumor sample is fragmented into small pieces of between at or about 1 mm³ and at or about 8 mm³ in size, such as between at or about 1 mm³ and at or about 3 mm³, between at or about 1 mm³ and at or about 4 mm³, between at or about 1 mm³ and at or about 2 mm³. In some embodiments, the tumor fragment is from about −3 mm³. In some embodiments, the tumor fragment is from about 1-3 mm³. In some embodiments, the tumor fragment is obtained by physical fragmentation, such as by dissection. In some embodiments, the tumor fragment is obtained by sharp dissection.

In some of any of the provided embodiments, the obtained tumor sample is fragmented into small pieces of between at or about 1 mm and at or about 8 mm in diameter, such as between at or about 1 mm and at or about 6 mm in diameter, between at or about 1 mm and at or about 4 mm in diameter, between at or about 1 mm and at or about 2 mm in diameter. In some embodiments, the tumor fragment is from about 3 mm in diameter. In some embodiments, the tumor fragment is from about 1-2 mm in diameter. In some embodiments, the tumor fragment is obtained by physical fragmentation, such as by dissection. In some embodiments, the tumor fragment is obtained by sharp dissection.

In some embodiments, the tumor sample is cryopreserved prior to fragmentation. In some embodiments, the tumor fragments are cryopreserved.

In some embodiments, tumor fragments are used as a source to prepare a single cell suspension for use as an input population of T cells in the first expansion in the provided methods.

In some embodiments, the provided methods involve obtaining cells from the tumor fragments, such as by enzymatic digestion of tumor fragments to obtain TILs. Enzymatic digestion can be carried out using a collagenase, such as a type IV collagenase or a type I/II collagenase. The enzyme, such as a collagenase, can be present in media for the enzymatic digestion at a concentration of from at or about 1 mg/mL to at or about 5 mg/mL, such as at or about 1 mg/mL, at or about 2 mg/mL, at or about 3 mg/mL, at or about 4 mg/mL or at or about 5 mg/mL, or any value between any of the foregoing. The enzyme, such as a collagenase, can be present in media for the enzymatic digestion at a concentration of from at or about 5 mg/mL to at or about 10 mg/mL, such as at or about 5 mg/mL, at or about 6 mg/mL, at or about 7 mg/mL, at or about 8 mg/mL, at or about 9 mg/mL, or at or about 10 mg/mL, or any value between any of the foregoing. In some embodiments, the concentration is about 5 mg/mL. In some embodiments, the concentration is 10 mg/mL. In some embodiments, the collagenase is a type IV collagenase. In some embodiments, the collagenase is a type I/II collagenase. In some embodiments, the enzymatic digestion is with a media that includes type IV collagenase, such as from at or about 1 mg/mL to at or about 5 mg/mL. In some embodiments, the enzymatic digestion is with a media that includes type I/II collagenase, such as from at or about 1 mg/mL to at or about 5 mg/mL.

In some embodiments, enzymatic digestion can be carried out, in part, using a hyaluronidase. Hyaluronidase is a hyaluronic acid-metabolizing enzyme, subsequently enhancing cell membrane permeability (Eikenes et al. Anticancer Research, 2010). The enzyme, such as a hyaluronidase, can be present in media for the enzymatic digestion at a concentration of from at or about 5 mg/mL to at or about 10 mg/mL, such as at or about 5 mg/mL, at or about 6 mg/mL, at or about 7 mg/mL, at or about 8 mg/mL or at or about 9 mg/mL, at or about 10 mg/mL or any value between any of the foregoing. In some embodiments, the enzymatic digestion is with a media that includes type II hyaluronidase, such as from at or about 5 mg/mL to at or about 10 mg/mL. In some embodiments, if a more gentle digestion is desired at or about 5 mg/mL hyaluronidase is used. In some embodiments, if a more complete digestion is desired a higher concentration of hyaluronidase is used, such as at or about 10 mg/ml hyaluronidase.

In some embodiments, dNase is also present in the media during enzymatic digestion. dNase is an enzyme that degrades any free DNA released into the media as a result of the tumor fragment digestion process. The enzyme, such as a dNase I, can be present in media for the enzymatic digestion at a concentration of from at or about 5,000 units/mL to at or about 10,000 units/mL, such as at or about 5,000 units/mL, at or about 6,000 units/mL, at or about 7,000 units/mL, at or about 8,000 units/mL or at or about 9,000 units/mL, at or about 10,000 units/mL or any value between any of the foregoing. In some embodiments, the enzymatic digestion is with a media that includes dNase I, such as from at or about 5,000 units/mL to at or about 10,000 units/mL.

In other embodiments, enzymes from the Miltenyi human tumor dissociation kit can be used (e.g. Cat. O. 130-095-929; Miltenyi Biotec). The enzymatic media containing the enzyme can be a serum-free media, such as any as described. In particular embodiments, enzymatic media includes collagenase, e.g., Roswell Park Memorial Institute (RPMI) 1640 buffer, 2 mM glutamate (e.g. GlutaMAX), 10 mg/mL gentamicin, 30 units/mL of dNase and 1.0 mg/mL of collagenase). In some embodiments, enzymatic media includes a serum free media (e.g. OpTmizer) containing 2 mM glutamate (e.g. GlutaMAX), 10 μg/mL gentamicin, an immune cell serum replacement (e.g. CTS Immune Cell Serum Replacement) and 1.0 mg/mL to 5.0 mg/ml of collagenase). In particular embodiments, enzymatic media includes hyaluronidase and/or collagenase, e.g., Roswell Park Memorial Institute (RPMI) 1640 buffer, 2 mM glutamate (e.g. GlutaMAX), 10 mg/ml gentamicin, 10,000 units/mL of dNase I, 10 mg/ml of collagenase and 10 mg/ml of hyaluronidase).

The tumor fragment is then mechanically dissected to dissociate the TILs, e.g., using a tissue dissociator. An example of a tissue dissociator is GentleMACs™ (Miltenyi Biotec) to homogenize the tissue. Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. In some embodiments, tumor digests are subjected to homogenization and enzymatic digestion by incubation in the enzyme cocktail for 15 minutes to 2 hours, such as for at or about 30 minutes to 60 minutes. In some embodiments, tumor digests are subjected to homogenization and enzymatic digestion by incubation in the enzyme cocktail for about 60 minutes. At the end of this process, if the cell suspension contains a large number of red blood cells or dead cells, a density gradient separation using FICOLL can be performed to remove these cells. In some embodiments, a single cell suspension is prepared following processing of the tumor fragments by straining the cells through a filter to remove debris, such as a 70 μm strainer. In some cases, separation can be achieved by centrifugation, in which case the cell pellet can be resuspended and strained through a e.g. 70 μm strainer to remove debris. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of obtaining TILs for use in the provided methods.

In some embodiments, a single cell suspension for use as an input population comprises from at or about 1×10⁶ dissociated tumor cells to at or about 1000×10⁶ dissociated tumor cells, such as 1×10⁶ to 500×10⁶ dissociated tumor cells, 1×10⁶ to 100×10⁶ dissociated tumor cells, 1×10⁶ to 50×10⁶ dissociated tumor cells, 1×10⁶ to 10×10⁶ dissociated tumor cells, 10×10⁶ to 1000×10⁶ dissociated tumor cells, 10×10⁶ to 100×10⁶ dissociated tumor cells, 10×10⁶ to 500×10⁶ dissociated tumor cells, 10×10⁶ to 50×10⁶ dissociated tumor cells, 50×10⁶ to 1000×10⁶ dissociated tumor cells, 50×10⁶ to 500×10⁶ dissociated tumor cells, 50×10⁶ to 100×10⁶ dissociated tumor cells, 100×10⁶ to 1000×10⁶ dissociated tumor cells, 100×10⁶ to 500×10⁶ dissociated tumor cells, or 500×10⁶ to 1000×10⁶ dissociated tumor cells. In some embodiments, a single cell suspension for use as an input population of T cells comprises from at or about or at least at or about 10×10⁶ dissociated tumor cells, 20×10⁶ dissociated tumor cells, 30×10⁶ dissociated tumor cells, 40×10⁶ dissociated tumor cells, 50×10⁶ dissociated tumor cells, 60×10⁶ dissociated tumor cells, 70×10⁶ dissociated tumor cells, 80×10⁶ dissociated tumor cells, 90×10⁶ dissociated tumor cells, or 100×10⁶ dissociated tumor cells In some embodiments, a single cell suspension for use as an input population of T cells comprises from at or about 10×10⁶ dissociated tumor cells to at or about 100×10⁶ dissociated tumor cells.

In some embodiments, digested cells from the tumor fragments are placed into culture media under conditions and with appropriate nutrients to mediate T cell activation and/or sustain T cell expansion, such as any of the conditions described below for stimulation and pre-expansion of T cells. In some embodiments, the T cell stimulation includes incubating the first population of T cells generated or obtained above (e.g. dissociated tumor cells) with one or more T cell stimulatory agent(s) (e.g. one or more recombinant cytokines, such as IL-2) to produce a second or stimulated population of T cells containing T cells that have expanded following the stimulation. The cells are seeded at a particular density suitable for the particular culture vessel. The culture vessel can be a microwell, flask, tube, bag or other closed system device. In some embodiments the culture vessel is a closed container that provides a gas-permeable surface area, such as a gas permeable flask. An exemplary culture vessel that provides a gas-permeable surface area include G-Rex plates or flasks. For a G-Rex 6M (single well) or 10M vessel, it is ideal to inoculate with between 1×10⁷ and 4×10⁷ total cells. The particular culture vessel can be chosen based on the number of cells available and/or the desired yield of cells. The choice of culture vessel (e.g. G-Rex) can be chosen by linearly scaling the number of cells seeded to the surface area of the culture vessel. In some embodiments, the surface area of a culture vessel is about 100 cm² (e.g. G-Rex 100 M/100M-CS)I. In some embodiments, the surface area of a culture vessel is about 500 cm² (e.g. G-Rex 500 M/500M-CS).

In some of any of the provided embodiments, the biological sample is a tumor sourced sample, and wherein: the number of cells at the initiation of the culturing is between at or about 10×10⁶ and 100×10⁶ total viable cells, 20×10⁶ and 100×10⁶ total viable cells, or 12×10⁶ and 43×10⁶ total viable cells; or is at or about 10×10⁶ total viable cells, at or about 12×10⁶ total viable cells, 20×10⁶ total viable cells, 40×10⁶ total viable cells, 60×10⁶ total viable cells, or 100×10⁶ total viable cells,, or any value between any of the foregoing.

In some embodiments, IL-2 is added to the culture media for expansion. In some embodiments, the culture media is a complete media. In some embodiments, the culture media is a serum free media. In some embodiments, the culture media is a serum-free media containing recombinant IL-2.

In some embodiments, recombinant IL-2 is present in the cell culture medium. IL-2 is a cytokine that supports T cell recovery and proliferation. IL-2 also supports the homeostasis of T cells, thereby supporting their phenotype, differentiation status, and immune memory. In some cases, induction of regulatory T cells in the tumor microenvironment may lead to low bioavailability of IL-2. Recombinant IL-2 has been regularly used in broad expansion of T cells in various contexts. Recombinant IL-2 is commercially available. In particular embodiments, recombinant IL-2 is GMP grade (e.g. MACS GMP Recombinant Human IL-2, Miltenyi Biotec).

In some embodiments, the recombinant IL-2 is added to the culture medium at a concentration between at or about 1000 IU/mL at or about 8000 IU/mL, such as between at or about 1000 IU/mL and at or about 7000 IU/mL, between at or about 1000 IU/mL and at or about 6000 IU/mL, between at or about 1000 IU/mL and at or about 5000 IU/mL, between at or about 1000 IU/mL and at or about 4000 IU/mL, between at or about 1000 IU/mL and at or about 2000 IU/mL, 2000 IU/mL at or about 8000 IU/mL, between at or about 2000 IU/mL and at or about 7000 IU/mL, between at or about 2000 IU/mL and at or about 6000 IU/mL, between at or about 2000 IU/mL and at or about 5000 IU/mL, between at or about 2000 IU/mL and at or about 4000 IU/mL, 4000 IU/mL at or about 8000 IU/mL, between at or about 4000 IU/mL and at or about 7000 IU/mL, between at or about 4000 IU/mL and at or about 6000 IU/mL, between at or about 4000 IU/mL and at or about 5000 IU/mL, between at or about 5000 IU/mL at or about 8000 IU/mL, between at or about 5000 IU/mL and at or about 7000 IU/mL, between at or about 5000 IU/mL and at or about 6000 IU/mL, between at or about 6000 IU/mL at or about 8000 IU/mL, between at or about 6000 IU/mL and at or about 7000 IU/mL or between at or about 7000 IU/mL and at or about 8000 IU/mL. In some embodiments, recombinant IL-2 is present in an amount that is or is about 6000 IU/mL. In some embodiments, recombinant IL-2 is present in an amount that is or is about 3000 IU/mL.

In some embodiments, the incubation with the T cell stimulatory agent(s) is carried out under conditions for initial expansion of T cells from the biological sample. In some embodiments, the cells are cultured at about 37° C. with about 5% CO₂.

In some embodiments, the incubation with the T cell stimulatory agent(s) is carried out for 7 to 28 days, such as 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27days, 28 days or any value between any of the foregoing. In some embodiments, the incubation is carried out for 7-28 days. In some embodiments, the incubation is carried out for 7-14 days.

In some embodiments, the first population of T cells are cultured with recombinant IL-2 added at a concentration between 3000 IU/mL and 6000 IU/mL, inclusive, for 14 to 28 days to produce a second population of T cells. In some embodiments, the first population of T cells are cultured with recombinant IL-2 added at a concentration of about 3000 IU/mL for 14 to 28 days to produce a second population of T cells. In some embodiments, the first population of T cells are cultured with recombinant IL-2 added at a concentration of about 6000 IU/mL for 14 to 28 days to produce a second population of T cells. In some embodiments, the first population of T cells are cultured for about 14 days. In some embodiments, the first population of T cells are cultured for about 15 days. In some embodiments, the first population of T cells are cultured for about 16 days. In some embodiments, the first population of T cells are cultured for about 17 days. In some embodiments, the first population of T cells are cultured for about 18 days. In some embodiments, the first population of T cells are cultured for about 19 days. In some embodiments, the first population of T cells are cultured for about 20 days. In some embodiments, the first population of T cells are cultured for about 21 days. In some embodiments, the first population of T cells are cultured for about 22 days. In some embodiments, the first population of T cells are cultured for about 23 days. In some embodiments, the first population of T cells are cultured for about 24 days. In some embodiments, the first population of T cells are cultured for about 25 days. In some embodiments, the first population of T cells are cultured for about 26 days. In some embodiments, the first population of T cells are cultured for about 27 days. In some embodiments, the first population of T cells are cultured for about 28 days.

In some cases, media can be exchanged daily, every other day, every third day, every 5^{th} day or once a week during the time of the culture or incubation. In some embodiments, the recombinant IL-2 replenished (added) at each media exchange. In some embodiments, about 3000 IU/mL recombinant IL-2 is replenished (added) at each media exchange. In some embodiments, about 6000 IU/mL recombinant IL-2 is replenished (added) at each media exchange.

The incubation, such as for initial expansion of T cells in the biological sample, can be carried out under GMP conditions. In some embodiments, the incubation is in a closed system, which in some aspects may be a closed automated system. In some embodiments, the culture media containing the T cell stimulatory agent(s) can be a serum-free media. In some embodiments, the incubation is carried out in a closed automated system and with serum-free media.

In some embodiments, the initial expansion of cells under the one or more stimulatory conditions is in a culture vessel suitable for cell expansion. In some embodiments, the culture vessel is a gas permeable culture vessel, such as a G-Rex system (e.g. G-Rex 10, G-Rex 10M, G-Rex 100 M/100M-CS or G-Rex 500 M/500 M-CS). In some embodiments the culture vessel is a microplate, flask, bar or other culture vessel suitable for expansion of cells in a closed system. In some embodiments, expansion can be carried out in a bioreactor. In some embodiments, the initial expansion can be carried out using a cell expansion system by transfer of the cells to gas permeable bags, such as in connection with a bioreactor (e.g. Xuri Cell Expansion System W25 (GE Healthcare)). In an embodiment, the cell expansion system includes a culture vessel, such as a bag, e.g. gas permeable cell bag, with a volume that is about 50 mL, about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L, or any value between any of the foregoing. In some embodiments, the process is automated or semi-automated. Examples of suitable bioreactors for the automated perfusion expansion include, but are not limited to, GE Xuri W25, GE Xuri W5, Sartorius BioSTAT RM 20|50, Finesse SmartRocker Bioreactor Systems, and Pall XRS Bioreactor Systems, or Miltenyi Prodigy. In some aspects, the expansion culture is carried out under static conditions. In some embodiments, the expansion culture is carried out under rocking conditions. The medium can be added in bolus or can be added on a perfusion schedule. In some embodiments, the bioreactor maintains the temperature at or near 37° C. and CO2 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the culturing is performed with perfusion, such as with a rate of 290 ml/day, 580 ml/day, and/or 1160 ml/day.

In some embodiments, the cells are seeded in an appropriate culture vessel (e.g. gas permeable bag) at a density of from 0.5×10⁶ cells/mL to 1.5×10⁶ cells/mL. In some embodiments, the density is at or about 0.5×10⁶ cells/mL, 0.75×10⁶ cells/mL, 1×10⁶ cells/mL, 1.25×10⁶ cells/mL or 1.5×10⁶ cells/mL, or any value between any of the foregoing.

In some aspects, cells are expanded in an automated closed expansion system that is perfusion enabled. Perfusions can continuously add media to the cells to ensure an optimal growth rate is achieved.

In some embodiments, the stimulated cells are collected and are cryofrozen. The provision of an intermediate hold step by cryopreservation after the initial expansion phase can be used to coordinate timing with the neoepitope identification and peptide generation, such as described in Section I. A and/or the generation of APCs as described in Section I.C. In some embodiments, for cryopreservation, the stimulated cells are formulated as a composition with a cryoprotectant. In some embodiments, the cryoprotectant is or comprises DMSO and/or s glycerol. In some embodiments, compositions formulated for cryopreservation can be stored at low temperatures, such as ultra low temperatures, for example, storage with temperature ranges from −40 ° C. to −150° C., such as or about 80° C.±6.0°C.

In some embodiments, the cryopreserved cells are prepared for subsequent steps by thawing. In some cases, the cells can be ready for subsequent culturing with APCs and peptides immediately after thawing following one or more wash steps.

The expansion methods can be carried out under GMP conditions, including in a closed automated system and using serum free medium. In some embodiments, any one or more of the steps of the method can be carried out in a closed system or under GMP conditions. In certain embodiments, all process operations are performed in a GMP suite. In some embodiments, a closed system is used for carrying out one or more of the other processing steps of a method for manufacturing, generating or producing a cell therapy. In some embodiments, one or more or all of the processing steps, e.g., isolation, selection and/or enrichment, processing, culturing steps including incubation in connection with expansion of the cells, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

### C. Co-Culture With APCs

In embodiments of the provided methods, once the neoepitopes that encode for proteins are synthesized to a plurality of the synthetic peptides, a pool of synthetic peptides are contacted with antigen presenting cells under conditions to present peptides in the context of an MHC molecule. Antigen presenting cells are used to present these peptides. The peptide loaded APCs are then co-cultured with T cells from the second population of T cells that were generated after initial expansion (pre-expansion) with one or more stimulatory agents of T cells. The loaded APCs (presenting peptides) are incubated with T cells from the second population of pre-expansion T cells above for recognition of the peptides presented on the APCs. T cells that recognize these peptides on the surface of the APC can then be isolated or selected from the co-culture, such as by methods described below. In particular embodiments, co-culturing the second population of T cells is with autologous antigen presenting cells (APCs).

In some embodiments, of the provided methods the method can include co-culturing the T cells with the APCs over the course of several hours to days and then separating antigen presenting cells from the population of T cells for the expansion of the T cells under conditions to enrich or expand tumor-reactive T cells. In some embodiments, the separating can include isolating or selecting reactive T cells from culture based on one or more T cell activation markers on T cells, such as CD134 and/or CD137.

Various methods can be used for culturing cells for antigen-specificity, see e.g. US published application No. US2017/0224800.

In some embodiments, the tumor reactive T cells are co-cultured with APCs that have been contacted or exposed to present a peptide, e.g. containing a mutated amino acid sequence, such as neoepitope peptides as described above. The method may comprise inducing autologous antigen presenting cells (APCs) of the patient to present the mutated amino acid sequence. The APCs may include any cells which present peptide fragments of proteins in association with major histocompatibility complex (MHC) molecules on their cell surface. The MHC molecule can be any MHC molecule expressed by the patient including, but not limited to, MHC Class I, MHC Class II, HLA-A, HLA-B, HLA-C, HLA-DM, HLA-DO, HLA-DP, HLA-DQ, and HLA-DR molecules. The APCs may include, for example, any one or more of macrophages, DCs, Langerhans cells, B-lymphocytes, and T-cells. In particular embodiments, the APCs are DCs. In some particular embodiments, the APCs are B cells. In some embodiments, the APCs are artificial APCs.

In particular embodiments, the APCs include cells that are able to present Class I and Class II restricted molecules. For example, B cells and DCs both have the ability to present MHC class I and MHC class II restricted molecules. In some embodiments, the APC cell sample includes B cells and DCs. In some embodiments, the APC cell sample is enriched for B cells, such as by selection or isolation from a primary cell sample. In some embodiments, the APC cell sample is enriched for DCs, such as by selection or isolation from a primary cell sample.

In some embodiments, the APCs express MHC class I and/or MHC class II molecules with a matched HLA from which the source of T cells has been obtained. In particular embodiments, both the APCs and T cells have been isolated from the same subject, i.e., are autologous to the cancer patient. In some embodiments, the method may comprise inducing autologous antigen presenting cells (APCs) of the patient to present the mutated amino acid sequence. By using autologous APCs from the patient, the methods may identify T cells that have antigenic specificity for a mutated amino acid sequence encoded by a cancer-specific mutation that is presented in the context of an MHC molecule expressed by the patient.

In some embodiments, the APCs are cells from a blood or apheresis sample from a subject, such as the patient. In some embodiments, the APCs include cells present in a peripheral blood mononuclear cell (PBMC) sample. Typically, APCs function in a PBMC culture primarily involves monocytes and B cells. In some embodiments, a population of isolated PBMCs can be used as APCs in the provided methods. PBMCs can be obtained using standard methods such as Ficoll-Paque gradient separation. In some cases, the APCs are or include B cells that are isolated from the blood or apheresis sample or from a PBMC sample. In other cases, the APCs are or include monocytes isolated from the blood or apheresis sample or from a PBMC sample. In some aspects, the monocytes can be used as a source for preparing monocyte-derived DCs for use as APCs. In some embodiments, a source of monocyte-derived DCs (e.g. CD11c^{high}MHCII^{high}CD14^{low} cells) can be generated ex vivo from isolated monocytes, by culture with GM-CSF and IL-4 for 4 to 6 days to produce monocyte-derived dendritic cells. In particular embodiments, the monocytes are isolated from PBMCs such as by CD14 selection, and then are cultured with GM-CSF and IL-4 for 4 to 6 days.

In some embodiments, the APCs are primary cells (e.g. B cells or monocyte-derived DCs) that are replication competent, for example, the cells are not subjected to irradiation, heat treatment or other method that would result in their inactivation. In particular embodiments, the provided methods do not use irradiated APCs. In some embodiments, the APCs are freshly isolated primary cells obtained from the subject, or are derived from primary cells obtained from the subject. In some embodiments, the APCs have been cryopreserved and subsequently thawed prior to the co-culture with the stimulated T cells in accord with provided methods.

In some embodiments, the APCs are from cells from a blood sample from a subject. In some embodiments, the sample is a whole blood sample. In some embodiments, the sample is an apheresis sample. In some particular embodiments, B cells are used as a source of APCs and are generated from a patient apheresis, such as an apheresis autologous to the subject from which the tumor fragments and/or T cells were obtained. In some embodiments, the B cells are expanded from a sample from the subject ex vivo. In some embodiments, the B cells are cultured for expansion with additives that promote expansion. In some embodiments, the B cell culture includes the addition of IL-21, which in some aspects can improve cell yield and/or viability of the expanded T cells. In some embodiments, the addition of IL-21 can shorten the time period for expansion and improve the yield and/or viability of ex vivo expanded B cells.

In other particular embodiments, monocyte-derived dendritic cells are used as a source of APCs and are generated from monocytes from a patient apheresis, such as an apheresis autologous to the subject from which the tumor fragment and/or T cells are obtained.

In some embodiments, the isolated or generated APCs are collected and are cryofrozen. The provision of an intermediate hold step by cryopreservation after the isolation or generation of APCs can be used to coordinate timing with the neoepitope identification and peptide generation such as described in Section I. A and/or initial expansion of T cells, such as described in Section I.B. In some embodiments, for cryopreservation, the isolated or generated APCs are formulated as a composition with a cryoprotectant. In some embodiments, the cryoprotectant is or comprises DMSO and/or s glycerol. In some embodiments, compositions formulated for cryopreservation can be stored at low temperatures, such as ultra low temperatures, for example, storage with temperature ranges from −40° C. to −150°° C., such as or about 80° C.±6.0° C.

In some embodiments, the cryopreserved cells are prepared for subsequent steps by thawing. In some cases, the cells can be ready for subsequent culturing with T cells and peptides immediately after thawing following one or more wash steps.

In some embodiments, the APCs (e.g. PBMCs, B cells or monocyte-derived DCs) are contacted with a pool of peptides. The pool of peptide can represent many different mutated amino acid sequences, such as 5, 10, 20, 25, 30, 40, 50, 60, 70, 80, 90, 100, 150, 200, 300, 400, 500, 600, 700, 800, 900 or 100 peptides, or any value between any of the foregoing.

The peptides or pool of peptides are loaded onto antigen presenting cells (e.g. dendritic cells or B cells), such as by peptide pulsing, at a concentrations suitable for their presentation on the surface of a major histocompatibility complex (MHC).

In some embodiments, the peptide concentration representing an individual or single peptide in the pool of peptide is in a range between at or about 0.1 μg/mL and at or about 1 μg/mL, or at or about 1 μg/mL and at or about 10 μg/mL. In some embodiments, the concentration representing an individual or single peptide can be at or about 0.1 μg/mL, at or about 0.25 μg/mL, at or about 0.5 μg/mL, at or about 1 μg/mL, at or about 2.5 μg/mL, at or about 5 μg/mL, at or about 10 μg/mL, or any value between any of the foregoing.

In an embodiment, inducing APCs (e.g. B cells or monocyte-derived DCs) to present the mutated amino acid sequence comprises introducing a nucleotide sequence encoding the mutated amino acid sequence into the APCs. The nucleotide sequence is introduced into the APCs so that the APCs express and display the mutated amino acid sequence, bound to an MHC molecule, on the cell membrane. The nucleotide sequence encoding the mutated amino acid may be RNA or DNA. Introducing a nucleotide sequence into APCs may be carried out in any of a variety of different ways. Non-limiting examples of techniques that are useful for introducing a nucleotide sequence into APCs include transformation, transduction, transfection, and electroporation. In some cases, peptides for binding MHC class II restricted molecules are presented as a gene encoding DNA of the mutation and electroporated into the antigen presenting cell. This DNA will then be in-vitro transcribed into RNA encoding peptides on the surface for recognition by CD4+ cells. In some cases, Tandem Mini Gene methods can be employed to do this for MHC class II restricted molecules, see e.g. published PCT Patent Application Number WO 2016/053338 and Parkhurst et al. (2016) Clin Cancer Res., 23:2491-505. In an embodiment in which more than one gene is identified, the method may comprise preparing more than one nucleotide sequence, each encoding a mutated amino acid sequence encoded by a different gene, and introducing each nucleotide sequence into a different population of APCs. In this regard, multiple populations of APCs, each population expressing and displaying a different mutated amino acid sequence, may be obtained. For example, in the case where tandem minigenes are used, APCs (e.g. B cells or monocyte-derived DCs) are electroporated with a mixture of DNA (plurality of DNA) encoding a different mutated amino acid sequences, which will then be in-vitro transcribed into RNA encoding peptides for surface recognition by CD4+ T cells. In some embodiments, APCs (e.g. B cells or monocyte-derived DCs) are electroporated using the Lonza 4D Nucleofector continuous electroporation system.

The methods include adding T cells (e.g. from patient having a tumor) with the culture of APCs presenting the peptides and co-culturing the APCs and T cells for a period of time to allow presentation and recognition of the peptide on the surface of APCs by one or more T cells in the population. In provided embodiments, the T cells include a population of the stimulated T cells.

In some embodiments, for APCs are first contacted or incubated with peptides to prepare peptide-presenting APCs (also referred to as “loading” or “pulsing” with peptides). In some embodiments, APCs (e.g. B cells or monocyte-derived DCs) are incubated with peptides for between at or about 2 hours and at or about 48 hours, such as between at or about 2 hours and at or about 36 hours, between at or about 2 hours and at or about 24 hours, between at or about 2 hours and at or about 24 hours, between at or about 2 hours and at or about 18 hours, between at or about 2 hours and at or about 12 hours, between at or about 2 hours and at or about 6 hours, between at or about 6 hours and at or about 48 hours, between at or about 6 hours and at or about 36 hours, between at or about 6 hours and at or about 24 hours, between at or about 6 hours and at or about 24 hours, between at or about 6 hours and at or about 18 hours, between at or about 6 hours and at or about 12 hours, between at or about 12 hours and at or about 48 hours, between at or about 12 hours and at or about 36 hours, between at or about 12 hours and at or about 24 hours, between at or about 12 hours and at or about 18 hours, between at or about 18 hours and at or about 48 hours, between at or about 18 hours and at or about 36 hours, between at or about 18 hours and at or about 24 hours, between at or about 24 hours and at or about 48 hours, between at or about 24 hours and at or about 36 hours, or between at or about 36 hours and at or about 48 hours. In some embodiments, the APCs (e.g. B cells or monocyte-derived DCs) are incubated with peptides for at or about 4 hours, at or about 6 hours, at or about 7 hours, at or about 8 hours, at or about 9 hours, at or about 10 hours, at or about 12 hours, at or about 14 hours, at or about 16 hours, at or about 18 hours, at or about 20 hours, at or about 22 hours, at or about 24 hours, or any value between any of the foregoing. In some embodiments, the APCs (e.g. PBMCs, B cells or monocyte-derived DCs) are incubated with peptides overnight, such as for between at or about 8 to 12 hours. In some embodiments, the co-culture incubation is for at or about 6 hours.

In some embodiments, the T cells (e.g. stimulated T cells) and APCs (e.g. B cells or monocyte-derived DCs) can be present in a culture at a ratio of T cells to APC of 1:100 to 100:1, such as 1:50 to 50:1, 1:25 to 25:1, 1:10 to 10:1, or 1:5 to 5:1. In some embodiments, the ratio of T cells (e.g. stimulated T cells) to APC is at or about 1:100, at or about 1:50, at or about 1:25, at or about 1:10, at or about 1:5, at or about 1:2.5, at or about 1:1, at or about 2:5:1, at or about 5:1, at or about 10:1, at or about 25:1, at or about 50:1 or at or about 100:1, or any value between any of the foregoing. In some embodiments, the ratio of T cells (e.g. stimulated T cells) to APC is between 20:1 and 1:1, between 15:1 and 1:1, between 10:1 and 1:1, between 5:1 and 1:1, or between 2.5:1 and 1:1. In some embodiments, the ratio of T cells (e.g. stimulated T cells) to APC is between 1:20 and 1:1, between 1:15 and 1:1, between 1:10 and 1:1, between 1:5 and 1:1, or between 1:2.5 and 1:1. In particular embodiments, coculture will be performed by mixing the T cells, e.g. population of stimulated T cells, and APC (e.g. B cells or monocyte-derived DC) at approximately a 3:1 ratio. In some embodiments, coculture will be performed by mixing the T cells, e.g. population of stimulated T cells, and APC (e.g. B cells or monocyte-derived DC) at approximately a 1:1 ratio.

In some embodiments, recombinant IL-2 is present in the cell culture medium. In some embodiments, recombinant IL-2 is added at a concentration of 100 IU/mL to 1000 IU/mL. In some embodiments, recombinant IL-2 is added to the culture medium at a concentration between at or about 10 IU/mL and at or about 600 IU/mL, such as between at or about 10 IU/mL and at or about 400 IU/mL, between at or about 10 IU/mL and at or about 200 IU/mL, between at or about 10 IU/mL and at or about 100 IU/mL, between at or about 10 IU/mL and at or about 50 IU/mL, between at or about 50 IU/mL and at or about 400 IU/mL, between at or about 50 IU/mL and at or about 200 IU/mL, between at or about 50 IU/mL and at or about 100 IU/mL, between at or about 100 IU/mL and at or about 400 IU/mL, between at or about 100 IU/mL and at or about 200 IU/mL, between at or about 200 IU/mL and at or about 400 IU/mL, between at or about 400 IU/mL and at or about 600 IU/m. In some embodiments, recombinant IL-2 is present in an amount that is at or about 300 IU/mL.

In some embodiments, the co-culture of APCs and T cells can be incubated at a temperature suitable for the presentation of peptides on MHC and the activation of T cells in the culture, for example, at least about 25 degrees Celsius, generally at least about 30 degrees, and generally at or about 37 degrees Celsius.

In some embodiments, the co-culture incubation of peptide-presenting APCs and T cells is carried out for up to 96 hours. The co-culture incubation of peptide-presenting APC can be carried out for 12 hours to 96 hours, such as at or about 12 hours, at or about 16 hours, at or about 20 hours, at or about 24 hours, at or about 36 hours, at or about 48 hours, at or about 60 hours, at or about 72 hours, at or about 84 hours or at or about 96 hours, or for a time between any of the foregoing. In some embodiments, the co-culture is incubated for 12 to 48 hours. In some embodiments, the co-culture is incubated for 24 to 48 hours. In particular embodiments, the co-culture is incubated for 20 to 24 hours. In some embodiments, the co-culture is incubated for about 20 hours. In some embodiments, the co-culture is incubated for about 24 hours.

In some embodiments, the methods include co-culturing the second population of T cells for about 12 to 48 hours with autologous antigen presenting cells (APCs) with recombinant IL-2added at a concentration of 100 IU/mL to 1000 IU/mL to produce a third population of T cells, wherein the APCs are loaded with a pool of peptide neoantigens from the tumor, wherein each peptide is 13-40 amino acids in length and is loaded at a concentration of 100 ng/ml per peptide, and wherein the ratio of the second population of T cells to APCs is about 2:1 to 10:1. In some embodiments, the recombinant IL-2 is added at about 300 IU/mL. In some embodiments, the co-culture of T cells and APCs is at a ratio of about 5:1 T cells to APCs and is carried out for about 20 hours with IL-2 added at about 300 IU/mL. In some embodiments, the co-culture of T cells and APCs is at a ratio of about 5:1 T cells to APCs and is carried out for about 24 hours with recombinant IL-2 added at about 300 IU/mL. In some embodiments, the pool of peptide neoantigens includes up to 200 peptides, such as at least 100 peptides, 110 peptides, 120 peptides, 130 peptides, 140 peptides, 150 peptides, 160peptides, 170 peptides, 180 peptides, 190 peptides or 200 peptides, or any value between any of the foregoing. In some embodiments, the pool of peptides includes about 190 peptides.

In some embodiments, at the end of the co-culturing tumor reactive T cells are separated from APCs present in the co-culture. In some embodiments, the separation can include methods that select away or remove the APCs. In some embodiments, the separation can include methods that positively select or retain the T cells present in the co-culture. In some embodiments, total T cells in the co-culture can be selected. In particular embodiments, tumor reactive T cells or T cells that express one or more upregulation marker, e.g. activation markers, associated with tumor-reactive T cells can be selected. Exemplary methods for selection of tumor reactive T cells are described in Section II.D.

### D. Selection for Tumor Reactive T Cells

In embodiments of the provided methods, prior to the further expansion of T cells from the co-culture, the methods involve enrichment or selection of tumor reactive T cells that are surface positive for CD134 (OX40) and/or CD137 (41BB). The selected cells surface positive for CD134and/or CD137 are a fourth population of cells in accord with provided methods. The selection of the cells may be through antibody binding for CD134 and/or CD137 markers and subsequent enrichment by flow cytometry, including by methods involving magnetic separation or fluorescence-activated cell sorting (FACS). In particular embodiments, the methods involve selecting cells from the co-culture (third population of cells) for cells that are surface positive for CD134 and CD137 to produce a fourth population of cells. In some embodiments, T cells selected from the co-culture results in a population of T cells enriched for CD3+ T cells, or CD4+ cells and CD8+ cells, that are positive for one of more of such T cell activation marker CD134 and/or CD137, such as CD134 and CD137. In some embodiments, such cells include or are enriched for tumor-reactive T cells. For example, such CD3+ T cells, or CD4+ and/or CD8+ populations, can be further sorted into sub-populations by the positive selection for CD134 and/or CD137 markers expressed or expressed to a relatively higher degree on tumor-reactive T cells. In particular embodiments, the selection of cells from the co-culture produces a fourth population of cells that is an enriched population of cells for further culture under conditions for expansion, such as described in Section ILE.

Methods of isolating, selecting and/or enriching for cells can be by any of a variety of methods, such as by positive or negative selection based methods. In some embodiments, methods can include immunoaffinity-based selections. In some embodiments, the T cells can be enriched or sorted a variety of ways including, but not limited to, magnetic bead separation, fluorescent cell sorting, and disposable closed cartridge based cell sorters. In particular aspects, CD134 and/or CD137 can be used to select reactive cells using, but not limited to, florescent antibodies, nanoparticles or beads on cell selection equipment, but not limited to, the CliniMACS, Sony FX500 or the Tyto cell sorting systems (Miltenyi).

In some embodiments, the T cells can be enriched or sorted a variety of ways including, but not limited to, magnetic bead separation, fluorescent cell sorting, and disposable closed cartridge based cell sorters. In particular aspects, one or more reagents specific to CD134 and/or CD137 can be used including, but not limited to, florescent antibodies, nanoparticles or beads on cell selection equipment, but not limited to, the CliniMACS, Sony FX500 or the Tyto cell sorting systems (Miltenyi).

In certain embodiments, the sample is contacted with a binding agent, e.g., a detectably labeled binding agent, that specifically binds to the cell surface marker CD134 and/or CD137. In certain embodiments, the detectably labeled binding agent(s) are fluorescently labeled. In certain embodiments, T cells labeled with binding agents specific to the cell surface marker are identified by flow cytometry. In certain embodiments, the method further includes separating any resultant T cells labeled with the binding agent(s) from other components of the sample to produce a composition enriched for T cells surface positive for the one or more cell surface marker. Cell selection sorting equipment can be used that has a sufficiently high-throughput to handle large volumes and cell numbers. Non-limiting cell sorting equipment includes, for example, Sony FX500 or the Tyto cell sorting systems (Miltenyi).

The incubation generally is carried out under conditions whereby the antibodies or binding partners, or molecules, such as secondary antibodies or other reagents, which specifically bind to such antibodies or binding partners, which are attached to the magnetic particle or bead and/or are detectably labeled, specifically bind to cell surface molecules if present on cells within the sample. In some aspects, cells bound to the antibodies can be recovered or separated from non-bound cells in the sample.

In some aspects, the separation is carried out as automated separation of cells in a closed and sterile system. For example, components of such an automated system may include an integrated microcomputer, fluorescent light source and separation unit, peristaltic pump, and various pinch valves. In some embodiments, the integrated computer controls all components of the instrument and directs the system to perform repeated procedures in a standardized sequence.

In some embodiments, the antibodies that bind to the cell surface marker CD134 and CD137 are each coupled to a magnetic bead. In such embodiments, after incubation of cells with the magnetically labeled beads, the magnetic beads are removed or separated. In some embodiments, the magnetic separation unit includes a permanent magnet that is moveable and a holder for a selection column. In some aspects, the separation is carried out using CliniMACS system (Miltenyi Biotic) or any similar system known in the art. In certain embodiments, separation is carried out using a system equipped with a cell processing unit that permits automated washing and fractionation of cells by centrifugation. In some embodiments, the automated separation, such as using the CliniMACS system, uses antibody-coupled particles that are supplied in a sterile solution. In some embodiments, cells are labeled with detectable particles and then the cells are washed to remove excess particles. After initiation of the separation program by the computer, the system automatically applies the cell sample onto the separation column. Antibody-labelled cells are retained within the column, while unlabeled cells are removed by a series of washing steps. In some embodiments, the cell populations for use with the methods described herein are labeled and are retained in the column, and eluted from the column after removal of the magnetic field, and are collected within a cell collection bag.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system.

In particular embodiments, selection of cells is carried out by flow cytometry-based cell sorting. Compared to other methods, flow cytometry-based cell sorting has the advantage that cells can be isolated in a single step on the basis of multiple parameters for each cell, thereby achieving a higher yield of cells and a higher purity that may not be possible with bead-based (e.g. magnetic bead-based) separations. Further, multiparameter cell staining and separation allows simultaneous labeling and identification and sorting of a plurality of antigens and characteristic fluorescent signals. Using flow cytometry sorting, a single process can remove and isolate specific populations based on a complex cell surface phenotype. Cell selection sorting equipment can be used that has a sufficiently high-throughput to handle large volumes and cell numbers. Non-limiting cell sorting equipment includes, for example, Sony FX500 or the Tyto cell sorting systems (Miltenyi). For use in provided methods, the flow cytometer instrument is GMP compliant. Method of cell sorting to achieve multiparameter sorting for two or more cell surface markers (e.g. CD134 and CD137) can be carried out using multicolor fluorophore reagents that are compatible. It is within the level of a skilled artisan to choose appropriate fluorophores and reagents, such as by choosing a bright fluorophore and choosing fluorophores that have minimal to no spectral overlap. In some embodiments, the one or more fluorescent markers each individually comprise a fluorophore selected from the group consisting of PE-Cy7, APC, AF700, BV421, Aqua, and BV605.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. In some embodiments, a cell population described herein is collected and enriched (or depleted) via flow cytometry, in which cells stained for multiple cell surface markers (e.g., with a antibody-coupled fluorescent peptide) are carried in a fluidic stream.

In some embodiments, the cell staining involves incubation with an antibody or binding partner that specifically binds to such markers as described, which in some embodiments is followed by washing steps and separation of cells having bound the antibody or binding partner, from those cells having not bound to the antibody or binding partner. In some aspects of such processes, a volume of cells is mixed with an amount of a desired staining reagent and incubated under conditions for staining of the cells. In some embodiments, the staining or labelling is carried out at a temperature between 0° C. and 25° C., such as at or about 4° C. In some embodiments, the staining or labelling is carried out for greater than 5 minutes, typically greater than 15 minutes. In some embodiments, the staining or labelling is carried out for between 15 minutes and 6 hours, such as between 30 minutes and 2 hours. In some embodiments, the staining or labelling is carried out for example, at or about 15 minutes, 30 minutes, 1 hour, 1.5 hours, 2 hours, 2.5 hours, 3 hours, or any value between any of the foregoing. In some embodiments, the labeling with the one or more staining reagents is carried out simultaneously. In some embodiments, one or more wash steps are carried out prior to introducing the sample into the flow cytometer for analysis.

In some embodiments, the cell sample is prepared by suspending single cells at a density of 1×10⁶ to 5×10⁷ cells/ml in order to allow the cells to pass through the flow cytometer for reading. In some embodiments, the density of cells for sorting is 5×10⁶ cells/mL to 50×10⁶ cells/mL, such as at or about 20×10⁶ cells/mL. In some embodiments, this concentration of cells is called the fluid sheath. In some embodiments, the fluid sheath influences the rate of flow sorting, which typically progresses at around 2,000-20,000 cells (events) per second. The cell sample's) fluid sheath is typically made of a phosphate buffered saline solution, but other solutions are available as will be known and understood by those skilled in the art.

In some embodiments, the flow cytometry sorting rate In some embodiments, the flow cytometry sorting rate is from 2000 events/second to 10,000 event/second. In some embodiments, the flow cytometry sorting rate is about 2000 events/second, about 3000 events/second, about 4,000 events/second, about 5000 events/second, about 6000 events/second, about 7000 events/second, about 8000 events/second, about 9000 events/second, about 10000 events/second, about 15000 events/second, or about 20000 events per second, or any value between any of the foregoing. In some embodiments, the flow cytometry sorting rate is about 6,000 events/second.

In some embodiments, the sample is introduced into a flow cytometer. The cell sample is typically narrowed down to a single stream through a fluidics system with the application of hydro pressure. This stream is then passed through the one or more beams of light scattering or fluorescence emission. Lasers typically serves as the light source in flow cytometers. The laser produces a single wavelength of light that once contacted with the cell sample produces scattered light in the forward direction as a measure of cell size, scattered light in the side direction as a measure of cell complexity, and fluorescent light, also emitted in the side direction which is proportional to the relative amount of a particular cell marker. Fluorescent channels are usually indicated by the designations FL1, FL2, FL3, etc., depending on the number of channels in the instrument. Each fluorescent channel is set with barrier filters to detect a selected specific dye while filtering out all others. The channel in which a specific dye is predominantly detectable may be referred to as its primary fluorescent channel while other fluorescent channels may be designated as secondary channels. Scattered and fluorescent emitted light signals are converted to electronic pulses that are processed by the flow cytometry engine and displayed on a graphical user interface “GUI.”

Methods of analyzing flowcytometric or FACS data can involve a “gating” for data to separate specific groups of cells. Different cell types can be identified by the scatter parameters and the fluorescence emissions resulting from labeling various cell proteins with dye-labeled antibodies as described above. The identification of clusters and, thereby, populations can be carried out by “gating” of the cells. In some embodiments, gates corresponding to subsets of particles of interest, such as TIL expressing markers of neoantigen reactivity, are defined by users with the aid of software operationally associated with the flow system as described above.

In some embodiments, a gate may be a “threshold” gate, which is a gate for only one optical parameter that defines an open region within the multidimensional space. In some embodiments, “threshold” gating can been used for forward light scatter to remove high frequency low level signals caused by interference, such as debris in the sample. In some embodiments, “window” gating is employed, e.g., by defining upper and lower bounds for signal values. In some embodiments, gating is carried out on a 2D-plot of two parameters, such as side scatter (e.g., on vertical axis) and a fluorescence signal (e.g., on horizontal axis).

In some embodiments, flow cytometry for a cell surface marker includes gating for an “F-minus one” (FMO) control. FMO gating includes separate portions of the same sample stained with a panel of detectably labeled binding agents that contains all the agents but one. The distribution of the signal of the removed fluorophore can be used to define the positive threshold for the missing label as it is known that all cells are negative in the control. The position of all gates can be determined using fluorescence minus one (FMO) controls in which the antibody against the investigated marker is substituted with an appropriate isotype control. In some embodiments, a gate can be drawn using cells stained with the FMO cocktail around cells positive for CCD137 and/or CD134. In some embodiments, staining with other labeled antibody reagents or scatter analysis can be carried out sequential gating to exclude other cell populations (e.g. APCs) and/or to enrich for lymphocytes (e.g. CD3+ or CD4+ and/or CD8+ cells). In some embodiments, a viability dye also can be added. An exemplary viability dye is 7-ADD. In some embodiments, a gate can be drawn around cells negative for 7-AAD (7-AADmeg).

In some embodiments, the cells are sorted into a single population of cells and collected. The selected population of cells is referred to as the fourth population of cells and is used as input for expansion, such as described in Section II.E.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker.

In some embodiments, the selections produces an enriched population of cells, such as a population of cells enriched for CD3+ T cells or CD4+ cells and CD8+ cells, that are further positive for CD134 and/or CD137. In some embodiments, such cells include or are enriched for tumor-reactive T cells or T cells associated with tumor-reactive T cells. In some embodiments, the enriched population of cells is used in subsequent processing steps, such as subsequent processing steps involving incubation, stimulation or activation, and/or expansion in accord with one or more steps of any of the provided methods.

In some embodiments, the enriched population of cells are enriched cells from a starting sample as describe above, in which the percentage of cells of a particular phenotype, e.g. tumor-reactive CD3+ T cells or CD3+ T cells surface positive for CD134 and/or CD137, in the enriched population of cells in increased by at least 10%, 20%, 30%, 40%, 50%, 60%, 70%, 80%, 90%, 100%, 500%, 1000%, 5000% or more greater than the percentage of such cells in the starting sample. In some embodiments, the purity of tumor-reactive CD3+ T cells or CD3+ T cells surface positive for CD134 and/or CD137 in the enriched composition, i.e. the percentage of cells positive for the selected cell surface marker versus total cells in the population of enriched cells, is at least 90%, 91%, 92%, 93%, 94%, and is generally at least 95%, 96%, 97%, 98%, 99% or greater.

### E. Further Expansion and Harvesting

In some embodiments, the selected T cells from the co-culture (e.g. fourth population of cells) are further incubated under conditions to expand the cells ex vivo following the co-culture. The incubation is carried out in the presence of one or more T cell stimulatory agent(s) under conditions for stimulating the T cells, such as to expand the T cells. In some embodiments, the incubation is a rapid expansion protocol (REP).

In some of any of the provided embodiments, the T cell stimulatory agent(s) is selected from an agent that initiates TCR/CD3 intracellular signaling and/or an agent that initiates signaling via a costimulatory receptor. An anti-CD3 antibody can include any antibody directed against or that can specifically bind the CD3 receptor on the surface of T cells, typically human CD3 on human T cells. Anti-CD3 antibodies include OKT3, also known as muromonab. Anti-CD3 antibodies also include the UHCTI clone, also known as T3 and CD3E. Other anti-CD3 antibodies include, for example, otelixizumab, teplizumab, and visilizumab. The anti-CD3 antibody can be added as a soluble reagent or bound to a bead. In particular embodiments, the anti-CD3 antibody is soluble. In some of any of the provided embodiments, the agent that initiates TCR/CD3 intracellular signaling is an anti-CD3 antibody, such as OKT3.

In particular embodiments, the T cell stimulatory agent(s) include an anti-CD3 antibody, which is added to the cell culture medium during the incubation. In some embodiments, the anti-CD3 antibody is added at a concentration ranging between at or about 0.1 ng/ml and 50 ng/ml, such between at or about 0.5 ng/ml and at or about 50 ng/ml, between at or about 0.5 ng/mL and at or about 30 ng/ml, between at or about 0.5 ng/ml and at or about 15 ng/ml, between at or about 0.5 ng/ml and at or about 5 ng/ml, between at or about 0.5 ng/ml and at or about 1 ng/ml, between at or about 1 ng/mL and at or about 50 ng/ml, between at or about 1 ng/ml and at or about 30 ng/ml, between at or about 1 ng/ml and at or about 15 ng/ml, between at or about 1 ng/ml and at or about 5 ng/mL, between at or about 5 ng/mL and at or about 50 ng/mL, between at or about 5 ng/ml and at or about 30 ng/mL, between at or about 5 ng/mL and at or about 15 ng/ml, between at or about 15 ng/ml and at or 50 ng/mL, between at or about 15 ng/mL and at or about 30 ng/mL or between at or about 30 ng/mL and at or about 50 ng/mL, each inclusive. In an embodiment, the anti-CD 3 is added to the cell culture medium at about 0.1 ng/mL, about 0.5 ng/ml, about 1 ng/ml, about 2.5 ng/mL, about 5 ng/ml, about 7.5 ng/ml, about 10 ng/mL, about 15 ng/mL, about 20 ng/mL, about 25 ng/mL, about 30 ng/mL, about 35 ng/mL, about 40 ng/ml, about 50 ng/ml. In some embodiments, the anti-CD3 antibody is added at a concentration of about 30 ng/ml. In particular embodiments, the anti-CD3 antibody is OKT3.

In some embodiments, the T cell stimulatory agent can include adding the anti-CD3 antibody and additionally include adding to the population of T cells feeder cells, such as non-dividing peripheral blood mononuclear cells (PBMC). In some aspects, the PBMCs provide a CD28-mediated signal to provide a costimulatory signal to the T cells. In some aspects, the non-dividing feeder cells can be irradiated PBMC feeder cells. In some embodiments, the PBMC are irradiated with gamma rays in the range of about 3000 to 3600 rads to prevent cell division. In some embodiments, the non-dividing PBMCs, such as irradiated PBMCs, are added at a ratio of PBMCs to T cells that is between 100:1 to 500:1. In some embodiments, the non-dividing PBMCs, such as irradiated PBMCs, are added at a ratio of PBMCs to T cells that is 100:1, 150:1, 200:1, 250:1, 300:1, 350:1, 400:1, 450:1 or 500:1, or any value between any of the foregoing. In some embodiments, the non-dividing PBMCs, such as irradiated PBMCs, are added at a ratio of PBMCs to T cells that is about 200:1.

In general, the culturing and incubations can occur in the presence of recombinant IL-2. In some embodiments, recombinant IL-2 s added or is exogenous to the culture media. In some embodiments, the recombinant IL-2 is added to the culture medium at a concentration between at or about 1000 IU/mL at or about 8000 IU/mL, such as between at or about 1000 IU/mL and at or about 7000 IU/mL, between at or about 1000 IU/mL and at or about 6000 IU/mL, between at or about 1000 IU/mL and at or about 5000 IU/mL, between at or about 1000 IU/mL and at or about 4000 IU/mL, between at or about 1000 IU/mL and at or about 2000 IU/mL, 2000 IU/mL at or about 8000 IU/mL, between at or about 2000 IU/mL and at or about 7000 IU/mL, between at or about 2000 IU/mL and at or about 6000 IU/mL, between at or about 2000 IU/mL and at or about 5000 IU/mL, between at or about 2000 IU/mL and at or about 4000 IU/mL, 4000 IU/mL at or about 8000 JU/mL, between at or about 4000 IU/mL and at or about 7000 IU/mL, between at or about 4000 IU/mL and at or about 6000 IU/mL, between at or about 4000 IU/mL and at or about 5000 IU/mL, between at or about 5000 IU/mL at or about 8000 IU/mL, between at or about 5000 IU/mL and at or about 7000 IU/mL, between at or about 5000 IU/mL and at or about 6000 IU/mL, between at or about 6000 IU/mL at or about 8000 IU/mL, between at or about 6000 IU/mL and at or about 7000 IU/mL or between at or about 7000 IU/mL and at or about 8000 IU/mL. In some embodiments, recombinant IL-2 is present in an amount that is or is about 6000 IU/mL.

In some embodiments, the selected cells from the co-culture (e.g. fourth population of cells) are expanded by culture of the T cells with irradiated PBMCs added at a ratio of 200:1 iPBMC to TIL, with 6000 IU/mL human recombinant IL-2 and 30 ng/mL anti-CD3 antibody (OKT3).

The sorted or selected T cells can be expanded under the one or more stimulatory conditions in a culture vessel suitable for cell expansion. In some embodiments, the culture vessel is a gas permeable culture vessel, such as a G-Rex system (e.g. G-Rex 10, G-Rex 10M, G-Rex 100 M/100M-CS or G-Rex 500 M/500 M-CS). In some embodiments the culture vessel is a microplate, flask, bar or other culture vessel suitable for expansion of cells in a closed system. In some embodiments, expansion can be carried out in a bioreactor. In some embodiments the composition of expanded T cells is removed from a closed system and placed in and/or connected to a bioreactor for expansion. The sorted or selected T cells can be expanded using a cell expansion system by transfer to the cell to gas permeable bags, such as in connection with a bioreactor (e.g. Xuri Cell Expansion System W25 (GE Healthcare)). In an embodiment, the cell expansion system includes a culture vessel, such as a bag, e.g. gas permeable cell bag, with a volume that is about 50 mL, about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L, or any value between any of the foregoing. In some embodiments, the process is automated or semi-automated. Examples of suitable bioreactors for the automated perfusion expansion include, but are not limited to, GE Xuri W25, GE Xuri W5, Sartorius BioSTAT RM 20|50, Finesse SmartRocker Bioreactor Systems, and Pall XRS Bioreactor Systems, or Miltenyi Prodigy. In some aspects, the expansion culture is carried out under static conditions. In some embodiments, the expansion culture is carried out under rocking conditions. The medium can be added in bolus or can be added on a perfusion schedule. In some embodiments, the bioreactor maintains the temperature at or near 37° C. and CO2 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the culturing is performed with perfusion, such as with a rate of 290 ml/day, 580 ml/day, and/or 1160 ml/day.

In some embodiments, the cells are seeded in an appropriate culture vessel (e.g. gas permeable bag) at a density of from 0.5×10⁶ cells/mL to 1.5×10⁶ cells/mL. In some embodiments, the density is at or about 0.5×10⁶ cells/mL, 0.75×10⁶ cells/mL, 1×10⁶ cells/mL, 1.25×10⁶ cells/mL or 1.5×10⁶ cells/mL, or any value between any of the foregoing.

In some aspects, cells are expanded in an automated closed expansion system that is perfusion enabled. Perfusions can continuously add media to the cells to ensure an optimal growth rate is achieved.

The expansion methods can be carried out under GMP conditions, including in a closed automated system and using serum free medium. In some embodiments, any one or more of the steps of the method can be carried out in a closed system or under GMP conditions.
some embodiments, the incubation with the T cell stimulatory agent(s) for expansion of tumor-reactive cells is carried out until a threshold number of cells is achieved. In some embodiments, the threshold number of cells is about 100-fold or more greater in number than the number of selected TILs in the fourth population of T cells prior to the expansion. In some embodiments, the threshold number of cell is a number of TILs that is greater than the number of selected TILs in the fourth population prior to the expansion by about 200-fold, 300-fold, 400-fold, 500-fold, 600-fold, 700-fold, 800-fold, 900-fold, 1000-fold, 1250-fold, 1500-fold or 2000-fold, or any value between the foregoing.

In some embodiments, the incubation with the T cell stimulatory agent(s) for expansion is for 7 to 21 days, such as 7 days, 8 days, 9 days, 10 days, 11 days, 12 days, 13 days, 14, days, 15 days, 16 days, 17 days, 18 days, 19 days, 20 days or 21 days, or any value between any of the foregoing. In some embodiments, the incubation is carried out for 7-14 days. In some embodiments, the incubation is for about 10 days. In some embodiments, the incubation is for about 11 days. In some embodiments, the incubation is for about 12 days. In some embodiments, the incubation is for about 13 days. In some embodiments, the incubation is for about 14 days. In some cases, media can be exchanged daily, every other day, every third day, every 5^{th} day or once a week during the time of the culture or incubation. In some embodiments, the media exchange is at Day 5 and Day 10. In some embodiments, the recombinant IL-2 is replenished (added) at each media exchange. In some embodiments, about 6000 IU/mL recombinant IL-2 is replenished (added) at each media exchange.

In some of any of the provided embodiments, the culturing is carried out until a threshold amount of cells is achieved that is between at or about 0.5×10⁸ and at or about 50×10⁹ total cells or total viable cells, between at or about 0.5×10⁸ and at or about 30×10⁹ total cells or total viable cells, between 0.5×10⁸ and at or about 12×10⁹ total cells or total viable cells, between at or about 0.5×10⁸ and at or about 60×10⁸total cells or total viable cells, between at or about 0.5×10⁸ and at or about 15×10⁸ total cells or total viable cells, between at or about 0.5×10⁸ and at or about 8×10⁸ total cells or total viable cells, between at or about 0.5×10⁸ and at or about 3.5×10⁸ total cells or total viable cells, between at or about 0.5×10⁸ and at or about 1×10⁸ total cells or total viable cells, between 1×10⁸ and at or about 50×10⁹ total cells or total viable cells, between at or about 1×10⁸ and at or about 30×10⁹ total cells or total viable cells, between 1×10⁸ and at or about 12×10⁹ total cells or total viable cells, between at or about 1×10⁸ and at or about 60×10⁸ total cells or total viable cells, between at or about 1×10⁸ and at or about 15×10⁸ total cells or total viable cells, between at or about 1×10⁸ and at or about 8×10⁸ total cells or total viable cells, between at or about 1×10⁸ and at or about 3.5×10⁸ total cells or total viable cells, between at or about 3.5×10⁸ and at or about 50×10⁹ total cells or total viable cells, between at or about 3.5×10⁸ and at or about 30×10⁹ total cells or total viable cells, between at or about 3.5×10⁸ and at or about 12×10⁹ total cells or total viable cells, between at or about 3.5×10⁸ and at or about 60×10⁸ total cells or total viable cells, between at or about 3.5×10⁸ and at or about 15×10⁸ total cells or total viable cells, between at or about 3.5×10⁸ and at or about 8×10⁸ total cells or total viable cells, between at or about 8×10⁸ and at or about 50×10⁹ total cells or total viable cells, between at or about 8×10⁸ and at or about 30×10⁹ total cells or total viable cells, between at or about 8×10⁸ and at or about 12×10⁹ total cells or total viable cells, between at or about 8×10⁸ and at or about 60×10⁸ total cells or total viable cells, between at or about 8×10⁸ and at or about 15×10⁸ total cells or total viable cells, between at or about 15×10⁸ and at or about 50×10⁹ total cells or total viable cells, between at or about 15×10⁸ and at or about 30×10⁹ total cells or total viable cells, between at or about 15×10⁸ and at or about 12×10⁹ total cells or total viable cells, between at or about 15×10⁸ and at or about 60×10⁸ total cells or total viable cells, between at or about 60×10⁸ and at or about 50×10⁹ total cells or total viable cells, between at or about 60×10⁸ and at or about 30×10⁹ total cells or total viable cells, between at or about 60×10⁸ and at or about 12×10⁹ total cells or total viable cells, between at or about 12×10⁹ and at or about 50×10⁹ total cells or total viable cells, between at or about 12×10⁹ and at or about 30×10⁹ total cells or total viable cells, or between at or about 30×10⁹ and at or about 60×10⁹ total cells or total viable cells, each inclusive.

In some of any of the provided embodiments, the method results in a fold-expansion of T cells that is at least at or about 100-fold, at least at or about 250-fold, at least at or about 500-fold, at least at or about 1000-fold, or more compared to the number of cells in the fourth population of cells before the expansion.

Upon reaching a therapeutic dose after expansion the product can be concentrated and frozen in cryopreservation medium. In some embodiments, the cryoprotectant is or comprises DMSO and/or s glycerol. In some embodiments, compositions formulated for cryopreservation can be stored at low temperatures, such as ultra low temperatures, for example, storage with temperature ranges from −40° C. to −150° C., such as or about 80° C.±6.0° C.

Also provided herein are populations of T cells produced by methods described herein and pharmaceutical compositions thereof. The composition of cells are enriched in tumor-reactive T cells. In some embodiments, the composition of cells is characterized by one or more features as described in Section II.

### III. METHODS OF TREATMENT AND THERAPEUTIC APPLICATION

Provided herein are compositions and methods relating to the provided therapeutic cell compositions described herein for use in treating diseases or conditions in a subject such as a cancer. Such methods and uses include therapeutic methods and uses, for example, involving administration of the therapeutic cells, or compositions containing the same, to a subject having a disease, condition, or disorder. In some cases, the disease or disorder is a tumor or cancer. In some embodiments, the cells or pharmaceutical composition thereof is administered in an effective amount to effect treatment of the disease or disorder. Uses include uses of the cells or pharmaceutical compositions thereof in such methods and treatments, and in the preparation of a medicament in order to carry out such therapeutic methods. In some embodiments, the methods thereby treat the disease or condition or disorder in the subject.

In some embodiments, the cell compositions provided herein are autologous to the subject to be treated. In such embodiments, the starting cells for expansion are isolated directly from a biological sample from the subject as described herein, in some cases including with enrichment for T cells surface positive for one or more T cell activation marker as described, and cultured under conditions for expansion as provided herein. In some aspects, the biological sample from the subject is or includes a tumor or lymph node sample and such sample tumor and an amount of such tissue is obtained, such as by resection or biopsy (e.g. core needle biopsy or fine-needle aspiration). In some embodiments, following the culturing under conditions for expansion in accord with the provided methods the cells are formulated and optionally cryopreserved for subsequent administration to the same subject for treating the cancer.

In some embodiments, the methods of treatment comprise administering an effective amount of a composition containing tumor reactive CD3+ T cells or CD3+ T cells surface, which may include T cells surface positive for one or more activation marker. Such compositions can include any as described herein, including compositions produced by the provided methods.

In some embodiment, a subject (e.g. autologous) is administered from at or about 10⁵ to at or about 10¹² CD3+ T cells produced by any of the provided methods, or from at or about 10⁵ to at or about 10⁸ CD3+ T cells produced by any of the provide methods, or from at or about 10⁶ and at or about 10¹² CD3+ T cells produced by any of the provided methods, or from at or about 10⁸ and at or about 10¹¹ CD3+ T cells produced by any of the provided methods, or from at or about 10⁹ and at or about 10¹⁰ CD3+ T cells produced by any of the provided methods. In some embodiments, the therapeutically effective amount for administration comprises greater than or greater than at or about 10⁵ CD3+ T cells produced by any of the provided methods, at or about 10⁶ CD3+ T cells produced by any of the provided methods, at or about 10⁷ CD3+ T cells produced by any of the provided methods, at or about 10⁸ CD3+ T cells produced by any of the provided methods, at or about 10⁹ CD3+ T cells produced by any of the provided methods, at or about 10¹⁰ CD3+ T cells produced by any of the provided methods, at or about 10¹¹ CD3+ T cells produced by any of the provided methods, or at or about 10¹² CD3+ T cells produced by any of the provided methods. In some embodiments, such an amount can be administered to a subject having a disease or condition, such as to a cancer patient. In some embodiments, the number of T cells are administered are viable T cells.

In some embodiments, the methods of treatment comprise administering an effective amount of a composition containing tumor reactive CD3+ T cells or CD3+ T cells surface positive for one or more activation marker. Such compositions can include any as described herein, including compositions produced by the provided methods. In some embodiment from at or about 10⁵ to at or about 10¹² tumor reactive CD3+ T cells or CD3+ T cells surface positive for one or more activation marker, such as any as described, or from at or about 10⁵ to at or about 10⁸ tumor reactive CD3+ T cells or CD3+ T cells surface positive for one or more activation marker, or from at or about 10⁶ and at or about 10¹² tumor reactive CD3+ T cells or CD3+ T cells surface positive for one or more activation marker, or from at or about 108 and at or about 10¹¹ tumor reactive CD3+ T cells or CD3+ T cells surface positive for one or more activation marker, or from at or about 109 and at or about 10¹⁰ tumor reactive CD3+ T cells or CD3+ T cells surface positive for one or more activation marker are administered to the individual. In some embodiments, the therapeutically effective amount for administration comprises greater than or greater than at or about 10⁵ tumor reactive CD3+ T cells or CD3+ T cells surface positive for one or more activation marker, at or about 10°tumor reactive CD3+ T cells or CD3+ T cells surface positive for one or more activation marker, at or about 10⁷ tumor reactive CD3+ T cells or CD3+ T cells surface positive for one or more activation marker, at or about 10⁸ tumor reactive CD3+ T cells or CD3+ T cells surface positive for one or more activation marker, at or about 10⁹ tumor reactive CD3+ T cells or CD3+ T cells surface positive for one or more activation marker, at or about 10¹⁰ tumor reactive CD3+ T cells or CD3+ T cells surface positive for one or more activation marker, at or about 10¹⁰ tumor reactive CD3+ T cells or CD3+ T cells surface positive for one or more activation marker, or at or about 10¹² tumor reactive CD3+ T cells or CD3+ T cells surface positive for one or more activation marker. In some embodiments, such an amount can be administered to a subject having a disease or condition, such as to a cancer patient. In some embodiments, the number of T cells are administered are viable T cells.

In some embodiments, the amount is administered as a flat dose. In other embodiments, the amount is administered per kilogram body weight of the subject.

In some embodiments, the composition, such as produced by any of the provided methods or containing tumor-reactive T cells or T cells surface positive for a T cell activation marker, are administered to an individual soon after expansion according to the provided methods. In other embodiments, the expanded T cells, such as expanded tumor-reactive T cells or T cells surface positive for a T cell activation marker, are cryopreserved prior to administration, such as by methods described above. For example, the T cells, such as tumor-reactive T cells or T cells surface positive for a T cell activation marker, can be stored for greater than 6, 12, 18, or 24 months prior to administration to the individual. Such cryopreserved cells can be thawed prior to the administration.

In some embodiments, the provided compositions, such as provided by any of the provided methods or containing tumor-reactive T cells or T cells surface positive for a T cell activation marker, can be administered to a subject by any convenient route including parenteral routes such as subcutaneous, intramuscular, intravenous, and/or epidural routes of administration.

In some embodiments, the compositions, such as provided by any of the provided methods or containing tumor-reactive T cells or T cells surface positive for a T cell activation marker may be administered in a single dose. Such administration may be by injection, e.g., intravenous injection. In some embodiments, tumor-reactive T cells or T cells surface positive for a T cell activation marker may be administered in multiple doses. Dosing may be once, twice, three times, four times, five times, six times, or more than six times per year. Dosing may be once a month, once every two weeks, once a week, or once every other day. Administration of such compositions and cells may continue as long as necessary.

In some embodiments, the subject is administered a lymphodepleting therapy prior to the administration of the dose of cells from a provided compositions, such as produced by any of the provided methods or containing tumor-reactive T cells or T cells surface positive for a T cell activation marker. The lymphodepleting therapy can include administration of fludarabine and/or cyclophosphamide (the active form being referred to as mafosfamide) and combinations thereof. Such methods are described in Gassner et al. (Cancer Immunol Immunother. 201 1, 60(1):75-85, Muranski, et al, Nat Clin Pract Oncol, 2006 3(12):668-681, Dudley, et al., J Clin Oncol 2008, 26:5233-5239, and Dudley, et al., J Clin Oncol. 2005, 23(10):2346-2357, all of which are incorporated by reference herein in their entireties. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day, or a dosage amount between a range of any of the foregoing. In some embodiments, the fludarabine is for 2-7 days, such as for 3-5 days, such as at or about 3 days, at or about 4 days or at or about 5 days. In some embodiments, the cyclophosphamide is administered at a dosage of 100 mg/m2/day, 150 mg/m2/day, 175 mg/m2/day, 200 mg/m2/day, 225 mg/m2/day, 250mg/m 2/day, 275 mg/m 2/day, or 300 mg/m2/day. In some embodiments, the cyclophosphamide is administered intravenously (i.e., i.v.). In some embodiments, the cyclophosphamide treatment is for 2-7 days, such as 3-5 days, at or about 3 days, at or about 4 days or at or about 5 days. The lymphodepleting therapy is administered prior to the provided cell compositions. In some embodiments, the lymphodepleting therapy is carried out within a week of the administration of the provided cell compositions, such as 5-7 days prior to the administration of the dose of cells.

The compositions described herein can be used in a method for treating hyperproliferative disorders. In a preferred embodiment, they are for use in treating cancers. In some aspects, the cancer can be a melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the cancer is a cancer with a high mutational burden. In some embodiments, the cancer is melanoma, lung squamous, lung adenocarcinoma, bladder cancer, lung small cell cancer, esophageal cancer, colorectal cancer, cervical cancer, head and neck cancer, stomach cancer or uterine cancer.

In some embodiments, the cancer is an epithelial cancer. In some embodiments, the cancer is selected from non-small cell lung cancer (NSCLC), CRC, ovarian cancer, breast cancer, esophageal cancer, gastric cancer, pancreatic cancer, cholangiocarcinoma cancer, endometrial cancer. In some embodiments, the breast cancer is HR+/Her2− breast cancer. In some embodiments, the breast cancer is a triple negative breast cancer (TNBC). In some embodiments, the breast cancer is a HER2+ breast cancer.

In some embodiments, the subject has a cancer that is a hematological tumor. Non-limiting examples of hematological tumors include leukemias, including acute leukemias (such as 1 lq23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstro″s macroglobulinemia, heavy chain disease, myelodysplastic syndrome, hairy cell leukemia and myelodysplasia.

In some embodiments, the subject has a solid tumor cancer. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilm” tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the cancer is a skin cancer. In particular embodiments, the cancer is a melanoma, such as a cutaneous melanoma. In some embodiments, the cancer is a merkel cell or metastatic cutaneous squamous cell carcinoma (CSCC).

In some embodiments, the tumor is a carcinoma, which is a cancer that develops from epithelial cells or is a cancer of epithelial origin. In some embodiments, the cancer arises from epithelial cells which include, but are not limited to, breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

In some embodiments, the subject has a cancer that is a gastrointestinal cancer involving a cancer of the gastrointestinal tract (GI tract), including cancers or the upper or lower digestive tract, or an accessory organ of digestion, such as esophagus, stomach, biliary system, pancreas, small intestine, large intestine, rectum or anus. In some embodiments, the cancer is an esophageal cancer, stomach (gastric) cancer, pancreatic cancer, liver cancer (hepatocellular carcinoma), gallbladder cancer, cancer of the mucosa-associated lymphoid tissue (MALT lymphoma), cancer of the biliary tree, colorectal cancer (including colon cancer, rectum cancer or both), anal cancer, or a gastrointestinal carcinoid tumor. In particular embodiments, the cancer is a colorectal cancer.

In some embodiments, the cancer is a colorectal cancer (CRC). Colorectal cancer (CRC) is a common tumor of increasing incidence, which, in many cases, does not response to checkpoint inhibition or other immunotherapy. This is the case even though such cancers have properties that are associated with response, e.g., a reasonably high mutation rate and well established association of prognosis with level of T cell infiltration.

In some embodiments, the cancer is an ovarian cancer. In some embodiments, the cancer is a triple-negative breast cancer (TNBC).

In some embodiments, the cancer is lung cancer. In some embodiments, the cancer is a breast cancer. In some embodiments, the cancer is a colorectal cancer. In some embodiments, the cancer is pancreatic cancer. In some embodiments, the cancer is a merkel cell cancer. In some embodiments, the cancer is a metastatic cutaneous squamous cell carcinoma (CSCC). In some embodiments, the cancer is a melanoma. In some embodiments, the cancer is a non-small cell lung cancer (NSCLC).

In some embodiments, the subject is one whose cancer is refractory to, and or who has relapsed following treatment with, a checkpoint blockade, such as an anti-PD1 or anti-PD-L1 therapy.

In some embodiments, the subject is the same subject from with the biological sample was obtained for producing the therapeutic cell composition. In some such embodiments, the provided methods of treatment is an adoptive cell therapy with a therapeutic composition containing T cells autologous to the subject.

In some embodiments, the cell compositions provided herein are allogenic to the subject to be treated. In some aspects, the subject from which the cells are derived or isolated is a healthy subject or is not known to have a disease or conditions, such as a cancer. In such embodiments, the starting cells for expansion are isolated directly from a biological sample from such a subject as described herein, in some cases including with enrichment for T cells surface positive for one or more T cell activation marker as described, and cultured under conditions for expansion as provided herein.

In some aspects, the biological sample from the subject is or includes a tumor or lymph node sample and such sample tumor and an amount of such tissue is obtained, such as by resection or biopsy (e.g. core needle biopsy or fine-needle aspiration). In some embodiments, following the culturing under conditions for expansion the cells are formulated and optionally cryopreserved for subsequent administration to a different subject for treating a cancer in such different subject.

In some embodiments, the provided methods can be carried out with one or more other immunotherapies. In some embodiments, the immunotherapy is an immune modulating agent that is an immune checkpoint inhibitor. In some embodiments, the immune checkpoint inhibitor specifically binds a molecule selected from among CD25, PD-1, PD-L1, PD-L2, CTLA-4, LAG-3, TIM-3, CD137 (4-1BB), GITR, CD40, CD40L, CD134 (OX40), OX40L, CXCR2, B7-H3, B7-H4, BTLA, HVEM, CD28, TIGIT and VISTA. In some embodiments, the immune checkpoint inhibitor is and antibody or antigen-binding fragment, a small molecule or a polypeptide. In some embodiments, the immune checkpoint inhibitor is selected from among nivolumab, pembrolizumab, pidilizumab, MK-3475, BMS-936559, MPDL3280A, ipilimumab, tremelimumab, IMP31, BMS-986016, urelumab, TRX518, dacetuzumab, lucatumumab, SEQ-CD40, CP-870, CP-893, MED16469, MEDI4736, MOXR0916, AMP-224, and MSB001078C, or is an antigen-binding fragment thereof.

In some embodiments, the provided methods include combination therapy of a cell therapy as described and PD-1 or PD-L1 inhibitors. A PD-1 or PD-L1 inhibitor can include binding antibodies, antagonists, or inhibitors (i.e., blockers).

In an embodiment, the PD-I inhibitor is nivolumab (commercially available as OPDIVO from Bristol-Myers Squibb Co.), or biosimilars, antigen-binding fragments, conjugates, or variants thereof. Nivolumab is a fully human IgG4 antibody blocking the PD-I receptor. In an embodiment, the anti-PD-I antibody is an immunoglobulin G4 kappa, anti (human CD274) antibody. Nivolumab is assigned Chemical Abstracts Service (CAS) registry number 946414-94-4 and is also known as 5C4, BMS-936558, AtIDX-1106, and ONO-4538. The preparation and properties of nivolumab are described in U.S. Pat. No. 8,008,449 and International Patent Publication No. WO 2006/121168.

In another embodiment, the PD-1 inhibitor comprises pembrolizumab (commercially available as KEYTRUDA from Merck & Co., Inc., Kenilworth, NJ, USA), or antigen-binding fragments, conjugates, or variants thereof. Pembrolizumab is assigned CAS registry number 1374853-91-4 and is also known as lambrolizumab, MK-3475, and SCH-900475. The properties, uses, and preparation of pembrolizumab are described in International Patent Publication No. WO 2008/156712 Al, U.S. Pat. No. 8,354,509 and U.S. Patent Application Publication Nos. US 2010/0266617 AI, US 2013/0108651 A1, and US 2013/0109843 A2.

In an embodiment, the PD-LI inhibitor is durvalumab, also known as MEDI4736 (which is commercially available from Medimmune, LLC, Gaithersburg, JVIaryland, a subsidiary of AstraZeneca plc.), or antigen-binding fragments, conjugates, or variants thereof. In an embodiment, the PD-LI inhibitor is an antibody disclosed in U.S. Pat. No. 8,779,108 or U.S. Patent Application Publication No. 2013/0034559.

In an embodiment, the PD-LI inhibitor is avelumab, also known as MSB0010718C (commercially available from Merck kGaA/EMD Serono), or antigen-binding fragments, conjugates, or variants thereof. The preparation and properties of avelumab are described in U.S. Patent Application Publication No. US 2014/0341917 A1.

In an embodiment, the PD-LI inhibitor is atezolizumab, also known as MPDL3280A or RG7446 (commercially available as TECENTRIQ from Genentech, Inc., a subsidiary of Roche Holding AG, Basel, Switzerland), or antigen-binding fragments, conjugates, or variants thereof. In an embodiment, the PD-LI inhibitor is an antibody disclosed in U.S. Pat. No. 8,217,149, the disclosure of which is specifically incorporated by reference herein. In an embodiment, the PD-LI inhibitor is an antibody disclosed in U.S. Patent Application Publication Nos. 2010/0203056 A1, 2013/0045200 A1, 2013/0045201 A1, 2013/0045202 A1, or 2014/0065135 A1. The preparation and properties of atezolizumab are described in U.S. Pat. No. 8,217,149.

### IV. KITS AND ARTICLES OF MANUFACTURE

Provided herein are articles of manufacture and kits comprising the provided compositions, such as compositions containing T cells produced by any of the provided methods or containing or enriched for tumor-reactive T cells. In some embodiments, the compositions are produced by any of the provided methods.

Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. In some embodiments, the kits can further contain reagents for collection of samples, preparation and processing of samples, and/or reagents for quantitating the amount of one or more surface markers in a sample, such as, but not limited to, detection reagents, such as antibodies, buffers, substrates for enzymatic staining, chromagens or other materials, such as slides, containers, microtiter plates, and optionally, instructions for performing the methods. Those of skill in the art will recognize many other possible containers and plates and reagents that can be used in accord with the provided methods.

In some embodiments, the kits can be provided as articles of manufacture that include packing materials for the packaging of the cells, antibodies or reagents, or compositions thereof, or one or more other components. For example, the kits can contain containers, bottles, tubes, vial and any packaging material suitable for separating or organizing the components of the kit. The one or more containers may be formed from a variety of materials such as glass or plastic. In some embodiments, the one or more containers hold a composition comprising cells or an antibody or other reagents for use in the methods. The article of manufacture or kit herein may comprise the cells, antibodies or reagents in separate containers or in the same container.

In some embodiments, the one or more containers holding the composition may be a single-use vial or a multi-use vial, which, in some cases, may allow for repeat use of the composition. In some embodiments, the article of manufacture or kit may further comprise a second container comprising a suitable diluent. The article of manufacture or kit may further include other materials desirable from a commercial, therapeutic, and user standpoint, including other buffers, diluents, filters, needles, syringes, therapeutic agents and/or package inserts with instructions for use.

In some embodiments, the kit can, optionally, include instructions. Instructions typically include a tangible expression describing the cell composition, optionally, other components included in the kit, and methods for using such. In some embodiments, the instructions indicate methods for using the cell compositions for administration to a subject for treating a disease or condition, such as in accord with any of the provided embodiments. In some embodiments, the instructions are provided as a label or a package insert, which is on or associated with the container. In some embodiments, the instructions may indicate directions for reconstitution and/or use of the composition.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. For example, in various embodiments about may refer to +/−10%, 5%, 2.5% or 1% of the recited nominal value. Also, reference to “about” a value or parameter herein includes (and describes) embodiments that are directed to that value or parameter per se. For example, description referring to “about X” includes description of “X”.

The term “autologous” as used herein means a cell or tissue that is removed from the same organism to which it is later infused or adoptively transferred.

As used herein, a composition refers to any mixture of two or more products, substances, or compounds, including cells. It may be a solution, a suspension, liquid, powder, a paste, aqueous, non-aqueous or any combination thereof.

As used herein, “optional” or “optional” means that the subsequently described event or circumstance does or does not occur, and that the description includes instances where said event or circumstance occurs and instances where it does not. For example, an optionally substituted group means that the group is unsubstituted or is substituted.

The term “pharmaceutical composition” refers to a composition suitable for pharmaceutical use in a mammalian subject, often a human. A pharmaceutical composition typically comprises an effective amount of an active agent (e.g., tumor reactive T cells, such as those expanded in accord with the provided methods) and a carrier, excipient, or diluent. The carrier, excipient, or diluent is typically a pharmaceutically acceptable carrier, excipient or diluent, respectively.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

As used herein, a “subject” is a mammal, such as a human or other animal, and typically is human. The subject can be male or female and can be any suitable age, including infant, juvenile, adolescent, adult, and geriatric subjects.

The terms “effective amount” or “therapeutically effective amount” refer to a quantity and/or concentration of a therapeutic composition, such as containing cells, e.g. expanded in accord with the provide methods, that when administered to a patient yields any manner in which the symptoms of a condition, disorder or disease or other indication, are ameliorated or otherwise beneficially altered. An effective amount for treating a disease or disorder may be an amount that relieves, lessens, or alleviates at least one symptom or biological response or effect associated with the disease or disorder, prevents progression of the disease or disorder, or improves physical functioning of the patient. In particular aspects, there is a statistically significant inhibition of disease progression as, for example, by ameliorating or eliminating symptoms and/or the cause of the disease. In the case of cell therapy, the effective amount is an effective dose or number of cells administered to a patient. In some embodiments the patient is a human patient.

As used herein, “disease,” disorder”” or “condition” refers to a pathological condition in an organism resulting from cause or condition including, but not limited to, infections, acquired conditions, genetic conditions, and characterized by identifiable symptoms. In particular, it is a condition where treatment is needed and/or desired.

The terms “treating,” “treatment,” or “therapy” of a disease or disorder as used herein mean slowing, stopping or reversing the disease or disorders progression, as evidenced by decreasing, cessation or elimination of either clinical or diagnostic symptoms, by administration of an immunomodulatory protein or engineered cells of the present invention either alone or in combination with another compound as described herein. “Treating,” “treatment,” or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. “Preventing,” “prophylaxis,” or “prevention” of a disease or disorder as used in the context of this invention refers to the administration of an immunomodulatory protein or engineered cells expressing an immunomodulatory protein of the present invention, either alone or in combination with another compound, to prevent the occurrence or onset of a disease or disorder or some or all of the symptoms of a disease or disorder or to lessen the likelihood of the onset of a disease or disorder. For example, in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS).

### VI. EXEMPLARY EMBODIMENTS

Among the provided embodiments are:
1. A pharmaceutical T lymphocyte infiltrating (TIL) composition enriched in tumor reactive T cells, the pharmaceutical composition comprising multiclonal or oligoclonal population of T cells comprising CD4+ and CD8+ T cells from a tumor, wherein the population comprises at least 10 different T cell receptor (TCR) clonotypes each with a frequency in the population of at least 2.0%; and wherein at least 90% of the cells in the composition are CD3+ T cells.
2. The pharmaceutical composition of embodiment 1, wherein at least 11 different TCR clonotypes have a frequency in the population of at least 2.0%.
3. The pharmaceutical composition of embodiment 1, wherein at least 12 different TCR clonotypes have a frequency in the population of at least 2.0%.
4. A pharmaceutical T lymphocyte infiltrating (TIL) composition enriched in tumor reactive T cells, the pharmaceutical composition comprising a multiclonal or oligoclonal population of T cells comprising CD4+ and CD8+ T cells from a tumor, wherein the population comprises at least 10 different T cell receptor (TCR) clonotypes each with a frequency in the population of at least 1.0%; and wherein at least 90% of the cells in the composition are CD3+ T cells.
5. The pharmaceutical composition of embodiment 4, wherein at least 11 different TCR clonotypes have a frequency in the population of at least 1.0%.
6. The pharmaceutical composition of embodiment 4, wherein at least 12 different TCR clonotypes have a frequency in the population of at least 1.0%.
7. The pharmaceutical composition of any of embodiments 1-6, wherein 8 to 15 different T cell receptor (TCR) clonotypes make up at least 50% of the TCR frequency in the population.
8. A pharmaceutical composition enriched in tumor reactive T cells, the pharmaceutical composition comprising a multiclonal population of tumor infiltrating lymphocytes comprising CD4+ and CD8+ T cells from a tumor, wherein 8 to 15 different T cell receptor (TCR) clonotypes make up at least 50% of the TCR frequency in the population.
9. The pharmaceutical composition of any of embodiments 1-8, wherein 9 to 12 different TCR clonotypes make up at least 50% of the TCR frequency in the population.
10. The pharmaceutical composition of any of embodiments 1-9, wherein the TCR clonotypes exhibit reactivity to at least one CD4 antigen and at least one CD8 antigen.
11. The pharmaceutical composition of any of embodiments 1-10, wherein the TCR clonotypes exhibit reactivity for 2 to 100 different peptide antigens.
12. The pharmaceutical composition of any of embodiments 1-11, wherein the TCR clonotypes exhibit reactivity for 10 to 40 different peptide antigens.
13. The pharmaceutical composition of any of embodiments 1-11, wherein the TCR clonotypes exhibit reactivity for 2 to 6 different peptide antigens.
14. The pharmaceutical composition of any of embodiments 1-11, wherein the TCR. clonotypes exhibit reactivity for 2 to 4 peptide antigens.
15. The pharmaceutical composition of any of embodiments 1-11, wherein the TCR clonotypes exhibit reactivity for 2 peptide antigens.
16. The pharmaceutical composition of any of embodiments 1-11 and 13-15, wherein the TCR clonotypes exhibit reactivity for one CD8 antigen and one CD4 antigen.
17. A pharmaceutical composition enriched in tumor reactive T cells, the pharmaceutical composition comprising a multiclonal population of tumor infiltrating lymphocytes comprising CD4+ and CD8+ T cells from a tumor, wherein 10 to 100 different T cell receptor (TCR) clonotypes are present in the population.
18. The pharmaceutical composition of embodiment 17, wherein the TCR clonotypes exhibit reactivity for 10 to 40 different peptide antigens.
19. A pharmaceutical composition enriched in tumor reactive T cells, the pharmaceutical composition comprising a multiclonal population of tumor infiltrating lymphocytes comprising CD4+and CD8+ T cells from a tumor, wherein 20 to 100 different T cell receptor (TCR) clonotypes are present in the population.
20. The pharmaceutical composition of embodiment 19, wherein 20 to 60 different TCR clonotypes are present in the population.
21. A pharmaceutical composition enriched in tumor reactive T cells, the pharmaceutical composition comprising a multiclonal population of tumor infiltrating lymphocytes comprising CD4+ and CD8+ T cells from a tumor, wherein the top 40 TCR clonotypes make up at least 75% of the TCR frequency in the population.
22. The pharmaceutical composition of any of embodiments 5-21, wherein at least 90% of the cells in the population are CD3+ T cells.
23. The pharmaceutical composition of any of embodiments 1-22, wherein the TCR clonotypes exhibit reactivity for at least one CD8 antigen and at least one CD4 antigen.
24. The pharmaceutical composition of any of embodiments 1-23, wherein at least 20% of the CD8+ T cells and/or at least 20% of the CD4+ T cells in the composition exhibit neoantigen reactivity.
25. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein at least 20% of the CD8+ T cells and/or at least 20% of the CD4+ T cells in the composition exhibit neoantigen reactivity.
26. The pharmaceutical composition of embodiment 24 or embodiment 25, wherein at least 25% of the CD8+ T cells and/or at least 20% of the CD4+ T cells in the composition exhibit neoantigen reactivity.
27. The pharmaceutical composition of embodiment 24 or embodiment 25, wherein at least 30% of the CD8+ T cells and/or at least 20% of the CD4+ T cells in the composition exhibit neoantigen reactivity.
28. The pharmaceutical composition of embodiment 24 or embodiment 25, wherein at least 40% of the CD8+ T cells and/or at least 30% of the CD4+ T cells in the composition exhibit neoantigen reactivity.
29. The pharmaceutical composition of any of embodiments 19-28, wherein neoantigen reactivity is determined in a co-culture assay with peptide loaded autologous APCs (e.g., as described in Example 2) by one or more of: upregulation of CD134 and CD137, IFN-γ production, TNF-alpha production, granzyme B production or degranulation, optionally wherein degranulation is determined based on CD107 expression.
30. The pharmaceutical composition of any of embodiments 1-29, wherein the TIL composition is characterized by at least a 1.5-fold increased percentage of cells positive for CD134 and CD137 compared to a bulk TIL population in a co-culture assay with peptide loaded autologous APCs, optionally at least a 2-fold, at least a 3-fold or at least a 4-fold increase in CD134 and CD137 positive cells compared to a bulk TIL population.
31. The pharmaceutical composition of any of embodiments 1-11, wherein greater than 30% of the cells in the TIL composition are positive for CD134 and CD137 in a co-culture assay with peptide loaded autologous APCs, optionally greater than about 35%, greater than about 40%, or greater than about 45% of cells are positive for CD134 and CD137.
32. The pharmaceutical composition of any of embodiments 1-31, wherein greater than 48% of the cells in the TIL composition are positive for CD134 and CD137 in a co-culture assay with peptide loaded autologous APCs, optionally greater than about 50%, greater than about 60%, or greater than about 70% of cells are positive for CD134 and CD137.
33. The pharmaceutical composition of any of embodiments 1-32, wherein, the TIL composition is characterized by at least one of the following criteria in an in vitro co-culture assay with peptide loaded autologous APC:
   i) IFN-γ production that is greater than 1,000 pg/mL;
   (ii) TNF-alpha production that is greater than 100 pg/mL;
   (iii) greater than 10% CD107a+ cells; and
   iv) granzyme B production that is greater than 10,000 pg/mL.
34. The pharmaceutical composition of any of embodiments 1-33, wherein, the TIL composition is characterized by at least one of the following criteria in an in vitro co-culture assay with peptide loaded autologous APC:
   i) IFN-γ production that is greater than 100,000 pg/mL;
   (ii) TNF-alpha production that is greater than 250 pg/mL;
   (iii) greater than 10% CD107a+ cells; and
   iv) granzyme B production that is greater than 50,000 pg/mL.
35. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following criteria in an in vitro co-culture assay with peptide loaded autologous APC:
   i) IFN-γ production that is greater than 1,000 pg/mL;
   (ii) TNF-alpha production that is greater than 100 pg/mL;
   (iii) greater than 10% CD107a+ cells; and
   (iv) granzyme B production that is greater than 10,000 pg/mL.
36. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following criteria in an in vitro co-culture assay with peptide loaded autologous APC:
   i) IFN-γ production that is greater than 100,000 pg/mL;
   (ii) TNF-alpha production that is greater than 250 pg/ml;
   (iii) greater than 10% CD107a+ cells; and
   (iv) granzyme B production that is greater than 50,000 pg/mL.
   37. The pharmaceutical composition of embodiments 33-36, wherein the TIL composition is characterized by at least two of criteria (i)-(iv).
38. The pharmaceutical composition of embodiments 33-36, wherein the TIL composition is characterized by at least three of criteria (i)-(iv).
39. The pharmaceutical composition of embodiments 33-36, wherein the TIL composition is characterized by criteria (i)-(iv).
40. The pharmaceutical composition of any of embodiments 29-39, wherein the TIL composition is characterized by IFN-γ production that is greater than 2,500 pg/mL, greater than 5,000 pg/mL, greater than 10,000 pg/mL, greater than 25,000 pg/mL, greater than 50,000 pg/mL, greater than 100,000 pg/mL, greater than 200,000 pg/mL, greater than 250,000 pg/mL, greater than 500,000 pg/mL, or greater than 1,000,000 pg/mL.
41. The pharmaceutical composition of any of embodiments 29-40, wherein the TIL composition is characterized by IFN-γ production that is greater than 250,000 pg/mL, greater than 500,000 pg/mL, or greater than 1,000,000 pg/mL.
42. The pharmaceutical composition of any of embodiments 29-41, wherein the TIL composition is characterized by TNF-alpha production that is greater than 200 pg/mL, greater than 500 pg/mL, greater than 1000 pg/mL, or greater than 2000 pg/mL.
43. The pharmaceutical composition of any of embodiments 29-42, wherein the TIL composition is characterized by TNF-alpha production that is greater 500 pg/mL, greater than 1000 pg/mL, or greater than 2000 pg/mL.
44. The pharmaceutical composition of any of embodiments 29-43, wherein the TIL composition is characterized by greater than 15% CD107a+ cells, greater than 20% CD107a cells, or greater than 25% CD107a+ cells.
45. The pharmaceutical composition of any of embodiments 29-44, wherein the TIL composition is characterized by granzyme B production that is greater than 15,000 pg/mL, greater than 25,000 pg/mL, greater than 50,000 pg/mL, greater than 100,000 pg/mL, greater than 200,000 pg/mL, greater than 300,000 pg/mL, greater than 400,000 pg/mL or greater than 500,000 pg/mL.
46. The pharmaceutical composition of any of embodiments 29-45, wherein the TIL composition is characterized by granzyme B production that is greater than 200,000 pg/mL, greater than 300,000 pg/mL, greater than 400,000 pg/mL or greater than 500,000 pg/mL.
47. The pharmaceutical composition of any of embodiments 1-46, wherein, the TIL composition is characterized by at least one of the following criteria in an in vitro co-culture assay with peptide loaded autologous APC:
   i) IFN-γ that is 50-fold or higher than a bulk TIL composition that is not enriched for tumor reactive T cells;
   ii) TNF-α that is 300-fold or higher than a bulk TIL composition that is not enriched for tumor reactive T cells; or
   iii) granzyme B that is 15-fold or higher than a bulk TIL composition that is not enriched for tumor reactive T cells.
48. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following criteria in an in vitro co-culture assay with peptide loaded autologous APC:
   i) IFN-γ that is 50-fold or higher than a bulk TIL composition that is not enriched for tumor reactive T cells;
   ii) TNF-α that is 300-fold or higher than a bulk TIL composition that is not enriched for tumor reactive T cells; or
   iii) granzyme B that is 15-fold or higher from a bulk TIL composition that is not enriched for tumor reactive T cells.
49. The pharmaceutical composition of embodiment 47 or embodiment 48, wherein the TIL composition is characterized by criteria (i) and (ii).
50. The pharmaceutical composition of embodiment 47 or embodiment 48, wherein the TIL composition is characterized by criteria (i) and (iii).
51. The pharmaceutical composition of embodiment 47 or embodiment 48, wherein the TIL composition is characterized by criteria (ii) and (iii).
52. The pharmaceutical composition of embodiment 47 or embodiment 48, wherein the TIL composition is characterized by criteria (i), (ii) and (iii).
53. The pharmaceutical composition of any of embodiments 1-52, wherein the composition is characterized by a greater number of CD4+ T cells than CD8+ T cells.
54. The pharmaceutical composition of any of embodiments 1-52, wherein a ratio of CD4+ T cells to CD8+ T cells in the composition is between 5:1 to 1:5.
55. The pharmaceutical composition of any of embodiments 1-52, wherein a ratio of CD4+ T cells to CD8+ T cells in the composition is between 5:1 to 50:1, between 5:1 to 25:1, between 5:1 to 20:1, between 5:1 to 15:1, between 5:1 to 10:1, between 10:1 to 50:1, between 10:1 to 25:1, between 10:1 to 20:1, between 10:1 to 15:1, between 15:1 to 50:1, between 15:1 to 25:1, between 15:1 to 20:1, between 20:1 to 50:1, between 20:1 to 25:1 or between 25:1 to 50:1.
56. The pharmaceutical composition of any of embodiments 1-55, wherein a ratio of CD4+ T cells to CD8+ T cells in the composition is at or about 10:1 to 25:1, optionally at or about 20:1.
57. The pharmaceutical composition of any of embodiments 1-56, wherein greater than 50% of the CD3+ T cells, optionally greater than 50% of the CD4 and CD8+ T cells, express markers of an effector memory phenotype.
58. The pharmaceutical composition of any of embodiments 1-57, wherein greater than 75% of the CD3+ T cells, optionally greater than 75% of the CD4 and CD8+ T cells, express markers of an effector memory phenotype.
59. The pharmaceutical composition of any of embodiments 1-58, wherein greater than 80% of the CD3+ T cells, optionally greater than 80% of the CD4 and CD8+ T cells, express markers of an effector memory phenotype.
60. The pharmaceutical composition of any of embodiments 1-58, wherein greater than 85% of the CD3+ T cells, optionally greater than 85% of the CD4 and CD8+ T cells, express markers of an effector memory phenotype.
61. The pharmaceutical composition of any of embodiments 1-58, wherein greater than 90% of the CD3+ T cells, optionally greater than 90% of the CD4 and CD8+ T cells, express markers of an effector memory phenotype.
62. The pharmaceutical composition of any of embodiments 58-61, wherein the effector memory phenotype is characterized by surface marker expression of one or more of CD45RA. CD45RO+, CD62L⁻, CCR7−, CD28− and CD27−.
63. The pharmaceutical composition of any of embodiments 58-62, wherein the effector memory phenotype is characterized by surface marker expression CD45RA, CD45RO+, CD62L−, and CCR7−.
64. The pharmaceutical composition of any of embodiments 58-62, wherein the effector memory phenotype is characterized by surface marker expression CD45RA⁻, CD45RO+, CD62L⁻, CCR7−, CD28− and CD27−.
65. The pharmaceutical composition of any of embodiments 58-62, wherein the effector memory phenotype is characterized by surface marker expression CD45RA⁻ and CCR7⁻.
66. The pharmaceutical composition of any of embodiments 1-65, wherein greater than 95% of the CD4+ and CD8+ T cells in the composition are PD-1.
67. The pharmaceutical composition of any of embodiments 1-66, wherein greater than 80% of the CD4+ and CD8+ T cells in the composition LAG3⁻.
68. The pharmaceutical composition of any of embodiments 1-67, wherein the number of cells in the composition, or of viable cells thereof, is at least 2×10⁷ cells.
69. The pharmaceutical composition of any of embodiments 1-68, wherein the number of cells in the composition, or of viable cells thereof, is between at or about 2×10⁷ cells and 20×10⁹ cells, 2×10⁷ cells and 10×10⁹ cells, 2×10⁷ cells and 2×10⁹ cells, 2×10⁷ cells and 2×10⁸ cells, 2×10⁸ cells and 20×10⁹ cells, 2×10⁸ cells and 10×10⁹ cells, 2×10⁸ cells and 2×10⁹ cells, 2×10⁹ cells and 20×10⁹ cells, 2×10⁹ cells and 10×10⁹ cells, or 10×10⁹ cells and 20×10⁹ cells, each inclusive.
70. The pharmaceutical composition of any of embodiments 1-69, wherein the pharmaceutical composition is for treatment of a patient's tumor.
71. The pharmaceutical composition of any of embodiments 1-70, wherein the tumor is a colorectal cancer (CRC) tumor, a melanoma tumor, a non-small cell lung cancer (NSCLC) tumor, or an ovarian cancer tumor.
72. The pharmaceutical composition of any of embodiments 1-71, wherein the tumor is from a human subject.
73. The pharmaceutical composition of embodiment 72, wherein the pharmaceutical composition is for antologous adoptive therapy to the human subject.
74. The pharmaceutical composition of any of embodiments 1-73, comprising a pharmaceutically acceptable excipient.
75. The pharmaceutical composition of any of embodiments 1-74, comprising a cryoprotectant.
76. The pharmaceutical composition of any of embodiments 1-75, wherein the composition is a liquid composition.
77. The pharmaceutical composition of embodiment 76, wherein the composition had been frozen and thawed.
78. The pharmaceutical composition of any of embodiments 1-77, wherein the volume of the composition is between 1 mL and 500 mL.
79. The pharmaceutical composition of any of embodiments 1-78, wherein the composition is frozen.
80. A pharmaceutical T lymphocyte infiltrating (TIL) composition enriched in tumor reactive T cells for treatment of a patient's tumor, the pharmaceutical composition comprising a multiclonal or oligoclonal population of T cells comprising CD4+ and CD8+ T cells from the patient's tumor, wherein the population comprises at least 10 different T cell receptor (TCR) clonotypes each with a frequency in the population of at least 2.0%; and wherein at least 90% of the cells in the composition are CD3+ T cells.
81. A pharmaceutical T lymphocyte infiltrating (TIL) composition enriched in tumor reactive T cells for treatment of a patient's tumor, the pharmaceutical composition comprising a multiclonal or oligoclonal population of T cells comprising CD4+ and CD8+ T cells from the patient's tumor, wherein the population comprises at least 10 different T cell receptor (TCR) clonotypes each with a frequency in the population of at least 1.0%; and wherein at least 90% of the cells in the composition are CD3+ T cells.
82. The pharmaceutical composition of any of embodiments 1-81, wherein the TIL composition is produced by an ex vivo method comprising expansion of tumor-reactive T cells from a donor subject that have been co-cultured with autologous antigen presenting cells and peptide neoantigens.
83. The pharmaceutical composition of any of embodiments 1-82, wherein the TIL composition is produced by a method comprising:
   a. providing dissociated tumor cells from a tumor obtained from a donor subject, wherein the dissociated tumor cells are a first population of T cells that comprise CD4+ and CD8+ T cells;
   b. culturing the first population of T cells with recombinant IL-2 added at a concentration between 3000 IU/mL and 6000 IU/mL, inclusive, for 14 to 28 days to produce a second population of T cells;
   c. co-culturing the second population of T cells for 12 to 48 hours with autologous antigen presenting cells (APCs) with recombinant IL-2 added at a concentration of 100 IU/mL to 1000 IU/mL to produce a third population of T cells, wherein the APCs are loaded with a pool of peptide neoantigens from the tumor, wherein each peptide is 13-40 amino acids in length and is loaded at a concentration of 100 ng/ml per peptide, and wherein the ratio of the second population of T cells to APCs is 2:1 to 10:1;
   d. selecting cells from the third population of T cells that are surface positive for CD134 and/or CD137 to produce a fourth population of T cells; and e. expanding tumor infiltrating lymphocytes (TILs) by incubating the fourth population of T cells with irradiated human peripheral blood mononuclear cells (iPBMCs) at a ratio of 100 to 500 IPBMC to cells of the fourth population of T cells with recombinant IL-2 added at a concentration between 3000 IU/mL and 6000 IU/mL, inclusive, and 10 to 50 ng/ml anti-CD3 antibody (OKT3) for 12 to 16 days to produce a therapeutic composition of TILs enriched in tumor reactive cells.
84. A frozen composition comprising the pharmaceutical composition of any of embodiments 1-83 and a cryoprotectant.
85. A method of producing a T lymphocyte infiltrating (TIL) composition enriched in tumor reactive T cells, the method comprising:
   a. providing dissociated tumor cells from a tumor obtained from a donor subject, wherein the dissociated tumor cells are a first population of T cells that comprise CD4+ and CD8+ T cells;
   b. culturing the first population of T cells with recombinant IL-2 added at a concentration between about 3000 IU/mL and 6000 IU/mL, inclusive, for about 14 to 28 days to produce a second population of T cells;
   c. co-culturing the second population of T cells for about 12 to 48 hours with autologous antigen presenting cells (APCs) with recombinant IL-2 added at a concentration of 100 IU/mL to 1000 IU/mL to produce a third population of T cells, wherein the APCs are loaded with a pool of peptide neoantigens from the tumor, wherein each peptide is 13-40 amino acids in length and is loaded at a concentration of 100 ng/ml per peptide, and wherein the ratio of the second population of T cells to APCs is about 2:1 to 10:1;
   d. selecting cells from the third population of T cells that are surface positive for CD134 and/or CD137 to produce a fourth population of T cells; and
   e. expanding tumor infiltrating lymphocytes (TILs) by incubating the fourth population of T cells with irradiated human peripheral blood mononuclear cells (iPBMCs) at a ratio of about 100 to 500 iPBMC to cells of the fourth population of T cells with recombinant IL-2 added at a concentration between about 3000 IU/mL and 6000 IU/mL, inclusive, and 10 to 50 ng/ml anti-CD3 antibody (OKT3) for 12 to 16 days to produce a therapeutic composition of TILs enriched in tumor reactive cells.
86. A method of treating a subject having a cancer, the method comprising administering to a subject having a tumor a therapeutic dose of the composition of any of embodiments 1-84.
87. The method of embodiment 86, wherein the therapeutically effective dose is between about 1×10⁹ and 10×10⁹ T cells.
88. The method of embodiment 86, wherein the therapeutically effective dose is from more than 1 million to less than 100 million T cells per kilogram of body weight.
89. The method of embodiment 86, wherein the therapeutically effective dose is from more than 1 million to less than 10 million T cells per kilogram of body weight.
90. The method of embodiment 86, wherein the therapeutically effective dose is from at or about 10 million to at or about 50 million T cells per kilogram of body weight.
91. The method of any of embodiments 86-90, wherein the cells of the therapeutic composition are antologous to the subject.

### VII. EXAMPLES

The following examples are included for illustrative purposes only and are not intended to limit the scope of the invention.

### Example 1 Methods for Producing Tumor Infiltrating Lymphocytes for Multiple Indications

A tumor infiltrating lymphocyte (TIL) product with enriched reactivity to mutations expressed in neoantigens was generated from patient material. Without wishing to be bound by theory, the enrichment process as described herein enriches for tumor reactive TIL while also reducing the frequency of bystander TIL in the final product. In this process depicted in FIG. 1, tumor infiltrating lymphocytes (TIL) were expanded from a cryopreserved dissociated colorectal cancer (CRC), non-small cell lung cancer (NSCLC), melanoma, or ovarian cancer tumor sample and antigen presenting cells (APCs) were generated from patient-matched blood. Mutations were predicted and peptides containing the potential neoantigens were manufactured. TIL from cryopreserved dissociated tumor cells of a patient were enriched for neoantigen reactivity by fluorescence single cell sorting (FACS) following coculture with autologous APCs pulsed with predicted peptides. The sorted neoantigen positive TIL (tumor reactive enriched-TIL composition) and TIL from the negative fraction (bystander cells) were expanded using a Rapid Expansion Protocol (REP). As a control, bulk TIL from unselected (e.g., no sort) were also expanded using REP.

### A. Selection and Tumor-Reactive Enrichment for Selected TIL

### 1. Neoantigen Identification, Synthesis

APCs were isolated and expanded from the patient's autologous blood. B cells were isolated from autologous PBMCs using an EasyStep Human CD19 Positive Selection Kit (e.g., StemCell Cat. No. 17854). The isolated B cells were cultured with 240 ng/ml multimeric CD40L and 50 IU/mL IL-4 and incubated at 37° C., 5% CO2 for about 14 days followed by their cryopreservation. A 50% media change was performed on Day 5 and cytokines were replenished every 3 days afterwards. Alternatively, B cells are immortalized in the event of limited blood volume. Immortalization of B cells can be attained by infecting the culture with Epstein Bar Virus or engineering the B cells with BCL-6 and BCL-XL genes. If an apheresis product is available, dendritic cells (DCs) differentiated from monocytes can be used as APCs.

Whole exome and transcriptome (RNA sequencing) was performed on tumor tissue and autologous PBMCs and used to predict neoantigen mutations. Mutation-derived neoantigens were identified by sequencing and up to 192 13-40 amino acid peptides (e.g., 25nmer) containing the mutation were generated (synthesized by GenScript).

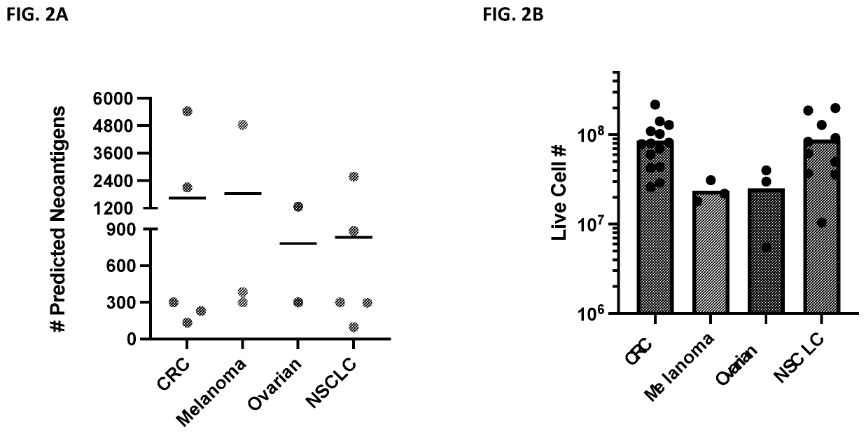

While CRC samples accounted for half of the tumors, the tumor mutational burden within this population varied significantly, ranging from 132 to 5436 mutations. As shown in FIG. 2A, the tumor mutational burden was variable both within and across tumor indications.

### 2. Processing of Samples and Pre-REP

Frozen tumor fragments or dissociated tumor cells (e.g., as little as 1×10⁷ dissociated tumor cells) from a primary tumor sample were thawed and suspended in complete TIL media, e.g., made with Gibco™ CTS™ OpTmizer™ T Cell Expansion SFM (Life Technologies Cat. No. A37050001) to which was added 25 mL CTS™ OpTmizer expansion supplement, 50 mL CTS™ Immune Cell SR (Life Technologies Cat. No. A2596101), 0.5 mL gentamicin (50 mg/ml; Life Technologies Cat. No. 15-750-060), 10 mL of 100× GlutaMAX Supplement (Life Technologies Cat. No. 35-050-061), and 20 mL of Human AB Serum (Sigma Aldrich Cat. No. H4522-100mL). The suspended cells in complete media were added at Day 0 to a G-Rex6M well cell culture plate with 3000 IU/mL or 6000 IU/mL human recombinant IL-2 and cells were incubated for pre-rapid expansion (pre-REP) by culture at 37° C., 5% CO 2 for 27 days. A 50% media exchange was performed every 5 days with the complete media containing human recombinant IL-2 to the same final concentration. In some cases, fresh tumors were cut into fragments (1-3mm³) or dissociated and cultured into 24well G-Rex plates in TIL media containing 5% Human AB serum and 6000 IU/mL IL-2 in a primary expansion (preREP) for 14-28 days. The pre-REP TIL were cryopreserved in Cryostor CD10 with 5% Human Serum Albumin and frozen at −80° C. Serum as described above is included to improve the success rate of the pre-REP, however, the process can be performed in serum-free media.

Successful TIL expansion was achieved in 31 out of 34(91%) tumors using both tumor fragments and dissociated cells as described above (i.e., 14/17 CRC cultures, 10/10 NSCLC cultures, 3/3 ovarian cultures, and 3/3 melanoma cultures). It was observed that pre-REP expansion was greatest in CRC and NSCLC tumors, with mean yields of 8.67e7 and 8.86e7 respectively (FIG. 2B). The three CRC samples that failed to expand were all initiated from dissociated tumor cell suspensions in TIL media lacking human serum.

### 3. Co-culture

B cells were thawed and following a 24-hour rest were loaded with a peptide pool of 190 peptides containing predicted mutations at a concentration of 100 ng/ml per peptide. In order to stimulate any tumor reactive CD4 and/or CD8+ TIL, the TIL were co-cultured with APCs that have been pulsed with the identified peptides as described above. Briefly, the following day, about 3×10⁷ pre-REP expanded TIL were used for co-culture with the peptide-pulsed antigen presenting cells (APCs). The co-cultures were seeded at a range of ratios from 1:1to 5:1 TIL: B cells and cultured for about 20 hours (e.g., overnight) in the presence of 300 IU/mL IL-2. Cells were stained with CD134 and CD137 and cells were selected that expressed CD134 and/or CD137 above the fluorescence minus one (FMO) control after about 20 hours of co-culture with peptide-pulsed APC using a FACSAria cell sorter. For comparison, also collected were unselected bystander cells from the negative fraction (lacking expression of both markers CD134 and CD137) and bulk TIL that were not subjected to the sort selection.

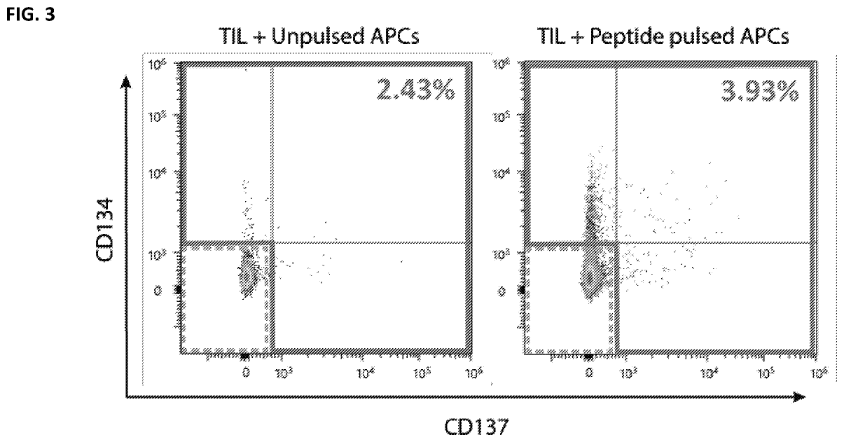

Flow cytometry analysis of cells for surface markers CD137 and CD134 showed that about 3.93% of CD8+ and CD4+ T cells after the co-culture expressed either CD137 or CD134 above the FMO (FIG. 3). Peptide loaded co-cultures show increased expression of CD134 and CD137 compared to unloaded co-cultures, 2.43% vs 3.9% respectively.

### B. Rapid Expansion Protocol

For rapid expansion protocol (REP), the selected TIL from above, unselected bystander cells from above and bulk TIL were separately incubated with irradiated human peripheral blood mononuclear cells (iPBMCs) in a G-Rex6M or G-Rex 24 well plate at a ratio of about 100:1 to 200:1 iPBMC to TIL, with 3000-6000 IU/mL human recombinant IL-2 and 30 ng/ml anti-CD3 antibody (OKT3). The cells were expanded for approximately 14 days. The cells were incubated at 37° C., 5% CO 2 until a threshold number of cells of about 4×10⁸ cells were generated (e.g., about 13 or 14 days). On Days 5 and 10, a 50% media exchange was performed with the complete media containing human recombinant IL-2 to a final concentration of 6000 IU/mL, and on Day 10 cells were split into multiple wells. The expanded TIL were cryopreserved in 95% Cryostor CD10 with 5% Human Serum Albumin and frozen at −80° C. The TIL also may be cryopreserved in 50% Cryostor CD10 with 5% Human Serum Albumin, 45% Plasmalyte A, and frozen at −80° C.

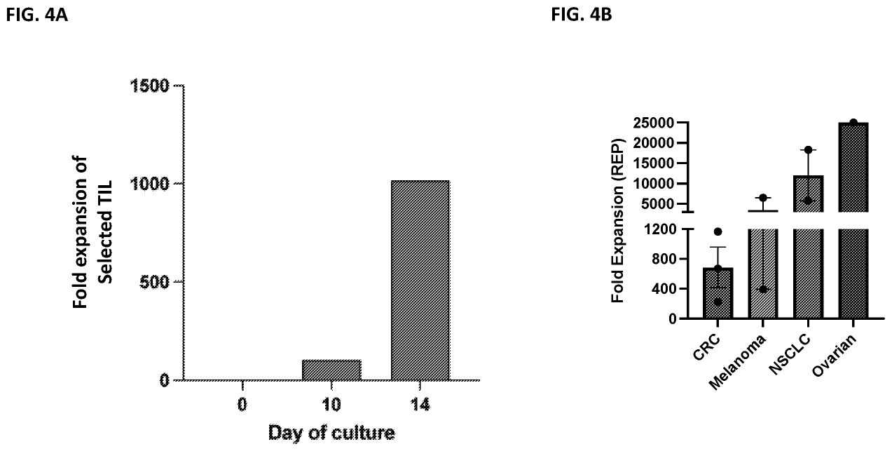

The fold expansion of sorted TIL products after 10 and 14 day REP was calculated by dividing the total number of live cells at each time point by the number of live cells seeded on Day 1. As shown in FIG. 4A, for a particular CRC donor, the selected tumor reactive-enriched TIL population expanded over 1000-fold during REP. As shown in FIG. 4B, TIL products were successfully expanded during REP from donors with different cancer indications tested, with NSCLC and ovarian TIL showing the greatest expansion.

The above process also can be adapted to use fresh tissue fragments instead of a frozen dissociated tumor, serum-free media, autologous dendritic cells instead of T cells, and can be adapted for larger scale manufacturing such as using a GRex 500M for REP.

### Example 2: Characterization of Tumor Reactive Enriched-TIL Composition

TIL products were produced as described in Example 1, and were assessed for phenotype, TCR repertoire and functional characterization.

### A. Til Phenotype

Flow cytometry analysis for surface markers CD3, CD56, CD4 and CD8 was used to assess phenotype of TILs in the selected REP composition. TIL were also characterized to quantify composition, memory subsets, as well as activation and/or exhaustion. T cell memory populations were defined based on the expression of CD45RA and CCR7 within the CD4 and CD8 subsets, where TEM=effector memory T cells (CD45RA-CCR7−), TCM =central memory T cells (CD45RA-CCR7+), TSCM =Naïve/stem cell memory T cells (CD45RA+CCR7+) and TEMRA =effector T(CD45RA+CCR7−) of selected TIL as defined by CCR7 and CD45RA.

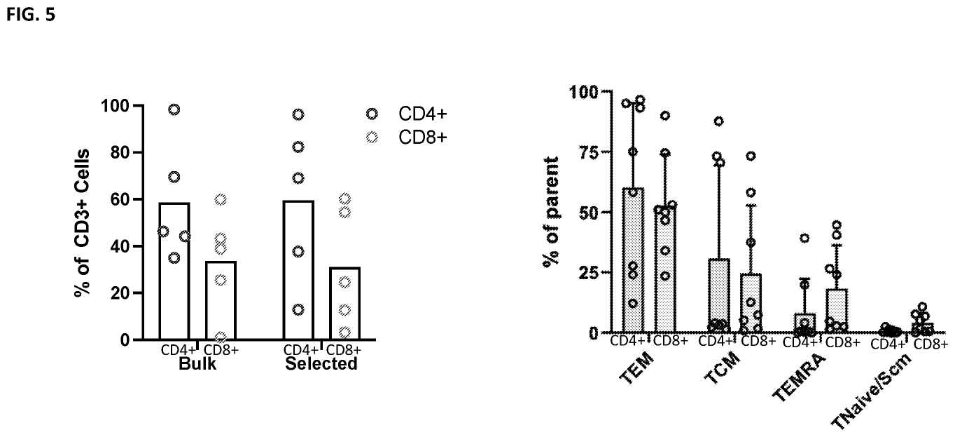

It was observed in FIG. 5 that neoantigen selected TIL were phenotypically similar to bulk TIL. It was observed for both bulk and selected TIL that the majority of cells were TEM, and a smaller fraction of cells were TCM. Minimal TEMRA and TNaive/SCM cells were quantified following sorting. In one exemplary CRC donor, selected TIL composition after REP was about 92% CD3+ of which 91.8% were CD4+ T cells and 4.35% were CD8+ T cells. Also, by flow cytometry analysis of cell surface markers CD45RO, CD45RA, CD62L, CCR7, CD27 and CD28, >90% of CD4 and CD8 cells expressed markers of an effector memory phenotype.

### B. TCR Repertoire

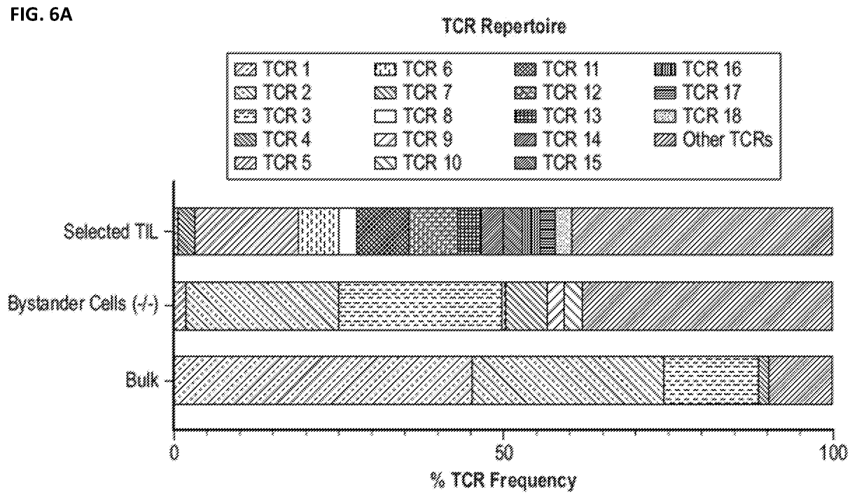

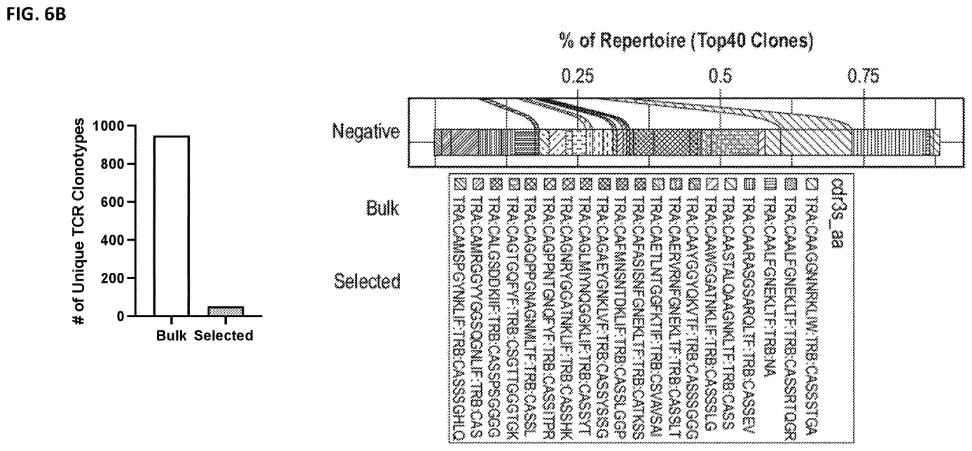

Single cell TCR sequencing (scRNAseq) was performed on 2000 cells from each of the expanded bulk TIL composition, selected TIL composition and bystander (unselected negative fraction) TIL composition using the 10× genomics platform. Enrichment of several clonotypes in the selected TIL composition was observed, including some that were very low frequency in the bulk population (FIG. 6A). By comparison, the profile of the negative fraction bystander cells was similar to the bulk TIL. Selected TIL displayed a reduction in TCR diversity compared to bulk TIL, with 52 clonotypes identified in selected TIL compared to 948 in bulk TIL, indicative of successful selection (FIG. 6B). Analysis of the Top 40 most frequent TCR clonotypes shows enrichment of multiple unique TCRs within the selected TIL population relative to bulk and negatively selected TIL, with these unique TCRs making up the majority of clonotypes within the selected TIL product. The results reveal enrichment of low frequency clones in the selected TIL composition after REP that were undetectable in the bulk REP. The results also establish that the TILs have multiple clonotypes for a given antigen. These data additionally demonstrate that the method herein can produce selected TIL enriched for neoantigen reactivity for multiple tumor types including CRC, melanoma, ovarian, and NSCLC.

### C. Functional Characterization

The selected TIL composition, bystander TIL (unselected negative fraction) and bulk TIL after REP were assessed for T cell activation reactivity to neoantigens following culture alone or in a co-culture assay by co-culture of cells in the TIL composition at an effector: target ratio of 5:1 with autologous peptide loaded or unloaded B cells. Unloaded co-cultures were used as negative controls. T cell activation was quantified by CD134 and CD137 expression, in addition to intracellular IFN-γ production after 5 hours of co-culture in the presence of Golgi Plug and GolgiStop. The reactivity of selected TIL was compared to the bulk TIL that were unselected (no sort), as well as to the negative fraction bystander cells from the sort selection.

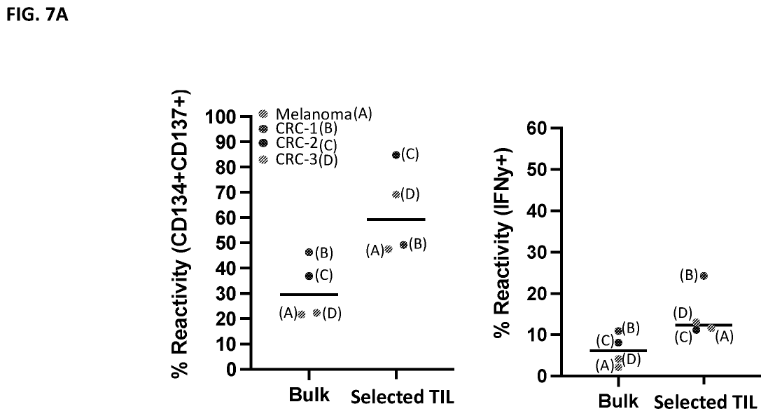

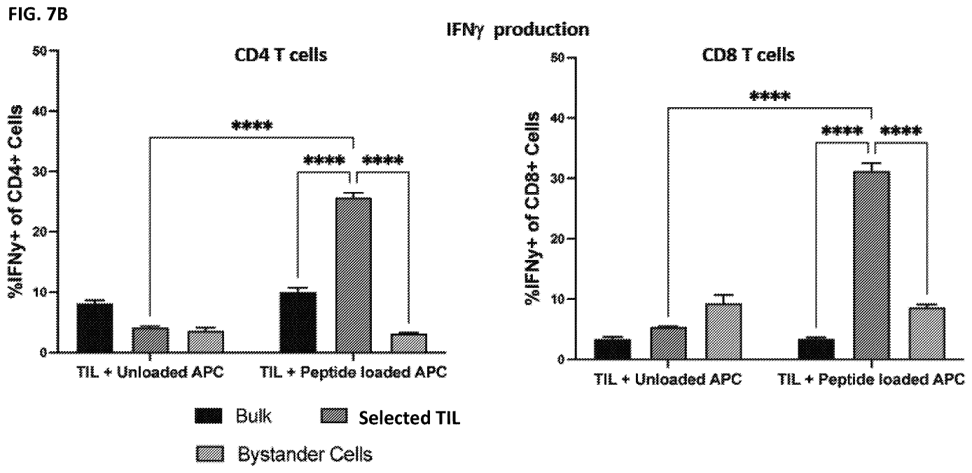

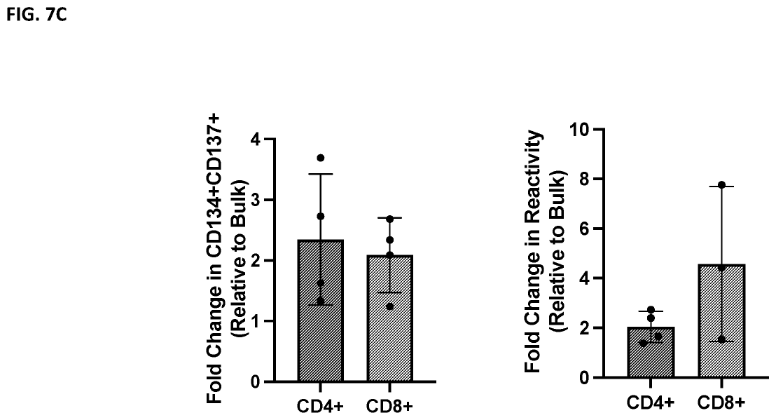

Selected TIL compositions were observed to display significant upregulation of CD134 and CD137 and increased IFN-γ production (FIG. 7A) upon co-culture with peptide loaded APCs relative to bulk TIL. As shown in FIG. 7B, the selected TIL composition displayed enhanced reactivity to neoantigen peptides relative to bulk TILs and negative fraction as determined by percent of CD 4+or CD8+ T cells positive for IFNy. In response to co-culture, 31.2% of CD8 cells and 25.6% of CD4 cells in the selected TIL product were positive for IFN-γ by intracellular cytokine staining. Increased neoantigen reactivity, measured as the fold change in T cell activation (CD134+CD137+) and IFNγ production of selected TIL relative to bulk TIL, was observed in both the CD4 and CD8 compartments (FIG. 7C). The selected TIL population co-cultured with peptide loaded APC showed significant increases in IFN-γ production relative to unloaded and peptide loaded bulk and negative fraction TIL cultures underscoring their specific reactivity.

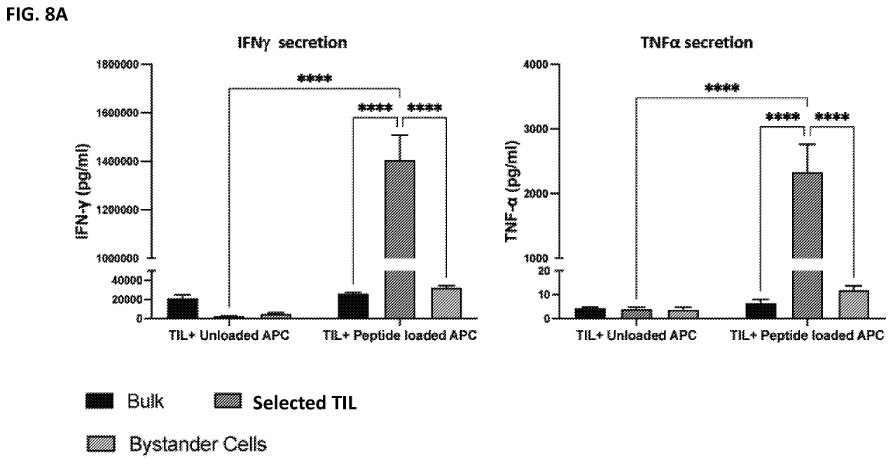

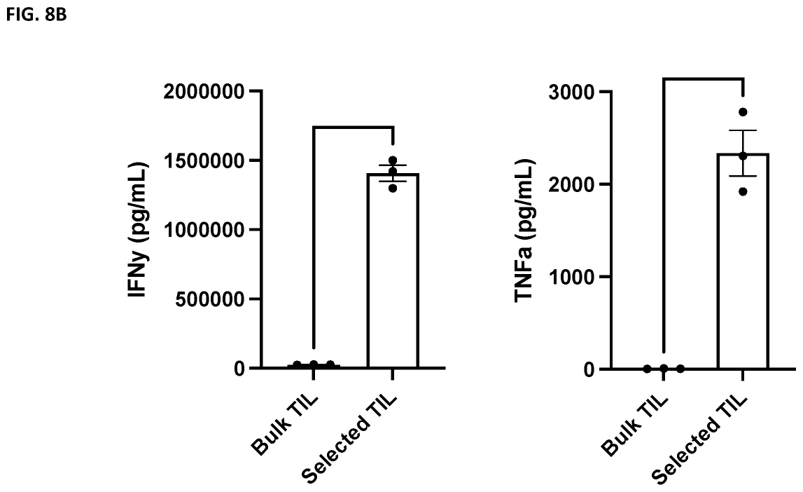

The reactivity to neoantigens of the cultures above also was determined by monitoring cytokine secretion (IFN-γ, TNF-α) of REP expanded selected TILs, bystander cells (unselected negative fraction) and bulk TILs following coculture for 24 hours at an effector: target ratio of 5:1 with autologous peptide loaded or unloaded B cells. Supernatants were collected and analyzed using LEGENDPlex cytometric bead array. The selected TIL composition produced significantly more IFN-γ and TNF-α than bulk TIL upon co-culture with loaded B cells demonstrating enhanced reactivity specific to the peptide pool (FIG. 8A). IFN-γ and TNF-α were 53 and 360-fold higher in the co-culture supernatants of selected TIL compared to bulk TIL, respectively (FIG. 8B). Selection of patient tumor-reactive TCRs results in superior cytokine expression in the selected TIL composition in comparison to Bulk TIL in response to patient-tumor specific neoantigens.

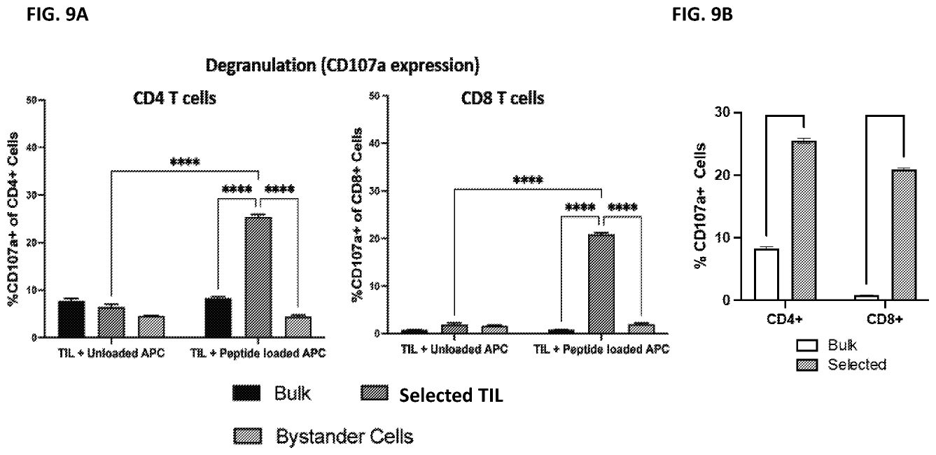

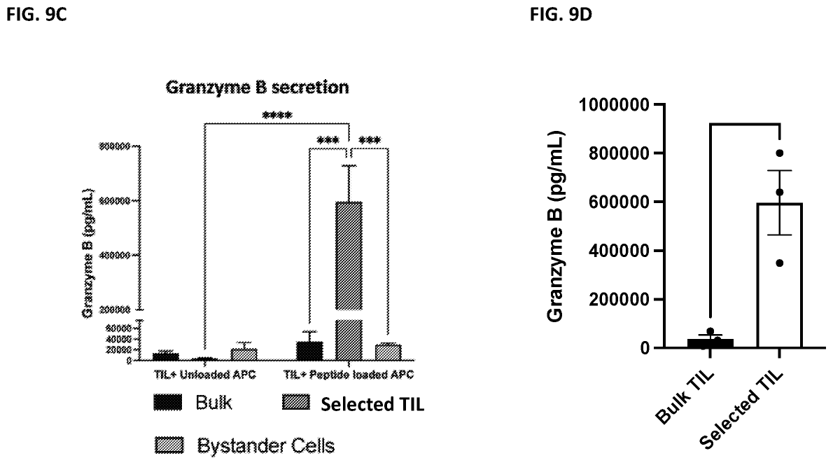

As a surrogate of T cell killing, the reactivity to neoantigens also was determined by T cell degranulation assay as determined by CD107a expression. Selected TILs, bystander cells (unselected negative fraction) and bulk TILs after REP were co-cultured at an effector: target ratio of 5:1 with autologous peptide loaded or unloaded B cells. CD107a expression was assessed after 5 hours of co-culture by flow cytometry. TIL only and unloaded co-cultures were used to establish baseline CD107a expression. As a measure of killing potential, 20.9% of CD8 cells in the selected TIL composition degranulated in co-culture based on CD107 expression. The selected TILs produced significantly more CD107a than bulk TILs or bystander cells, demonstrating enhanced reactivity that is specific to the peptide pool (FIG. 9A and FIG. 9B).

Granzyme B production was quantified from REP expanded selected TILs, bystander cells (unselected negative fraction) and bulk TILs following coculture for 24 hours at an effector: target ratio of 5:1 with autologous peptide loaded or unloaded B cells. Supernatants were collected and analyzed using LEGENDPlex cytometric bead array. Expression of the cytolytic enzyme Granzyme B represents a key mechanism by which T cells kill tumor cells. The selected TIL composition produced significantly more Granzyme B than bulk TIL upon co-culture with loaded B cells demonstrating enhanced reactivity specific to the peptide pool (FIG. 9C and FIG. 9D). Granzyme B secretion was increased 16.5-fold over bulk TIL.

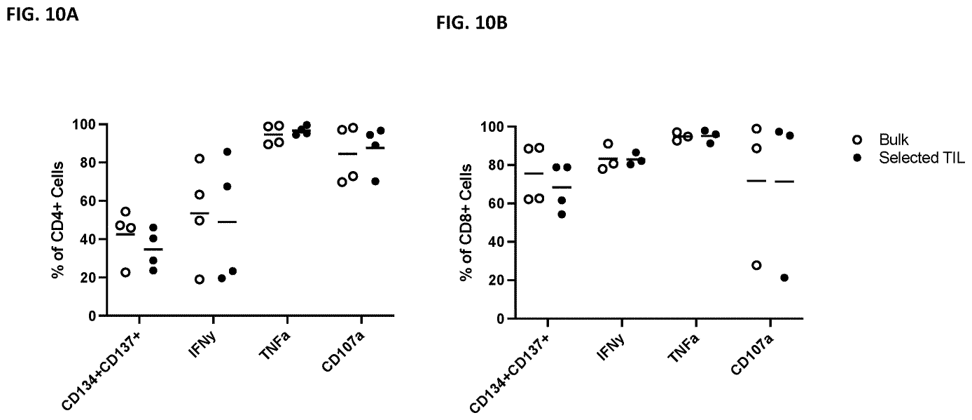

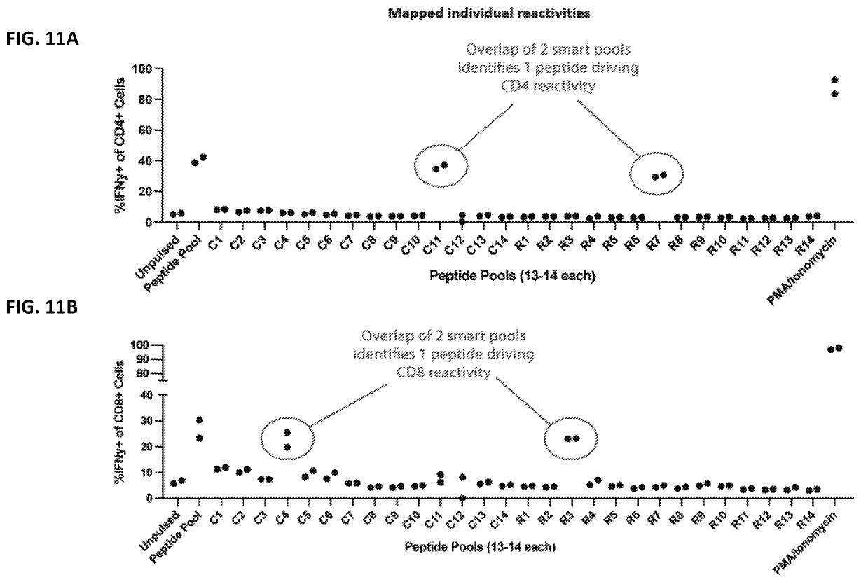

In order to assess selected TIL for function in response to non-specific polyclonal stimulation, bulk and selected TIL from CRC (n=3) and melanoma (n=1) were stimulated overnight with a soluble CD3/CD28 activator (e.g., for CD134+CD137+ expression), or were stimulated for 5 hours with PMA/Ionomycin (e.g., for IFNγ, TNFα and CD107a expression). Cells were then assessed for CD134+CD1374, IFNγ, TNFα and CD107a expression on CD4+ (FIG. 10A) and CD8+ (FIG. 10B) cells by flow cytometry. In response to non-specific, polyclonal stimulation (CD3/CD28 or PMA/Ionomycin), selected and bulk TIL showed similar levels of T cell activation, cytokine production, and degranulation. Taken together these data demonstrate that selected TIL are functional. Individual peptide reactivities were deconvoluted and identified for CD4+ and CD8+ restricted reactivity within the selected TIL composition. Selected TIL after REP were co-cultured for 5 hours at an effector: target ratio of 5:1 with APC, and IFN-γ production was quantified by flow cytometry within the CD4+ (FIG. 8A) and CD8+ (FIG. 8B) compartments. APC were either unloaded (DMSO), loaded with the entire pool of 190 peptides or loaded with smart peptide pools comprised of 13-14 peptides. Unloaded APC were used as the baseline and PMA/Ionomycin treated T cells as the positive control (maximal response). As shown in FIG. 11A and FIG. 11B, within each T cell compartment a single peptide produced IFN-γ to levels equivalent to that produced in the pool co-culture. The overlap of the reactive pools was used to identify the specific reactive peptide.

### Example 3: Expansion and Identification of Neoantigen Reactive Tamor Infiltrating Lymphocytes (TIL) from Metastatic Colorectal (CRC) and GI Cancers

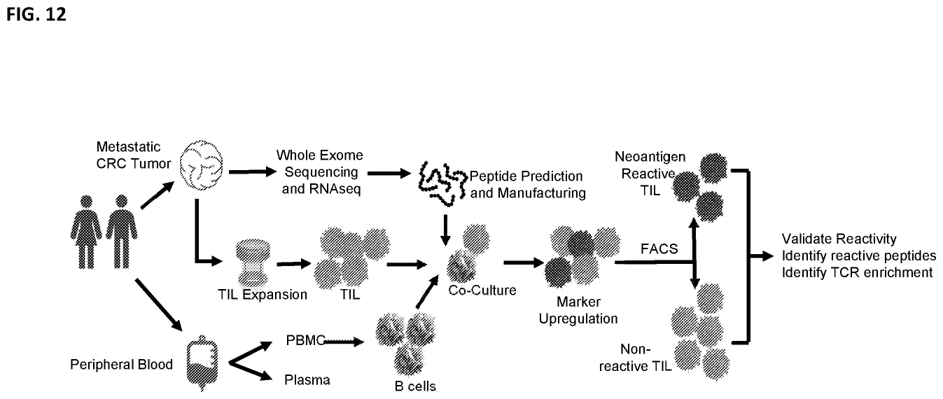

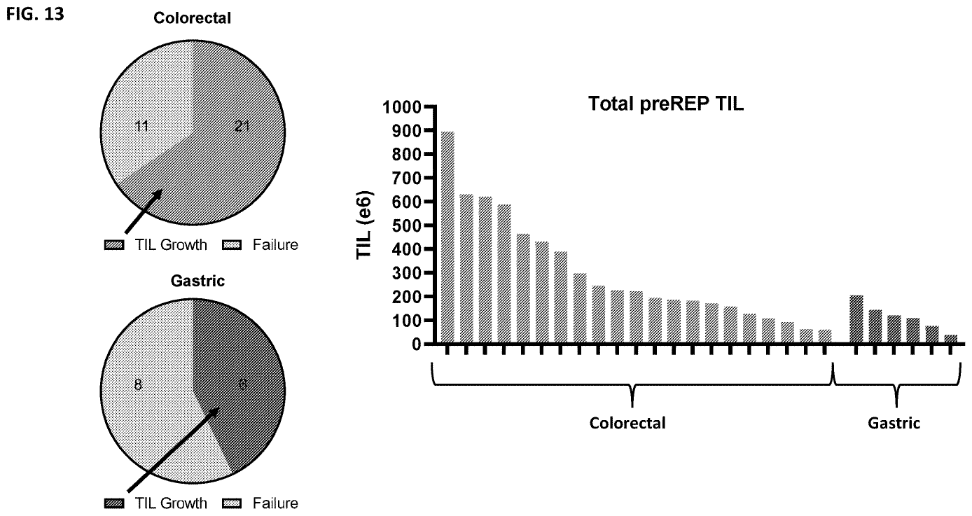

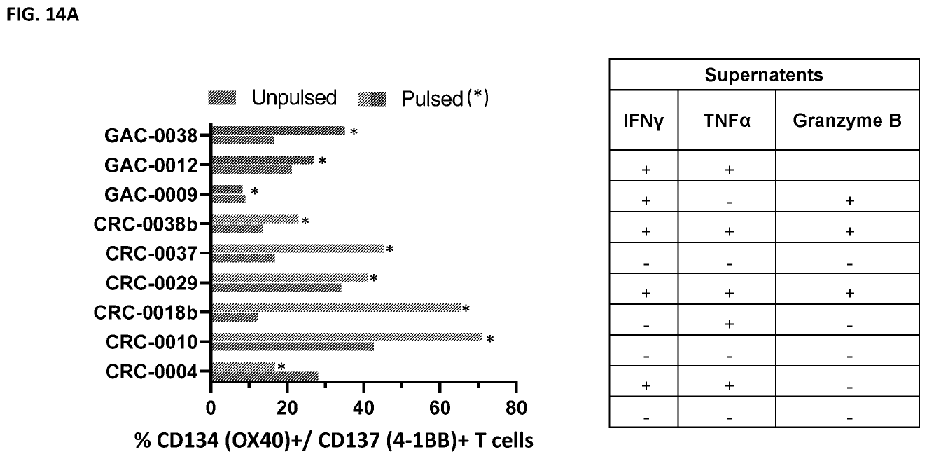

Substantially as described in Examples 1 and 2, tumor infiltrating lymphocyte (TIL) products with enriched reactivity to mutations expressed in neoantigens was generated from patient material. In a process depicted in FIG. 12, tumor infiltrating lymphocytes (TIL) were expanded from a digested colorectal cancer (CRC) tumor sample and antigen presenting cells (APCs) were generated from patient-matched peripheral blood. Mutations were predicted and peptides containing the potential neoantigens were manufactured. TIL from dissociated tumor cells of a patient were enriched for neoantigen reactivity by fluorescence single cell sorting (FACS) following coculture with autologous APCs pulsed with predicted peptides. The sorted neoantigen positive TIL (tumor reactive enriched-TIL composition) and TIL from the negative fraction (bystander cells) were expanded through REP, then validated for reactivity as well as for reactive peptides and TCR enrichment.

Colorectal and gastric cancer tumors were enzymatically and mechanically digested, and TIL was expanded in 6000 IU/mL IL-2. As shown in FIG. 13., 21 of 32 CRC samples and 6 of 14 gastric cancer samples expanded TIL to levels feasible for downstream enrichment. Total pre-REP TIL expanded following up to 4 weeks of culture ranged from 894×10⁶ to 38×10⁶. Therefore, TIL expansion was achieved in 64% of CRC samples and 43% of gastric cancer samples. Of those samples, pre-REP TIL from 6 CRC and 3 gastric cancers were sequenced, co-cultured, and sorted for neoantigen reactive TIL.

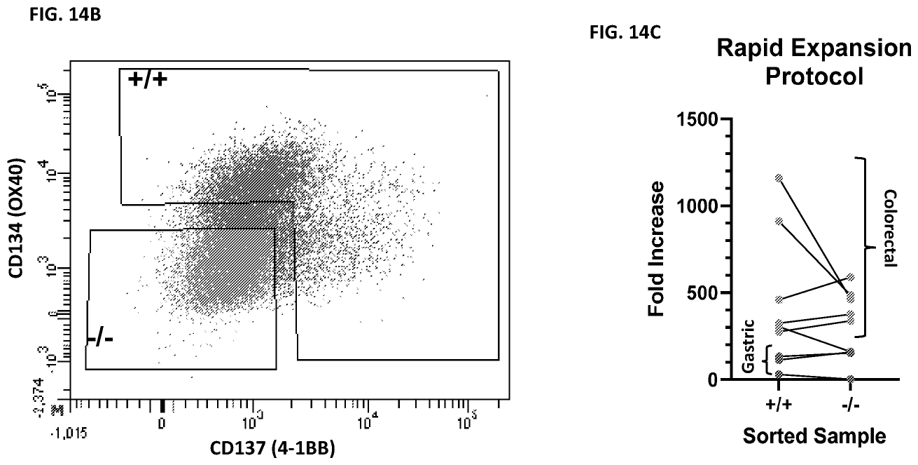

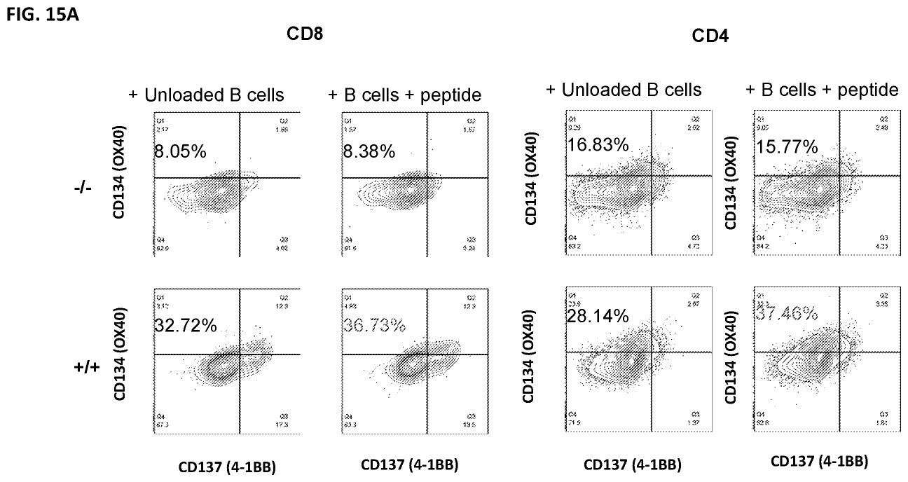

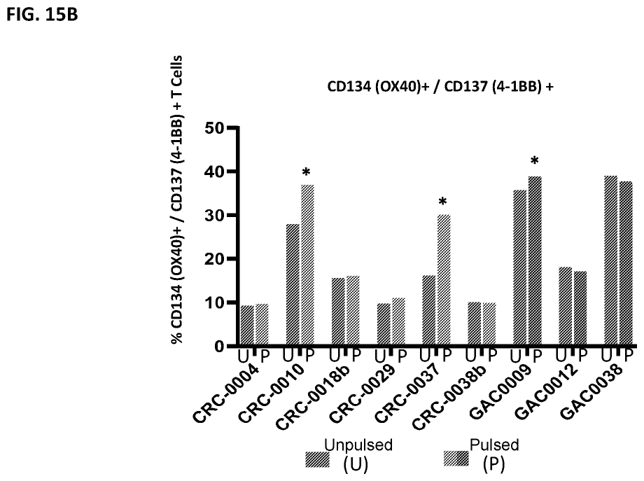

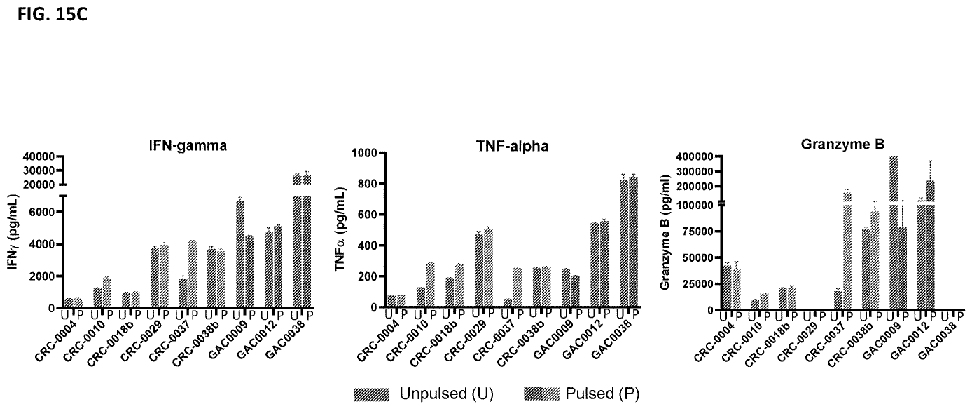

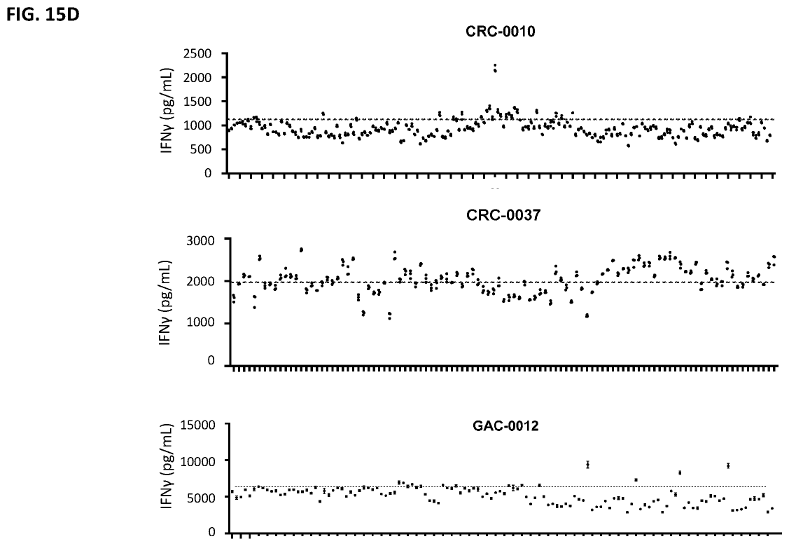

CRC (n=6) and gastric cancer (n=3) TIL were co-cultured with autologous B cells pulsed with neoantigen specific 25-mer peptides. TIL were sorted for CD137 (4-1BB)+and CD134 (OX40)+ upregulated TIL (+/+). As shown in FIG. 14A, upregulation of CD134 (OX40)/CD137 (4-1BB) was seen in 85% (5/6) of CRC and 66% (2/3) GI samples. Supernatants from sorting co-culture were tested for IFNγ, TNFα, and Granzyme B expression. A subset of these samples showed additional upregulation of Granzyme B, IFNγ, and or TNFα expression. An exemplary gating strategy for sorting of neoantigen peptide reactive (+/+) and non-reactive (−/−) TIL is shown in FIG. 14B. Neoantigen reactive (+/+) and non-reactive (−/−) TIL were expanded by rapid expansion protocol (REP) in FIG. 14C.

Following sorting of CD134 (OX40)/CD137 (4-1BB) positive TIL and REP, reactivity against pooled neoantigen peptide was validated in 3 of 6 CRC and 1 of 3 GI. Individual peptide screening identified multiple neoantigen peptides driving reactivity in these validated TIL samples. A representative sample of neoantigen reactive (+/+) and non-reactive (−/−) TIL following REP that were cocultured with autologous B cells pulsed with a pool of neoantigen specific 25-mer peptides to verify reactivity of sorted samples is shown in FIG. 15A. Reactivity was measured by CD137 (4-1BB) and CD134 (OX40) upregulation by flow analysis following co-culture against pooled peptides in FIG. 15B.

FIG. 15C depicts reactivity as measured by upregulation of Granzyme B, IFNγ, and TNFα secretion in supernatant in an ELLA assay. Peptides were further screened for individual reactivity by upregulation of IFNy secretion as shown in FIG. 15D.

Taken together, these data show TIL from metastatic colorectal cancer and gastric cancer patient samples expanded from multiple disease sites. Further, these data support that TIL from these samples can be screened for neoantigens and enriched for neoantigen-reactive TIL, and that these enriched TIL maintained increased reactivity against these predicted peptides upon restimulation when compared to TIL that did not upregulate CD134 (OX40)/CD137 (4-1BB). These data reinforce further investigation into the use of neoantigen-enriched TIL products to expand the utility of adoptive cell therapy.

### VIII. THE CONCLUSION

The foregoing descriptions of various embodiments of the invention have been presented for purposes of illustration and description. It is not intended to limit the invention to the precise forms disclosed. Many modifications, variations and refinements will be apparent to practitioners skilled in the art. For example, embodiments of the pharmaceutical T lymphocyte infiltrating (TIL) compositions described herein including those enriched in tumor reactive T cells can be adapted for treatment of a number of cancers including solid tumors such as GI, breast, bone and melanomas and various liquid/hematologic tumors such as leukemia, lymphoma, multiple myeloma and related diseases and related diseases. Also, those skilled in the art will recognize, or be able to ascertain using no more than routine experimentation, numerous equivalents to the specific devices and methods described herein. Such equivalents are considered to be within the scope of the present invention and are covered by the appended claims below.

Elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Further still, embodiments of the invention also contemplate the exclusion or negative recitation of an element, feature, chemical, therapeutic agent, characteristic, value or step wherever said element, feature, chemical, therapeutic agent, characteristic, value, step or the like is positively recited. Hence, the scope of the present invention is not limited to the specifics of the described embodiments, but is instead limited solely by the appended claims.

What is claimed:

1. A pharmaceutical T lymphocyte infiltrating (TIL) composition enriched in tumor reactive T cells, the pharmaceutical composition comprising a multiclonal population of T cells comprising CD4+ and CD8+ T cells from a tumor, wherein the population comprises at least 10 different T cell receptor (TCR) clonotypes each with a frequency in the population of at least 2.0%; and wherein at least 90% of the cells in the composition are CD3+ T cells.

2. The pharmaceutical composition of claim 1, wherein at least 11 different TCR clonotypes have a frequency in the population of at least 2.0%.

3. The pharmaceutical composition of claim 1, wherein at least 12 different TCR clonotypes have a frequency in the population of at least 2.0%.

4. A pharmaceutical T lymphocyte infiltrating (TIL) composition enriched in tumor reactive T cells, the pharmaceutical composition comprising a multiclonal population of T cells comprising CD4+ and CD8+ T cells from a tumor, wherein the population comprises at least 10 different T cell receptor (TCR) clonotypes each with a frequency in the population of at least 1.0%; and wherein at least 90% of the cells in the composition are CD3+ T cells.

5. The pharmaceutical composition of claim 4, wherein at least 11 different TCR clonotypes have a frequency in the population of at least 1.0%.

6. The pharmaceutical composition of claim 4, wherein at least 12 different TCR clonotypes have a frequency in the population of at least 1.0%.

7. The pharmaceutical composition of any of claims 1-6, wherein 8 to 15 different T cell receptor (TCR) clonotypes make up at least 50% of the TCR frequency in the population.

8. A pharmaceutical composition enriched in tumor reactive T cells, the pharmaceutical composition comprising a multiclonal population of tumor infiltrating lymphocytes comprising CD4+ and CD8+ T cells from a tumor, wherein 8 to 15 different T cell receptor (TCR) clonotypes make up at least 50% of the TCR frequency in the population.

9. The pharmaceutical composition of any of claims 1-8, wherein 9 to 12 different TCR clonotypes make up at least 50% of the TCR frequency in the population.

10. The pharmaceutical composition of any of claims 1-9, wherein the TCR clonotypes exhibit reactivity to at least one CD4 antigen and at least one CD8 antigen.

11. The pharmaceutical composition of any of claims 1-10, wherein the TCR clonotypes exhibit reactivity for 2 to 100 different peptide antigens.

12. The pharmaceutical composition of any of claims 1-11, wherein the TCR clonotypes exhibit reactivity for 10 to 40 different peptide antigens.

13. The pharmaceutical composition of any of claims 1-11, wherein the TCR clonotypes exhibit reactivity for 2 to 6 different peptide antigens.

14. The pharmaceutical composition of any of claims 1-11, wherein the TCR clonotypes exhibit reactivity for 2 to 4 peptide antigens.

15. The pharmaceutical composition of any of claims 1-11, wherein the TCR clonotypes exhibit reactivity for 2 peptide antigens.

16. The pharmaceutical composition of any of claims 1-11 and 13-15, wherein the TCR clonotypes exhibit reactivity for one CD8 antigen and one CD4 antigen.

17. A pharmaceutical composition enriched in tumor reactive T cells, the pharmaceutical composition comprising a multiclonal population of tumor infiltrating lymphocytes comprising CD4+ and CD8+ T cells from a tumor, wherein 10 to 100 different T cell receptor (TCR) clonotypes are present in the population.

18. The pharmaceutical composition of claim 17, wherein the TCR clonotypes exhibit reactivity for 10 to 40 different peptide antigens.

19. A pharmaceutical composition enriched in tumor reactive T cells, the pharmaceutical composition comprising a multiclonal population of tumor infiltrating lymphocytes comprising CD4+ and CD8+ T cells from a tumor, wherein 20 to 100 different T cell receptor (TCR) clonotypes are present in the population.

20. The pharmaceutical composition of claim 19, wherein 20 to 60 different TCR clonotypes are present in the population.

21. A pharmaceutical composition enriched in tumor reactive T cells, the pharmaceutical composition comprising a multiclonal population of tumor infiltrating lymphocytes comprising CD4+ and CD8+ T cells from a tumor, wherein the top 40 TCR clonotypes make up at least 75% of the TCR frequency in the population.

22. The pharmaceutical composition of any of claims 5-21, wherein at least 90% of the cells in the population are CD3+ T cells.

23. The pharmaceutical composition of any of claims 1-22, wherein the TCR clonotypes exhibit reactivity for at least one CD8 antigen and at least one CD4 antigen.

24. The pharmaceutical composition of any of claims 1-23, wherein at least 20% of the CD8+ T cells and/or at least 20% of the CD4+ T cells in the composition exhibit neoantigen reactivity.

25. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein at least 20% of the CD8+ T cells and/or at least 20% of the CD4+ T cells in the composition exhibit neoantigen reactivity.

26. The pharmaceutical composition of claim 24 or claim 25, wherein at least 25% of the CD8+ T cells and/or at least 20% of the CD4+ T cells in the composition exhibit neoantigen reactivity.

27. The pharmaceutical composition of claim 24 or claim 25, wherein at least 30% of the CD8+ T cells and/or at least 20% of the CD4+ T cells in the composition exhibit neoantigen reactivity.

28. The pharmaceutical composition of claim 24 or claim 25, wherein at least 40% of the CD8+ T cells and/or at least 30% of the CD4+ T cells in the composition exhibit neoantigen reactivity.

29. The pharmaceutical composition of any of claims 19-28, wherein neoantigen reactivity is determined in a co-culture assay with peptide loaded autologous APCs (e.g., as described in Example 2) by one or more of: upregulation of CD134 and CD137, IFN-γ production, TNF-alpha production, granzyme B production or degranulation, optionally wherein degranulation is determined based on CD107 expression.

30. The pharmaceutical composition of any of claims 1-29, wherein the TIL composition is characterized by at least a 1.5-fold increased percentage of cells positive for CD134 and CD137 compared to a bulk TIL population in a co-culture assay with peptide loaded autologous APCs, optionally at least a 2-fold, at least a 3-fold or at least a 4-fold increase in CD134 and CD137 positive cells compared to a bulk TIL population.

31. The pharmaceutical composition of any of claims 1-11, wherein greater than 30% of the cells in the TIL composition are positive for CD134 and CD137 in a co-culture assay with peptide loaded autologous APCs, optionally greater than about 35%, greater than about 40%, or greater than about 45% of cells are positive for CD134 and CD137.

32. The pharmaceutical composition of any of claims 1-31, wherein greater than 48% of the cells in the TIL composition are positive for CD134 and CD137 in a co-culture assay with peptide loaded autologous APCs, optionally greater than about 50%, greater than about 60%, or greater than about 70% of cells are positive for CD134 and CD137.

33. The pharmaceutical composition of any of claims 1-32, wherein, the TIL composition is characterized by at least one of the following criteria in an in vitro co-culture assay with peptide loaded autologous APC.
i) IFN-γ production that is greater than 1,000 pg/mL;
(ii) TNF-alpha production that is greater than 100 pg/mL;
(iii) greater than 10% CD107a+ cells; and
iv) granzyme B production that is greater than 10,000 pg/mL.

34. The pharmaceutical composition of any of claims 1-33, wherein, the TIL composition is characterized by at least one of the following criteria in an in vitro co-culture assay with peptide loaded autologous APC:
i) IFN-γ production that is greater than 100,000 pg/mL;
(ii) TNF-alpha production that is greater than 250 pg/mL;
(iii) greater than 10% CD107a+ cells; and
iv) granzyme B production that is greater than 50,000 pg/mL.

35. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following criteria in an in vitro co-culture assay with peptide loaded autologous APC:
i) IFN-γ production that is greater than 1,000 pg/mL;
(ii) TNF-alpha production that is greater than 100 pg/ml;
(iii) greater than 10% CD107a+ cells; and
(iv) granzyme B production that is greater than 10,000 pg/mL.

36. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following criteria in an in vitro co-culture assay with peptide loaded autologous APC:
i) IFN-γ production that is greater than 100,000 pg/mL;
(ii) TNF-alpha production that is greater than 250 pg/mL;
(iii) greater than 10% CD107a+ cells; and
(iv) granzyme B production that is greater than 50,000 pg/mL.

37. The pharmaceutical composition of claims 33-36, wherein the TIL composition is characterized by at least two of criteria (i)-(iv).

38. The pharmaceutical composition of claims 33-36, wherein the TIL composition is characterized by at least three of criteria (i)-(iv).

39. The pharmaceutical composition of claims 33-36, wherein the TIL composition is characterized by criteria (i)-(iv).

40. The pharmaceutical composition of any of claims 29-39, wherein the TIL composition is characterized by IFN-γ production that is greater than 2,500 pg/mL, greater than 5,000 pg/mL, greater than 10,000 pg/mL, greater than 25,000 pg/mL, greater than 50,000 pg/mL, greater than 100,000 pg/mL, greater than 200,000 pg/mL, greater than 250,000 pg/mL, greater than 500,000 pg/mL, or greater than 1,000,000 pg/mL.

41. The pharmaceutical composition of any of claims 29-40, wherein the TIL composition is characterized by IFN-γ production that is greater than 250,000 pg/mL, greater than 500,000 pg/mL, or greater than 1,000,000 pg/mL.

42. The pharmaceutical composition of any of claims 29-41, wherein the TIL composition is characterized by TNF-alpha production that is greater than 200 pg/mL., greater than 500 pg/mL, greater than 1000 pg/mL, or greater than 2000 pg/mL.

43. The pharmaceutical composition of any of claims 29-42, wherein the TIL composition is characterized by TNF-alpha production that is greater 500 pg/mL, greater than 1000 pg/mL, or greater than 2000 pg/mL.

44. The pharmaceutical composition of any of claims 29-43, wherein the TIL composition is characterized by greater than 15% CD107a+ cells, greater than 20% CD107a cells, or greater than 25% CD107a+ cells.

45. The pharmaceutical composition of any of claims 29-44, wherein the TIL composition is characterized by granzyme B production that is greater than 15,000 pg/mL, greater than 25,000 pg/mL, greater than 50,000 pg/mL, greater than 100,000 pg/mL, greater than 200,000 pg/mL, greater than 300,000 pg/mL, greater than 400,000 pg/mL or greater than 500,000 pg/mL.

46. The pharmaceutical composition of any of claims 29-45, wherein the TIL composition is characterized by granzyme B production that is greater than 200,000 pg/mL, greater than 300,000 pg/mL, greater than 400,000 pg/mL or greater than 500,000 pg/mL.

47. The pharmaceutical composition of any of claims 1-46, wherein, the TIL composition is characterized by at least one of the following criteria in an in vitro co-culture assay with peptide loaded autologous APC:
i) IFN-γ that is 50-fold or higher than a bulk TIL composition that is not enriched for tumor reactive T cells;
ii) TNF-α that is 300-fold or higher than a bulk TIL composition that is not enriched for tumor reactive T cells; or
iii) granzyme B that is 15-fold or higher than a bulk TIL composition that is not enriched for tumor reactive T cells.

48. A pharmaceutical T lymphocyte infiltrating (TIL) composition enriched in tumor reactive T cells, the pharmaceutical composition comprising tumor infiltrating lymphocytes comprising CD4+ and CD8+ T cells from a tumor, wherein at least 90% of cells in the composition are CD3+ T cells and wherein, the TIL composition is characterized by at least one of the following criteria in an in vitro co-culture assay with peptide loaded autologous APC:
i) IFN-γ that is 50-fold or higher than a bulk TIL composition that is not enriched for tumor reactive T cells;
ii) TNF-α that is 300-fold or higher than a bulk TIL composition that is not enriched for tumor reactive T cells; or
iii) granzyme B that is 15-fold or higher from a bulk TIL composition that is not enriched for tumor reactive T cells.

49. The pharmaceutical composition of claim 47 or claim 48, wherein the TIL composition is characterized by criteria (i) and (ii).

50. The pharmaceutical composition of claim 47 or claim 48, wherein the TIL composition is characterized by criteria (i) and (iii).

51. The pharmaceutical composition of claim 47 or claim 48, wherein the TIL composition is characterized by criteria (ii) and (iii).

52. The pharmaceutical composition of claim 47 or claim 48, wherein the TIL composition is characterized by criteria (i), (ii) and (iii).

53. The pharmaceutical composition of any of claims 1-52, wherein the composition is characterized by a greater number of CD4+ T cells than CD8+ T cells.

54. The pharmaceutical composition of any of claims 1-52, wherein a ratio of CD4+ T cells to CD8+ T cells in the composition is between 5:1 to 1:5.

55. The pharmaceutical composition of any of claims 1-52, wherein a ratio of CD4+ T cells to CD8+ T cells in the composition is between 5:1 to 50:1, between 5:1 to 25:1, between 5:1 to 20:1, between 5:1 to 15:1, between 5:1 to 10:1, between 10:1 to 50:1, between 10:1 to 25:1, between 10:1 to 20:1, between 10:1 to 15:1, between 15:1 to 50:1, between 15:1 to 25:1, between 15:1 to 20:1, between 20:1 to 50:1, between 20:1 to 25:1 or between 25:1 to 50:1.

56. The pharmaceutical composition of any of claims 1-55, wherein a ratio of CD4+ T cells to CD8+ T cells in the composition is at or about 10:1 to 25:1, optionally at or about 20:1.

57. The pharmaceutical composition of any of claims 1-56, wherein greater than 50% of the CD3+ T cells, optionally greater than 50% of the CD4 and CD8+ T cells, express markers of an effector memory phenotype.

58. The pharmaceutical composition of any of claims 1-57, wherein greater than 75% of the CD3+ T cells, optionally greater than 75% of the CD4 and CD8+ T cells, express markers of an effector memory phenotype.

59. The pharmaceutical composition of any of claims 1-58, wherein greater than 80% of the CD3+ T cells, optionally greater than 80% of the CD4 and CD8+ T cells, express markers of an effector memory phenotype.

60. The pharmaceutical composition of any of claims 1-58, wherein greater than 85% of the CD3+ T cells, optionally greater than 85% of the CD4 and CD8+ T cells, express markers of an effector memory phenotype.

61. The pharmaceutical composition of any of claims 1-58, wherein greater than 90% of the CD3+ T cells, optionally greater than 90% of the CD4 and CD8+ T cells, express markers of an effector memory phenotype.

62. The pharmaceutical composition of any of claims 58-61, wherein the effector memory phenotype is characterized by surface marker expression of one or more of CD45RA⁻, CD45RO+, CD62L⁻, CCR7−, CD28− and CD27−.

63. The pharmaceutical composition of any of claims 58-62, wherein the effector memory phenotype is characterized by surface marker expression CD45RA⁻, CD45RO+, CD62L⁻, and CCR7⁻.

64. The pharmaceutical composition of any of claims 58-62, wherein the effector memory phenotype is characterized by surface marker expression CD45RA⁻, CD45RO⁺, CD62L⁻, CCR7⁻, CD28⁻ and CD27⁻.

65. The pharmaceutical composition of any of claims 58-62, wherein the effector memory phenotype is characterized by surface marker expression CD45RA⁻ and CCR7⁻.

66. The pharmaceutical composition of any of claims 1-65, wherein greater than 95% of the CD4+ and CD8+ T cells in the composition are PD-1⁻.

67. The pharmaceutical composition of any of claims 1-66, wherein greater than 80% of the CD4+ and CD8+ T cells in the composition LAG3⁻.

68. The pharmaceutical composition of any of claims 1-67, wherein the number of cells in the composition, or of viable cells thereof, is at least 2×10⁷ cells.

69. The pharmaceutical composition of any of claims 1-68, wherein the number of cells in the composition, or of viable cells thereof, is between at or about 2×10⁷ cells and 20×10⁹ cells, 2×10⁷ cells and 10×10⁹ cells, 2×10⁷ cells and 2×10⁹ cells, 2×10⁷ cells and 2×10⁸ cells, 2×10⁸ cells and 20×10⁹ cells, 2×10⁸ cells and 10×10⁹ cells, 2×10⁸ cells and 2×10⁹ cells, 2×10⁹ cells and 20×10⁹ cells, 2×10⁹ cells and 10×10⁹ cells, or 10×10⁹ cells and 20×10⁹ cells, each inclusive.

70. The pharmaceutical composition of any of claims 1-69, wherein the pharmaceutical composition is for treatment of a patient's tumor.

71. The pharmaceutical composition of any of claims 1-70, wherein the tumor is a colorectal cancer (CRC) tumor, a melanoma tumor, a non-small cell lung cancer (NSCLC) tumor, or an ovarian cancer tumor.

72. The pharmaceutical composition of any of claims 1-71, wherein the tumor is from a human subject.

73. The pharmaceutical composition of claim 72, wherein the pharmaceutical composition is for autologous adoptive therapy to the human subject.

74. The pharmaceutical composition of any of claims 1-73, comprising a pharmaceutically acceptable excipient.

75. The pharmaceutical composition of any of claims 1-74, comprising a cryoprotectant.

76. The pharmaceutical composition of any of claims 1-75, wherein the composition is a liquid composition.

77. The pharmaceutical composition of claim 76, wherein the composition had been frozen and thawed.

78. The pharmaceutical composition of any of claims 1-77, wherein the volume of the composition is between 1 mL and 500 mL.

79. The pharmaceutical composition of any of claims 1-78, wherein the composition is frozen.

80. A pharmaceutical T lymphocyte infiltrating (TIL) composition enriched in tumor reactive T cells for treatment of a patient's tumor, the pharmaceutical composition comprising a multiclonal population of T cells comprising CD4+ and CD8+ T cells from the patient's tumor, wherein the population comprises at least 10 different T cell receptor (TCR) clonotypes each with a frequency in the population of at least 2.0%; and wherein at least 90% of the cells in the composition are CD3+ T cells.

81. A pharmaceutical T lymphocyte infiltrating (TIL) composition enriched in tumor reactive T cells for treatment of a patient's tumor, the pharmaceutical composition comprising a multiclonal population of T cells comprising CD4+ and CD8+ T cells from the patient's tumor, wherein the population comprises at least 10 different T cell receptor (TCR) clonotypes each with a frequency in the population of at least 1.0%; and wherein at least 90% of the cells in the composition are CD3+ T cells.

82. The pharmaceutical composition of any of claims 1-81, wherein the TIL composition is produced by an ex vivo method comprising expansion of tumor-reactive T cells from a donor subject that have been co-cultured with autologous antigen presenting cells and peptide neoantigens.

83. The pharmaceutical composition of any of claims 1-82, wherein the TIL composition is produced by a method comprising:
a. providing dissociated tumor cells from a tumor obtained from a donor subject, wherein the dissociated tumor cells are a first population of T cells that comprise CD4+ and CD8+ T cells;
b. culturing the first population of T cells with recombinant IL-2 added at a concentration between 3000 IU/mL and 6000 IU/mL, inclusive, for 14 to 28 days to produce a second population of T cells;
c. co-culturing the second population of T cells for 12 to 48 hours with autologous antigen presenting cells (APCs) with recombinant IL-2 added at a concentration of 100 IU/mL to 1000 IU/mL to produce a third population of T cells, wherein the APCs are loaded with a pool of peptide neoantigens from the tumor, wherein each peptide is 13-40 amino acids in length and is loaded at a concentration of 100 ng/ml per peptide, and wherein the ratio of the second population of T cells to APCs is 2:1 to 10:1;
d. selecting cells from the third population of T cells that are surface positive for CD134 and/or CD137 to produce a fourth population of T cells; and
e. expanding tumor infiltrating lymphocytes (TILs) by incubating the fourth population of T cells with irradiated human peripheral blood mononuclear cells (iPBMCs) at a ratio of 100 to 500 IPBMC to cells of the fourth population of T cells with recombinant IL-2 added at a concentration between 3000 IU/mL and 6000 IU/mL, inclusive, and 10 to 50 ng/mL anti-CD3 antibody (OKT3) for 12 to 16 days to produce a therapeutic composition of TILs enriched in tumor reactive cells.

84. A frozen composition comprising the pharmaceutical composition of any of claims 1-83 and a cryoprotectant.

85. A method of producing a T lymphocyte infiltrating (TIL) composition enriched in tumor reactive T cells, the method comprising:
a. providing dissociated tumor cells from a tumor obtained from a donor subject, wherein the dissociated tumor cells are a first population of T cells that comprise CD4+ and CD8+ T cells;
b. culturing the first population of T cells with recombinant IL-2 added at a concentration between about 3000 IU/mL and 6000 IU/mL, inclusive, for about 14 to 28 days to produce a second population of T cells;
c. co-culturing the second population of T cells for about 12 to 48 hours with autologous antigen presenting cells (APCs) with recombinant IL-2 added at a concentration of 100 IU/mL to 1000 IU/mL to produce a third population of T cells, wherein the APCs are loaded with a pool of peptide neoantigens from the tumor, wherein each peptide is 13-40 amino acids in length and is loaded at a concentration of 100 ng/ml per peptide, and wherein the ratio of the second population of T cells to APCs is about 2:1 to 10:1;
d. selecting cells from the third population of T cells that are surface positive for CD134 and/or CD137 to produce a fourth population of T cells; and
e. expanding tumor infiltrating lymphocytes (TILs) by incubating the fourth population of T cells with irradiated human peripheral blood mononuclear cells (iPBMCs) at a ratio of about 100 to 500 iPBMC to cells of the fourth population of T cells with recombinant IL-2 added at a concentration between about 3000 IU/mL and 6000 IU/mL, inclusive, and 10 to 50 ng/ml anti-CD3 antibody (OKT3) for 12 to 16 days to produce a therapeutic composition of TILs enriched in tumor reactive cells.

86. A method of treating a subject having a cancer, the method comprising administering to a subject having a tumor a therapeutic dose of the composition of any of claims 1-84.

87. The method of claim 86, wherein the therapeutically effective dose is between about 1×10⁹ and 10×10⁹ T cells.

88. The method of claim 86, wherein the therapeutically effective dose is from more than 1 million to less than 100 million T cells per kilogram of body weight.

89. The method of claim 86, wherein the therapeutically effective dose is from more than 1 million to less than 10 million T cells per kilogram of body weight.

90. The method of claim 86, wherein the therapeutically effective dose is from at or about 10 million to at or about 50 million T cells per kilogram of body weight.

91. The method of any of claims 86-90, wherein the cells of the therapeutic composition are autologous to the subject.
